SEQUENCE LISTING (121 sequences)

SEQ ID NO: 1 - protein, 37 aa, Oryctolagus cuniculus
GLRKRLRKFRNKIKEKLKKIGQKIQGLLPKLAPRTDY

SEQ ID NO: 2 - protein, 37 aa, synthetic construct
  REGION 1..37: note = Description of sequence: Mutation sequence
GLRPRLRKFRNKIKEKLKKIGQKIQGLLPKLAPRTDY

SEQ ID NO: 3 - protein, 37 aa, synthetic construct
  REGION 1..37: note = Description of sequence: Mutation sequence
GLRKRLRPFRNKIKEKLKKIGQKIQGLLPKLAPRTDY

SEQ ID NO: 4 - protein, 37 aa, synthetic construct
  REGION 1..37: note = Description of sequence: Mutation sequence
GLRKRLRKFRPKIKEKLKKIGQKIQGLLPKLAPRTDY

SEQ ID NO: 5 - protein, 37 aa, synthetic construct
  REGION 1..37: note = Description of sequence: Mutation sequence
GLRKRLRKFRNKIKPKLKKIGQKIQGLLPKLAPRTDY

SEQ ID NO: 6 - protein, 37 aa, synthetic construct
  REGION 1..37: note = Description of sequence: Mutation sequence
GLRKRLRKFRNKIKEKLKKIGQKIQGLLPKPAPRTDY

SEQ ID NO: 7 - protein, 37 aa, synthetic construct
  REGION 1..37: note = Description of sequence: Mutation sequence
GLRKRLRKFRNKIKEKLKKIGQKIQGLLPKLAPRPDY

SEQ ID NO: 8 - protein, 37 aa, synthetic construct
  REGION 1..37: note = Description of sequence: Mutation sequence
GLKKRLRKFRNKIKEKLKKIGQKIQGLLPKLAPRTDY

SEQ ID NO: 9 - protein, 37 aa, synthetic construct
  REGION 1..37: note = Description of sequence: Mutation sequence
GLRKKLRKFRNKIKEKLKKIGQKIQGLLPKLAPRTDY

SEQ ID NO: 10 - protein, 37 aa, synthetic construct
  REGION 1..37: note = Description of sequence: Mutation sequence
GLRKRLKKFRNKIKEKLKKIGQKIQGLLPKLAPRTDY

SEQ ID NO: 11 - protein, 37 aa, synthetic construct
  REGION 1..37: note = Description of sequence: Mutation sequence
GLRKRLRKFKNKIKEKLKKIGQKIQGLLPKLAPRTDY

SEQ ID NO: 12 - protein, 37 aa, synthetic construct
  REGION 1..37: note = Description of sequence: Mutation sequence
GLRKRLRKFRNKIKEKLKKIGQKIQGLLPKLAPKTDY

SEQ ID NO: 13 - protein, 37 aa, synthetic construct
  REGION 1..37: note = Description of sequence: Mutation sequence
GLRKRLRKIRNKIKEKLKKIGQKIQGLLPKLAPRTDY

SEQ ID NO: 14 - protein, 37 aa, synthetic construct
  REGION 1..37: note = Description of sequence: Mutation sequence
GIRKRLRKFRNKIKEKLKKIGQKIQGLLPKLAPRTDY

SEQ ID NO: 15 - protein, 37 aa, synthetic construct
  REGION 1..37: note = Description of sequence: Mutation sequence
GLRKRIRKFRNKIKEKLKKIGQKIQGLLPKLAPRTDY

SEQ ID NO: 16 - protein, 37 aa, synthetic construct
  REGION 1..37: note = Description of sequence: Mutation sequence
GLRKRLRKFRNKIKEKIKKIGQKIQGLLPKLAPRTDY

SEQ ID NO: 17 - protein, 37 aa, synthetic construct
  REGION 1..37: note = Description of sequence: Mutation sequence
GLRKRLRKFRNKIKEKLKKIGQKIQGLLPKIAPRTDY

SEQ ID NO: 18 - protein, 48 aa, synthetic construct
  REGION 1..48: note = Description of sequence: Mutation Sequence
CWTKSIPPKPCGLRKRLKKIKNKIKEKLKKIGQKIQGLLPKLAPRTDY

SEQ ID NO: 19 - protein, 48 aa, synthetic construct
  REGION 1..48: note = Description of sequence: Mutation Sequence
CWTKSIPPKPCGLRKILKKIKNKIKEKLKKIGQKIQGLLPKLAPRTDY

SEQ ID NO: 20 - protein, 48 aa, synthetic construct
  REGION 1..48: note = Description of sequence: Mutation Sequence
CWTKSIPPKPCGLRKKLKKIRNKIKEKLKKIGQKIQGLLPKLAPRTDY

SEQ ID NO: 21 - protein, 48 aa, synthetic construct
  REGION 1..48: note = Description of sequence: Mutation Sequence
CWTKSIPPKPCGLRKRLRKIKNKIKEKLKKIGQKIQGLLPKLAPKTDY

SEQ ID NO: 22 - protein, 48 aa, synthetic construct
  REGION 1..48: note = Description of sequence: Mutation Sequence
CWTKSIPPKPCGCRKPLRKIRNKIKEKLKKIGQKIQGLLPKLAPRTDY

SEQ ID NO: 23 - protein, 48 aa, synthetic construct
  REGION 1..48: note = Description of sequence: Mutation Sequence
CWTKSIPPKPCGCRKPCRKIRNKIKEKLKKIGQKIQGLLPKLAPRTDY

SEQ ID NO: 24 - protein, 37 aa, synthetic construct
  REGION 1..37: note = Description of sequence: Mutation Sequence
GLRKRLKKIKNKIKEKLKKIGQKIQGLLPKLAPRTDY

SEQ ID NO: 25 - protein, 37 aa, synthetic construct
  REGION 1..37: note = Description of sequence: Mutation Sequence
GLRKILKKIKNKIKEKLKKIGQKIQGLLPKLAPRTDY

SEQ ID NO: 26 - protein, 37 aa, synthetic construct
  REGION 1..37: note = Description of sequence: Mutation Sequence
GLRKKLKKIRNKIKEKLKKIGQKIQGLLPKLAPRTDY

SEQ ID NO: 27 - protein, 37 aa, synthetic construct
  REGION 1..37: note = Description of sequence: Mutation Sequence
GLRKRLRKIKNKIKEKLKKIGQKIQGLLPKLAPKTDY

SEQ ID NO: 28 - protein, 37 aa, synthetic construct
  REGION 1..37: note = Description of sequence: Mutation Sequence
GCRKPLRKIRNKIKEKLKKIGQKIQGLLPKLAPRTDY

SEQ ID NO: 29 - protein, 37 aa, synthetic construct
  REGION 1..37: note = Description of sequence: Mutation Sequence
GCRKPCRKIRNKIKEKLKKIGQKIQGLLPKLAPRTDY

SEQ ID NO: 30 - protein, 48 aa, synthetic construct
  REGION 1..48: note = Description of sequence: Mutation Sequence
CWTKSIPPKPCGLRKRLRKIRNKIKEKLKKIGQKIQGLLPKLAPRTDY

SEQ ID NO: 31 - protein, 37 aa, synthetic construct; identical to SEQ ID NO: 24
  REGION 1..37: note = Description of sequence: Mutation Sequence

SEQ ID NO: 32 - protein, 37 aa, synthetic construct; identical to SEQ ID NO: 25
  REGION 1..37: note = Description of sequence: Mutation Sequence

SEQ ID NO: 33 - protein, 37 aa, synthetic construct; identical to SEQ ID NO: 26
  REGION 1..37: note = Description of sequence: Mutation Sequence

SEQ ID NO: 34 - protein, 37 aa, synthetic construct; identical to SEQ ID NO: 27
  REGION 1..37: note = Description of sequence: Mutation Sequence

SEQ ID NO: 35 - protein, 37 aa, synthetic construct; identical to SEQ ID NO: 28
  REGION 1..37: note = Description of sequence: Mutation Sequence

SEQ ID NO: 36 - protein, 37 aa, synthetic construct; identical to SEQ ID NO: 29
  REGION 1..37: note = Description of sequence: Mutation Sequence

SEQ ID NO: 37 - protein, 37 aa, synthetic construct
  REGION 1..37: note = Description of sequence: Mutation Sequence
GLRKKLKKIKNKIKEKLKKIGQKIQGLLPKLAPRTDY

SEQ ID NO: 38 - protein, 37 aa, synthetic construct
  REGION 1..37: note = Description of sequence: Mutation Sequence
GLKKKLKKIKNKIKEKLKKIGQKIQGLLPKLAPRTDY

SEQ ID NO: 39 - protein, 37 aa, synthetic construct
  REGION 1..37: note = Description of sequence: Mutation Sequence
GLKKKLKKIKNKIKEKLKKIGQKIQGLLPKLAPKTDY

SEQ ID NO: 40 - protein, 37 aa, synthetic construct
  REGION 1..37: note = Description of sequence: Mutation Sequence
GLRKILRKIRNKIKEKLKKIGQKIQGLLPKLAPRTDY

SEQ ID NO: 41 - protein, 37 aa, synthetic construct
  REGION 1..37: note = Description of sequence: Mutation Sequence
GLKKILKKIKNKIKEKLKKIGQKIQGLLPKLAPRTDY

SEQ ID NO: 42 - protein, 37 aa, synthetic construct
  REGION 1..37: note = Description of sequence: Mutation Sequence
GLKKKLRKIRNKIKEKLKKIGQKIQGLLPKLAPRTDY

SEQ ID NO: 43 - protein, 43 aa, synthetic construct
  REGION 1..43: note = Description of sequence: Mutation Sequence
CGGGLRKRLRKIRNKIKEKLKKIGQKIQGLLPKLAPRTDYGGC

SEQ ID NO: 44 - protein, 46 aa, synthetic construct
  REGION 1..46: note = Description of sequence: Mutation Sequence
CGGSGLRKRLRKIRNKIKEKLKKIGQKIQGLLPKLAPRTDYGSGGC

SEQ ID NO: 45 - protein, 84 aa, synthetic construct
  REGION 1..84: note = Description of sequence: Mutation Sequence
CGGGLRKRLRKIRNKIKEKLKKIGQKIQGLLPKLAPRTDYGAGGGLRKRLRKFRNKIKEKLKKIGQKIQGLLPKLAPRTDYGGC

SEQ ID NO: 46 - protein, 48 aa, synthetic construct; identical to SEQ ID NO: 30
  REGION 1..48: note = Description of sequence: Mutation Sequence

SEQ ID NO: 47 - protein, 59 aa, synthetic construct
  REGION 1..59: note = Description of sequence: Mutation Sequence
CWTKSIPPKPCGLRKRLRKIRNKIKEKLKKIGQKIQGLLPKLAPRTDYCWTKSIPPKPC

SEQ ID NO: 48 - protein, 37 aa, synthetic construct
  REGION 1..37: note = Description of sequence: Mutation Sequence
GCRKPCRKPRNKIKEKLKKIGQKIQGLLPKLAPRTDY

SEQ ID NO: 49 - protein, 48 aa, synthetic construct
  REGION 1..48: note = Description of sequence: Mutation Sequence
GLRKRLRKIRNKIKEKLKKIGQKIQGLLPKLAPRTDYCWTKSIPPKPC

SEQ ID NO: 50 - protein, 28 aa, Bos taurus
GGLRSLGRKILRAWKKYGPIIVPIIRIG

SEQ ID NO: 51 - protein, 39 aa, synthetic construct
  REGION 1..39: note = Description of sequence: Mutation Sequence
CWTKSIPPKPCGGLRSLGRKILRAWKKYGPIIVPIIRIG

SEQ ID NO: 52 - protein, 29 aa, synthetic construct
  REGION 1..29: note = Description of sequence: Mutation Sequence
CWTKSIPPKPCGGLRSLGRKILRAWKKYG

SEQ ID NO: 53 - protein, 32 aa, synthetic construct
  REGION 1..32: note = Description of sequence: Mutation Sequence
CRKPGGLRSLGRKILRAWKKYGPIIVPIIRIG

SEQ ID NO: 54 - protein, 22 aa, synthetic construct
  REGION 1..22: note = Description of sequence: Mutation Sequence
CRKPGGLRSLGRKILRAWKKYG

SEQ ID NO: 55 - protein, 28 aa, synthetic construct
  REGION 1..28: note = Description of sequence: Mutation Sequence
GGLKSLGKKILRAWKKYGPIIVPIIRIG

SEQ ID NO: 56 - protein, 28 aa, synthetic construct
  REGION 1..28: note = Description of sequence: Mutation Sequence
GGLRSLGKKILKAWKKYGPIIVPIIRIG

SEQ ID NO: 57 - protein, 28 aa, synthetic construct
  REGION 1..28: note = Description of sequence: Mutation Sequence
GGLRSLGRKILKAWKKYGPIIVPIIKIG

SEQ ID NO: 58 - protein, 28 aa, synthetic construct
  REGION 1..28: note = Description of sequence: Mutation Sequence
GGLKSLGKKILKAWKKYGPIIVPIIKIG

SEQ ID NO: 59 - protein, 28 aa, synthetic construct
  REGION 1..28: note = Description of sequence: Mutation Sequence
GGLRSLGRKILRAIKKYGPIIVPIIRIG

SEQ ID NO: 60 - protein, 28 aa, synthetic construct
  REGION 1..28: note = Description of sequence: Mutation Sequence
GGLRSLGRKILRAWKKIGPIIVPIIRIG

SEQ ID NO: 61 - protein, 28 aa, synthetic construct
  REGION 1..28: note = Description of sequence: Mutation Sequence
GGLRSLGRKILRAIKKIGPIIVPIIRIG

SEQ ID NO: 62 - protein, 28 aa, Bos taurus
GGARSLGRKALRAAKKAGPAIVPIIRIG

SEQ ID NO: 63 - protein, 60 aa, synthetic construct
  REGION 1..60: note = Description of sequence: Mutation Sequence
RRIRPRPPRLPRPRPRPLPFPRPGPRPIPRPLPFPRPGPRPIPRPLPFPRPGPRPIPRPL

SEQ ID NO: 64 - protein, 71 aa, synthetic construct
  REGION 1..71: note = Description of sequence: Mutation Sequence
CWTKSIPPKPCRRIRPRPPRLPRPRPRPLPFPRPGPRPIPRPLPFPRPGPRPIPRPLPFPRPGPRPIPRPL

SEQ ID NO: 65 - protein, 64 aa, synthetic construct
  REGION 1..64: note = Description of sequence: Mutation Sequence
CRKPRRIRPRPPRLPRPRPRPLPFPRPGPRPIPRPLPFPRPGPRPIPRPLPFPRPGPRPIPRPL

SEQ ID NO: 66 - protein, 16 aa, Bos taurus
RRIRPRPPRLPRPRPR

SEQ ID NO: 67 - protein, 27 aa, synthetic construct
  REGION 1..27: note = Description of sequence: Mutation Sequence
CWTKSIPPKPCRRIRPRPPRLPRPRPR

SEQ ID NO: 68 - protein, 20 aa, synthetic construct
  REGION 1..20: note = Description of sequence: Mutation Sequence
CRKPRRIRPRPPRLPRPRPR

SEQ ID NO: 69 - protein, 64 aa, synthetic construct; identical to SEQ ID NO: 65
  REGION 1..64: note = Description of sequence: Mutation Sequence

SEQ ID NO: 70 - protein, 60 aa, synthetic construct
  REGION 1..60: note = Description of sequence: Mutation Sequence
RRIRPRPPRLPRPRPRPLPIPRPGPRPIPRPLPIPRPGPRPIPRPLPFPRPGPRPIPRPL

SEQ ID NO: 71 - protein, 60 aa, synthetic construct
  REGION 1..60: note = Description of sequence: Mutation Sequence
RRIRPRPPRLPRPRPRPLPFPRPGPRPIPRPLPIPRPGPRPIPRPLPIPRPGPRPIPRPL

SEQ ID NO: 72 - protein, 60 aa, synthetic construct
  REGION 1..60: note = Description of sequence: Mutation Sequence
RRIRPRPPRLPRPRPRPLPIPRPGPRPIPRPLPIPRPGPRPIPRPLPIPRPGPRPIPRPL

SEQ ID NO: 73 - protein, 60 aa, synthetic construct
  REGION 1..60: note = Description of sequence: Mutation Sequence
KKIRPRPPRLPRPRPRPLPFPRPGPRPIPRPLPFPRPGPRPIPRPLPFPRPGPRPIPRPL

SEQ ID NO: 74 - protein, 60 aa, synthetic construct
  REGION 1..60: note = Description of sequence: Mutation Sequence
RKIRPRPPKLPRPRPRPLPFPRPGPRPIPRPLPFPRPGPRPIPRPLPFPRPGPRPIPRPL

SEQ ID NO: 75 - protein, 60 aa, synthetic construct
  REGION 1..60: note = Description of sequence: Mutation Sequence
KKIRPRPPKLPRPRPRPLPFPRPGPRPIPRPLPFPRPGPRPIPRPLPFPRPGPRPIPRPL

SEQ ID NO: 76 - protein, 16 aa, synthetic construct
  REGION 1..16: note = Description of sequence: Mutation Sequence
KKIRPRPPKLPRPRPR

SEQ ID NO: 77 - protein, 38 aa, Canis lupus
  REGION 1..16: note = Sample is from dog (Canis lupus familiaris)
RLKELITTGGQKIGEKIRRIGQRIKDFFKNLQPREEKS

SEQ ID NO: 78 - protein, 21 aa, Canis lupus
  REGION 1..21: note = Sample is from dog (Canis lupus familiaris)
RLKELITTGGQKIGEKIRRIG

SEQ ID NO: 79 - protein, 49 aa, synthetic construct
  REGION 1..49: note = Description of sequence: Mutation Sequence
CWTKSIPPKPCRLKELITTGGQKIGEKIRRIGQRIKDFFKNLQPREEKS

SEQ ID NO: 80 - protein, 32 aa, synthetic construct
  REGION 1..32: note = Description of sequence: Mutation Sequence
CWTKSIPPKPCRLKELITTGGQKIGEKIRRIG

SEQ ID NO: 81 - protein, 42 aa, synthetic construct
  REGION 1..42: note = Description of sequence: Mutation Sequence
CRKPRLKELITTGGQKIGEKIRRIGQRIKDFFKNLQPREEKS

SEQ ID NO: 82 - protein, 25 aa, synthetic construct
  REGION 1..25: note = Description of sequence: Mutation Sequence
CRKPRLKELITTGGQKIGEKIRRIG

SEQ ID NO: 83 - protein, 38 aa, synthetic construct
  REGION 1..38: note = Description of sequence: Mutation Sequence
RLKELITTGGQKIGEKIRRIGQRIKDIIKNLQPREEKS

SEQ ID NO: 84 - protein, 38 aa, synthetic construct
  REGION 1..38: note = Description of sequence: Mutation Sequence
KLKELITTGGQKIGEKIKRIGQRIKDFFKNLQPREEKS

SEQ ID NO: 85 - protein, 38 aa, synthetic construct
  REGION 1..38: note = Description of sequence: Mutation Sequence
RLKELITTGGQKIGEKIKKIGQRIKDFFKNLQPREEKS

SEQ ID NO: 86 - protein, 38 aa, synthetic construct
  REGION 1..38: note = Description of sequence: Mutation Sequence
RLKELITTGGQKIGEKIRKIGQRIKDFFKNLQPKEEKS

SEQ ID NO: 87 - protein, 38 aa, synthetic construct
  REGION 1..38: note = Description of sequence: Mutation Sequence
RLKELITTGGQKIGEKIKKIGQRIKDFFKNLQPKEEKS

SEQ ID NO: 88 - protein, 21 aa, synthetic construct
  REGION 1..21: note = Description of sequence: Mutation Sequence
RLKELITTGGQKIGEKIKKIG

SEQ ID NO: 89 - protein, 32 aa, synthetic construct
  REGION 1..32: note = Description of sequence: Mutation Sequence
CWTKSIPPKPCRLKELITTGGQKIGEKIKKIG

SEQ ID NO: 90 - protein, 36 aa, Sus scrofa
GRFRRLRKKTRKRLKKIGKVLKWIPPIVGSIPLGCG

SEQ ID NO: 91 - protein, 47 aa, synthetic construct
  REGION 1..47: note = Description of sequence: Mutation Sequence
CWTKSIPPKPCGRFRRLRKKTRKRLKKIGKVLKWIPPIVGSIPLGCG

SEQ ID NO: 92 - protein, 40 aa, synthetic construct
  REGION 1..40: note = Description of sequence: Mutation Sequence
CRKPGRFRRLRKKTRKRLKKIGKVLKWIPPIVGSIPLGCG

SEQ ID NO: 93 - protein, 24 aa, Sus scrofa
GRFRRLRKKTRKRLKKIGKVLKWI

SEQ ID NO: 94 - protein, 35 aa, synthetic construct
  REGION 1..35: note = Description of sequence: Mutation Sequence
CWTKSIPPKPCGRFRRLRKKTRKRLKKIGKVLKWI

SEQ ID NO: 95 - protein, 28 aa, synthetic construct
  REGION 1..28: note = Description of sequence: Mutation Sequence
CRKPGRFRRLRKKTRKRLKKIGKVLKWI

SEQ ID NO: 96 - protein, 24 aa, synthetic construct
  REGION 1..24: note = Description of sequence: Mutation Sequence
GRFRRLRKKTRKRLKKIGKVLKLI

SEQ ID NO: 97 - protein, 24 aa, synthetic construct
  REGION 1..24: note = Description of sequence: Mutation Sequence
GRLRRLRKKTRKRLKKIGKVLKLI

SEQ ID NO: 98 - protein, 24 aa, synthetic construct
  REGION 1..24: note = Description of sequence: Mutation Sequence
GVFRVLRKVTRVVLKVIGKVLKLI

SEQ ID NO: 99 - protein, 24 aa, synthetic construct
  REGION 1..24: note = Description of sequence: Mutation Sequence
GVLRVLRKVTRVVLKVIGKVLKLI

SEQ ID NO: 100 - protein, 21 aa, Sus scrofa
RRLRKKTRKRLKKIGKVLKWI

SEQ ID NO: 101 - protein, 32 aa, synthetic construct
  REGION 1..32: note = Description of sequence: Mutation Sequence
CWTKSIPPKPCRRLRKKTRKRLKKIGKVLKWI

SEQ ID NO: 102 - protein, 32 aa, synthetic construct
  REGION 1..32: note = Description of sequence: Mutation Sequence
CWTKSIPPKPCRKLRKKTRKRLKKIGKVLKLI

SEQ ID NO: 103 - protein, 59 aa, synthetic construct
  REGION 1..59: note = Description of sequence: Mutation Sequence
  VARIANT 1: note = This position may be absent, when present X is cysteine; when present positions 2-11 are also present
  VARIANT 2: note = This position may be absent, this position is only present when location 1 is also present, when present X is tryptophan; when present positions 3-11 are also present
  VARIANT 3: note = This position may be absent, this position is only present when locations 1-2 are also present, when present X is tyrosine; when present positions 4-11 are also present
  VARIANT 4: note = This position may be absent, this position is only present when location 1-3 are also present, when present X is lysine; when present positions 5-11 are also present
  VARIANT 5: note = This position may be absent, this position is only present when locations 1-4 are also present, when present X is serine; when present positions 6-11 are also present
  VARIANT 6: note = This position may be absent, this position is only present when locations 1-5 are also present, when present X is isoleucine; when present positions 7-11 are also present
  VARIANT 7: note = This position may be absent, this position is only present when locations 1-6 are also present, when present X is proline; when present positions 8-11 are also present
  VARIANT 8: note = This position may be absent, when absent positions 1-7 and 9-11 are also absent when locations 1-7 are also present, X is proline, when present in the absence of locations 1-7, this position is cytosine
  VARIANT 9: note = This position may be absent, when absent positions 1-8 and 10-11 are also absent, when locations 1-7 are also present, X is lysine, when present in the absence of locations 1-7, this position is cytosine is arginine
  VARIANT 10: note = This position may be absent, when absent positions 1-9 and 11 are also absent, when locations 1-7 are also present, X is proline, when present in the absence of locations 1-7, this position is lysine
  VARIANT 11: note = This position may be absent, when absent positions 1-10 are also absent, when locations 1-7 are also present, X is cytosine, when present in the absence of locations 1-7, this position is proline
  VARIANT 13: note = X can be C or L
  VARIANT 14: note = X can be R or K
  VARIANT 16: note = X can be any of P, A, V, I, L, M, F, Y, or W
  VARIANT 17: note = X can be C or L
  VARIANT 18: note = X can be R or K
  VARIANT 20: note = X can be I or K
  VARIANT 21: note = X can be R, I or K
  VARIANT 45: note = X can be R or K
  VARIANT 49: note = This position may be absent, when present X is cysteine; when present positions 50-60 are also present
  VARIANT 50: note = This position may be absent, this position is only present when location 49 is also present, when present X is tryptophan; when present positions 52-60 are also present
  VARIANT 51: note = This position may be absent, this position is only present when locations 49-50 are also present, when present X is tyrosine; when present positions 52-60 are also present
  VARIANT 52: note = This position may be absent, this position is only present when location 49-51 are also present, when present X is lysine; when present positions 53-60 are also present
  VARIANT 53: note = This position may be absent, this position is only present when locations 49-52 are also present, when present X is serine; when present positions 54-60 are also present
  VARIANT 54: note = This position may be absent, this position is only present when locations 49-53 are also present, when present X is isoleucine; when present positions 55-60 are also present
  VARIANT 55: note = This position may be absent, this position is only present when locations 49-54 are also present, when present X is proline; when present positions 56-60 are also present
  VARIANT 56: note = This position may be absent, this position is only present when locations 49-55 are also present, when present X is proline; when present positions 57-60 are also present
  VARIANT 57: note = This position may be absent, this position is only present when locations 49-56 are also present, when present X is lysine; when present positions 58-60 are also present
  VARIANT 58: note = This position may be absent, this position is only present when locations 49-58 are also present, when present X is proline; when present postion 60 is also present
  VARIANT 59: note = This position may be absent, this position is only present when locations 49-59 are also present, when present X is cytosine
XXXXXXXXXXXGXXKXXXKXXNKIKEKLKKIGQKIQGLLPKLAPXTDYXXXXXXXXXXX

SEQ ID NO: 104 - protein, 12 aa, synthetic construct
  REGION 1..12: note = Description of sequence: Branch sequence
CWTKSIPPKPCG

SEQ ID NO: 105 - protein, 11 aa, synthetic construct
  REGION 1..11: note = Description of sequence: Branch sequence
CWTKSIPPKPC

SEQ ID NO: 106 - protein, 37 aa, synthetic construct
  REGION 1..37: note = Description of sequence: Mutation Sequence
  VARIANT 2: note = X can be C or L
  VARIANT 5: note = X can be R, K, I, or P
  VARIANT 6: note = X can be L or C
  VARIANT 7: note = X can be L or R
  VARIANT 10: note = X can be R or K
  VARIANT 34: note = X can be R or K
GXRKXXXKIXNKIKEKLKKIGQKIQGLLPKLAPXTDY

SEQ ID NO: 107 - protein, 28 aa, synthetic construct
  REGION 1..28: note = Description of sequence: Mutation Sequence
  VARIANT 1: note = X can be G, CWTKSIPPKPC-G or CRKP-G
  VARIANT 3: note = X can be L or A
  VARIANT 4: note = X can be R or K
  VARIANT 8: note = X can be R or K
  VARIANT 10: note = X can be I or A
  VARIANT 12: note = X can be R or K
  VARIANT 14: note = X can be W, I or A
  VARIANT 17: note = misc_feature - - Xaa can be Y, I or A
  VARIANT 20: note = X can be I or A
  VARIANT 26: note = X can be R or K
XGXXSLGXKXLXAXKKXGPXIVPIIXIG

SEQ ID NO: 108 - protein, 5 aa, synthetic construct
  REGION 1..5: note = Description of sequence: Branch sequence
CRKPG

SEQ ID NO: 109 - protein, 29 aa, synthetic construct
  REGION 1..29: note = Description of sequence: Mutation Sequence
  VARIANT 1: note = This position may be absent, when present X is cysteine; when present positions 2-11 are also present
  VARIANT 2: note = This position may be absent, this position is only present when location 1 is also present, when present X is tryptophan; when present positions 3-11 are also present
  VARIANT 3: note = This position may be absent, this position is only present when locations 1-2 are also present, when present X is tyrosine; when present positions 4-11 are also present
  VARIANT 4: note = This position may be absent, this position is only present when location 1-3 are also present, when present X is lysine; when present positions 5-11 are also present
  VARIANT 5: note = This position may be absent, this position is only present when locations 1-4 are also present, when present X is serine; when present positions 6-11 are also present
  VARIANT 6: note = This position may be absent, this position is only present when locations 1-5 are also present, when present X is isoleucine; when present positions 7-11 are also present
  VARIANT 7: note = This position may be absent, this position is only present when locations 1-6 are also present, when present X is proline; when present positions 8-11 are also present
  VARIANT 8: note = This position may be absent, when absent positions 1-7 and 9-11 are also absent when locations 1-7 are also present, X is proline, when present in the absence of locations 1-7, this position is cytosine
  VARIANT 9: note = This position may be absent, when absent positions 1-8 and 10-11 are also absent, when locations 1-7 are also present, X is lysine, when present in the absence of locations 1-7, this position is cytosine is arginine
  VARIANT 10: note = This position may be absent, when absent positions 1-9 and 11 are also absent, when locations 1-7 are also present, X is proline, when present in the absence of locations 1-7, this position is lysine
  VARIANT 11: note = This position may be absent, when absent positions 1-10 are also absent, when locations 1-7 are also present, X is cytosine, when present in the absence of locations 1-7, this position is proline
XXXXXXXXXXXGGLRSLGRKILRAWKKYG

SEQ ID NO: 110 - protein, 71 aa, synthetic construct
  REGION 1..71: note = Description of sequence: Mutation Sequence
  VARIANT 1: note = This position may be absent, when present X is cysteine; when present positions 2-11 are also present
  VARIANT 2: note = This position may be absent, this position is only present when location 1 is also present, when present X is tryptophan; when present positions 3-11 are also present
  VARIANT 3: note = This position may be absent, this position is only present when locations 1-2 are also present, when present X is tyrosine; when present positions 4-11 are also present
  VARIANT 4: note = This position may be absent, this position is only present when location 1-3 are also present, when present X is lysine; when present positions 5-11 are also present
  VARIANT 5: note = This position may be absent, this position is only present when locations 1-4 are also present, when present X is serine; when present positions 6-11 are also present
  VARIANT 6: note = This position may be absent, this position is only present when locations 1-5 are also present, when present X is isoleucine; when present positions 7-11 are also present
  VARIANT 7: note = This position may be absent, this position is only present when locations 1-6 are also present, when present X is proline; when present positions 8-11 are also present
  VARIANT 8: note = This position may be absent, when absent positions 1-7 and 9-11 are also absent when locations 1-7 are also present, X is proline, when present in the absence of locations 1-7, this position is cytosine
  VARIANT 9: note = This position may be absent, when absent positions 1-8 and 10-11 are also absent, when locations 1-7 are also present, X is lysine, when present in the absence of locations 1-7, this position is cytosine is arginine
  VARIANT 10: note = This position may be absent, when absent positions 1-9 and 11 are also absent, when locations 1-7 are also present, X is proline, when present in the absence of locations 1-7, this position is lysine
  VARIANT 11: note = This position may be absent, when absent positions 1-10 are also absent, when locations 1-7 are also present, X is cytosine, when present in the absence of locations 1-7, this position is proline
  VARIANT 13: note = X can be R or K
  VARIANT 20: note = X can be R or K
  VARIANT 31: note = X can be F or I
  VARIANT 45: note = X can be F or I
  VARIANT 59: note = X can be F or I
XXXXXXXXXXXXXIRPRPPXLPRPRPRPLPXPRPGPRPIPRPLPXPRPGPRPIPRPLPXPRPGPRPIPRPL

SEQ ID NO: 111 - protein, 12 aa, synthetic construct
  REGION 1..12: note = Description of sequence: Branch sequence
CWTKSIPPKPCR

SEQ ID NO: 112 - protein, 5 aa, synthetic construct
  REGION 1..5: note = Description of sequence: Branch sequence
CRKPR

SEQ ID NO: 113 - protein, 16 aa, synthetic construct
  REGION 1..16: note = Description of sequence: Mutation Sequence
  VARIANT 1: note = X can be R or K
  VARIANT 9: note = X can be R or K
XXIRPRPPXLPRPRPR

SEQ ID NO: 114 - protein, 49 aa, synthetic construct
  REGION 1..49: note = Description of sequence: Mutation Sequence
  VARIANT 1: note = This position may be absent, when present X is cysteine; when present positions 2-11 are also present
  VARIANT 2: note = This position may be absent, this position is only present when location 1 is also present, when present X is tryptophan; when present positions 3-11 are also present
  VARIANT 3: note = This position may be absent, this position is only present when locations 1-2 are also present, when present X is tyrosine; when present positions 4-11 are also present
  VARIANT 4: note = This position may be absent, this position is only present when location 1-3 are also present, when present X is lysine; when present positions 5-11 are also present
  VARIANT 5: note = This position may be absent, this position is only present when locations 1-4 are also present, when present X is serine; when present positions 6-11 are also present
  VARIANT 6: note = This position may be absent, this position is only present when locations 1-5 are also present, when present X is isoleucine; when present positions 7-11 are also present
  VARIANT 7: note = This position may be absent, this position is only present when locations 1-6 are also present, when present X is proline; when present positions 8-11 are also present
  VARIANT 8: note = This position may be absent, when absent positions 1-7 and 9-11 are also absent when locations 1-7 are also present, X is proline, when present in the absence of locations 1-7, this position is cytosine
  VARIANT 9: note = This position may be absent, when absent positions 1-8 and 10-11 are also absent, when locations 1-7 are also present, X is lysine, when present in the absence of locations 1-7, this position is cytosine is arginine
  VARIANT 10: note = This position may be absent, when absent positions 1-9 and 11 are also absent, when locations 1-7 are also present, X is proline, when present in the absence of locations 1-7, this position is lysine
  VARIANT 11: note = This position may be absent, when absent positions 1-10 are also absent, when locations 1-7 are also present, X is cytosine, when present in the absence of locations 1-7, this position is proline
  VARIANT 29: note = X can be R or K
  VARIANT 30: note = X can be R or K
  VARIANT 38: note = X can be F or I
  VARIANT 39: note = X can be F or I
  VARIANT 45: note = X can be R or K
XXXXXXXXXXXGLKELITTGGQKIGEKIXXIGQRIKDXXKNLQPXEEKS

SEQ ID NO: 115 - protein, 32 aa, synthetic construct
  REGION 1..32: note = Description of sequence: Mutation Sequence
  VARIANT 1: note = This position may be absent, when present X is cysteine; when present positions 2-11 are also present
  VARIANT 2: note = This position may be absent, this position is only present when location 1 is also present, when present X is tryptophan; when present positions 3-11 are also present
  VARIANT 3: note = This position may be absent, this position is only present when locations 1-2 are also present, when present X is tyrosine; when present positions 4-11 are also present
  VARIANT 4: note = This position may be absent, this position is only present when location 1-3 are also present, when present X is lysine; when present positions 5-11 are also present
  VARIANT 5: note = This position may be absent, this position is only present when locations 1-4 are also present, when present X is serine; when present positions 6-11 are also present
  VARIANT 6: note = This position may be absent, this position is only present when locations 1-5 are also present, when present X is isoleucine; when present positions 7-11 are also present
  VARIANT 7: note = This position may be absent, this position is only present when locations 1-6 are also present, when present X is proline; when present positions 8-11 are also present
  VARIANT 8: note = This position may be absent, when absent positions 1-7 and 9-11 are also absent when locations 1-7 are also present, X is proline, when present in the absence of locations 1-7, this position is cytosine
  VARIANT 9: note = This position may be absent, when absent positions 1-8 and 10-11 are also absent, when locations 1-7 are also present, X is lysine, when present in the absence of locations 1-7, this position is cytosine is arginine
  VARIANT 10: note = This position may be absent, when absent positions 1-9 and 11 are also absent, when locations 1-7 are also present, X is proline, when present in the absence of locations 1-7, this position is lysine
  VARIANT 11: note = This position may be absent, when absent positions 1-10 are also absent, when locations 1-7 are also present, X is cytosine, when present in the absence of locations 1-7, this position is proline
  VARIANT 29: note = X can be R or K
  VARIANT 30: note = X can be R or K
XXXXXXXXXXXGLKELITTGGQKIGEKIXXIG

SEQ ID NO: 116 - protein, 35 aa, synthetic construct
  REGION 1..35: note = Description of sequence: Mutation Sequence
  VARIANT 1: note = This position may be absent, when present X is cysteine; when present positions 2-11 are also present
  VARIANT 2: note = This position may be absent, this position is only present when location 1 is also present, when present X is tryptophan; when present positions 3-11 are also present
  VARIANT 3: note = This position may be absent, this position is only present when locations 1-2 are also present, when present X is tyrosine; when present positions 4-11 are also present
  VARIANT 4: note = This position may be absent, this position is only present when location 1-3 are also present, when present X is lysine; when present positions 5-11 are also present
  VARIANT 5: note = This position may be absent, this position is only present when locations 1-4 are also present, when present X is serine; when present positions 6-11 are also present
  VARIANT 6: note = This position may be absent, this position is only present when locations 1-5 are also present, when present X is isoleucine; when present positions 7-11 are also present
  VARIANT 7: note = This position may be absent, this position is only present when locations 1-6 are also present, when present X is proline; when present positions 8-11 are also present
  VARIANT 8: note = This position may be absent, when absent positions 1-7 and 9-11 are also absent when locations 1-7 are also present, X is proline, when present in the absence of locations 1-7, this position is cytosine
  VARIANT 9: note = This position may be absent, when absent positions 1-8 and 10-11 are also absent, when locations 1-7 are also present, X is lysine, when present in the absence of locations 1-7, this position is cytosine is arginine
  VARIANT 10: note = This position may be absent, when absent positions 1-9 and 11 are also absent, when locations 1-7 are also present, X is proline, when present in the absence of locations 1-7, this position is lysine
  VARIANT 11: note = This position may be absent, when absent positions 1-10 are also absent, when locations 1-7 are also present, X is cytosine, when present in the absence of locations 1-7, this position is proline
  VARIANT 13: note = X can be R or V
  VARIANT 14: note = X can be F or L
  VARIANT 16: note = X can be R or V
  VARIANT 20: note = X can be K or V
  VARIANT 23: note = X can be K or V
  VARIANT 24: note = X can be R or V
  VARIANT 27: note = X can be K or V
  VARIANT 34: note = X can be W or L
XXXXXXXXXXXGXXRXLRKXTRXXLKXIGKVLKXI

SEQ ID NO: 117 - protein, 32 aa, synthetic construct
  REGION 1..32: note = Description of sequence: Mutation Sequence
  VARIANT 1: note = This position may be absent, when present X is cysteine; when present positions 2-11 are also present
  VARIANT 2: note = This position may be absent, this position is only present when location 1 is also present, when present X is tryptophan; when present positions 3-11 are also present
  VARIANT 3: note = This position may be absent, this position is only present when locations 1-2 are also present, when present X is tyrosine; when present positions 4-11 are also present
  VARIANT 4: note = This position may be absent, this position is only present when location 1-3 are also present, when present X is lysine; when present positions 5-11 are also present
  VARIANT 5: note = This position may be absent, this position is only present when locations 1-4 are also present, when present X is serine; when present positions 6-11 are also present
  VARIANT 6: note = This position may be absent, this position is only present when locations 1-5 are also present, when present X is isoleucine; when present positions 7-11 are also present
  VARIANT 7: note = This position may be absent, this position is only present when locations 1-6 are also present, when present X is proline; when present positions 8-11 are also present
  VARIANT 8: note = This position may be absent, this position is only present when locations 1-7 are also present, when present X is proline; when present positions 9-11 are also present
  VARIANT 9: note = This position may be absent, this position is only present when locations 1-8 are also present, when present X is lysine; when present positions 10-11 are also present
  VARIANT 10: note = This position may be absent, this position is only present when locations 1-9 are also present, when present X is proline; when present positions 11-11 are also present
  VARIANT 11: note = This position may be absent, this position is only present when locations 1-10 are also present, when present X is cytosine
  VARIANT 13: note = X can be R or K
  VARIANT 31: note = X can be W or L
XXXXXXXXXXXRXLRKKTRKRLKKIGKVLKXI

SEQ ID NO: 118 - protein, 37 aa, Homo sapiens
LLGDFFRKSKEKIGKEFKRIVQRIKDFLRNLVPRTES

SEQ ID NO: 119 - protein, 23 aa, synthetic construct
  REGION 1..23: note = Description of sequence: Mutation Sequence
RIIDLLWRVRRPQKPKFVTVWVR

SEQ ID NO: 120 - protein, 27 aa, synthetic construct
  REGION 1..27: note = Description of sequence: Mutation Sequence
KILRGVCKKIMRTFLRRISKDILTGKK

SEQ ID NO: 121 - protein, 57 aa, synthetic construct
  REGION 1..57: note = Description of sequence: Mutation Sequence
MSTKDFNLDLVSVSKKDSGASPRITSISLCTPGCKTGALMGCNMKTATCHCSIHVSK

(19) United States
(12) Patent Application Publication
(10) Pub. No.: US 2025/0034218 A1
(43) Pub. Date: Jan. 30, 2025

(54) ANTIMICROBIAL PEPTIDES

(71) Applicant: BiomEdit, LLC, Fishers, IN (US)

(72) Inventors: Dwi Susanti, Fishers, IN (US); Arvind Kumar, Fishers, IN (US); Dharanesh Mahimapura Gangaiah, Fishers, IN (US)

(21) Appl. No.: 18/693,600

(22) PCT Filed: Sep. 21, 2022
(86) PCT No.: PCT/US2022/044221

§ 371 Date: Mar. 20, 2024

Related U.S. Application Data
(60) Provisional application No. 63/246,615, filed on Sep. 21, 2021.
(60) Provisional application No. 63/248,416, filed on Sep. 24, 2021.

(51) Int. Cl. C07K 14/47; A61K 38/00; A61P 31/04

(52) U.S. Cl. CPC C07K 14/4723; A61P 31/04; A61K 38/00

(57) ABSTRACT

Provided are engineered polypeptides including CAP18 variants, and other engineered cathelicidin polypeptides based on BMAP28, BAC7, K9CATH and PMAP36. Also provided are methods for inhibiting growth of at least one methanogen in an animal. Further provided are methods of reducing greenhouse gas emissions, such as methane emissions, that use such compositions. The compositions and methods include one or more of the antimicrobial peptides including CAP 18 variants and other engineered cathelicidin polypeptides based on BMAP28, BAC7, K9CATH and PMAP36.

### CROSS REFERENCE TO RELATED APPLICATIONS

The present application is a national stage application which claims priority from PCT Application No. PCT/US2022/044221 filed Sep. 21, 2022, which in turn claims priority to U.S. Applications Nos. 63/246,615 filed Sep. 21, 2021 and 63/248,416, filed Sep. 24, 2021, the entire contents of which are incorporated by reference herein.

### SEQUENCE LISTING XML

The instant application contains a Sequence Listing, conforming to the rules of WIPO Standard ST.26, submitted in XML format which was filed electronically by EFS-web via PatentCenter and is hereby incorporated by reference in its entirety. Said XML format Sequence Listing, created on Oct. 2, 2024, is named “2950-4 PCT/US_ST26.xml” and is 218,981 bytes in size.

### FIELD

The present disclosure relates to antimicrobial peptides and their use and application for reduction in bacterial colonization and for prevention and treatment of bacterial infection and disease in animals. The present disclosure also relates to antimicrobial peptides and their use and application for reduction of greenhouse gas emissions, particularly, methane emissions from animals such as livestock and, more particularly, ruminants.

### BACKGROUND

The widespread use of antibiotics has contributed to the selection for microorganisms with antibiotic resistance and to the selection for transmission of antibiotic resistance mechanisms between quite distantly related organisms. The number of resistant superbugs is increasing and new anti-infective solutions are urgently needed, but the time for development of a new antimicrobial drug is lengthy compared to the current dissemination of novel antibiotic resistance mechanisms among commensal and pathogenic microorganisms (French GL. (2010) Int J Antimicrob Agents. 36 Suppl 3:S3-7; Goff D A. (2011) Curr Opin Infect Dis. 24 Suppl 1: S11-20; Gould I M. (2010) Int J Antimicrob Agents. 36 Suppl 3: 51-2; Tamma P D, Cosgrove S E. (2011) Infect Dis Clin North Am. 25: 245-260). The use of traditional antibiotics not only select for resistance in a broad range of pathogens, but also disturbs and alters the natural flora, which plays an important role in human and animal health (Guarner F, Malagelada J-R. (2003) Lancet 361: 512-519; Kau A L et al (2011) Nature 474: 327-336; Rashid M U, Weintraub A, Nord C E. (2012) Anaerobe 18: 249-253; Buffle C G, Pamer E G. (2013) Nat Rev Immunol. 2013; 13: 790-801). In particular, in animal and breeding programs, particularly for consumer or human consumption, the use of antibiotics is being increasingly discouraged and efforts are focused to reduce or replace traditional antibiotics with alternative agents or approaches.

Antimicrobial peptides (AMPs) present an alternative to classical antibiotics. AMPs have been found in all kingdoms of life and are part of the innate immunity and represent the first line of defense in an infection (Zasloff M. (2002) Nature 415: 389-95). Despite their diversity in origin and sequence, they generally have a substantial proportion of hydrophobic amino acids (=>30%), an overall positive charge (+2 to +11) and are relatively short consisting of 10⁻⁵⁰ amino acids (Hancock R E W, Sahl H-G (2006) Nat Biotech 24:1551-1557). Based on these properties, AMPs are able to fold into amphiphilic three-dimensional structures and are often based on their secondary structure categorized into α-helical, β-sheet or peptides with extended/random coil structure. Most of the so far characterized AMPs belong to the family of the α-helical or β-sheet peptides (Takahashi D, Shukla S K, Prakash O, Zhang G. (2010) Biochimie pp. 1236±1241; Nguyen I T, Haney E F, Vogel H J. (2011) Trends in Biotechnology pp. 464-472.

CAP18, originally isolated from rabbit neutrophils, demonstrates antimicrobial activity against a broad range of pathogenic bacteria, is highly thermostable and showed no hemolytic activity in vitro (Ebbensgaard A, Mordhorst H, Overgaard M T, Nielsen C G, Aarestrup F M, Hansen E B. (2015) PLoS One 10:e0144611). In addition, a recent study evaluated a potential therapeutic effect of CAP18 against red mouth disease caused by Y. ruckeri in juvenile rainbow trout either by oral administration or intraperitoneal injection, and injection of CAP18 into juvenile rainbow trout before exposure to Y. ruckeri was associated with lower mortality compared to non-treated fish (Chettri J K, Mehrdana F, Hansen E B, Ebbensgaard A, Overgaard M T, Lauritsen A H, et al. (2017) J Fish Dis. 40: 97±104). CAP18 has the potential to act as lead peptide for further development and optimization.

Methane (CH₄) is produced as a by-product of ruminal microbial fermentation process. In particular, H₂ and CO₂ are byproducts of the fermentation process and react to form methane. Methane is generated in the rumen by methanogens, with only a small amount emitted from hind-gut fermentation. Methanogens, unique microbes from Archaeal domain, are responsible for methane production via methanogenesis pathway. They are common in wetlands, where they are responsible for marsh gas, and in the digestive tracts of animals such as ruminants and many humans, where they are responsible for the methane content of belching in ruminants and flatulence in humans. Ruminants are large hoofed herbivorous grazing or browsing mammals that are able to acquire nutrients from plant-based food by fermenting it in a specialized fore-stomach, principally through microbial actions. In marine sediments, the biological production of methane, also termed methanogenesis, is generally confined to where sulfates are depleted, below the top layers. Moreover, methanogenic archaea populations play an indispensable role in anaerobic wastewater treatments. Others are extremophiles, found in environments such as hot springs and submarine hydrothermal vents as well as in the “solid” rock of Earth's crust, kilometers below the surface. Methanogens play important roles in the global carbon cycle (i.e., marine and freshwater ecosystems).

The global population will increase by 33% in 2050 to 9.6 Billion. The projected meat and milk protein demand will rise to 73 and 58%, respectively, compared to those in 2010 (FAO). See Tackling climate change through livestock, FAO, 2013. An increase in livestock productions is expected to make a significant contribution to global climate change (GHG emissions, N₂O, CO₂, CH₄), as livestock GHG have accounted for 18% of global emissions. Among GHG, methane has a shorter shelf life and is 28-times more potent than CO₂. Enteric methane emissions account for 44.3% of GHG emissions from livestock production. Moreover, methane emission is considered loss of energy for animals.

There are net zero initiatives in the cattle industry and market opportunities for the reduction of enteric CH₄ emissions. Net zero pledges from supply chains puts pressure on cattle industries to meet sustainability/greenhouse gas (GHG) reduction goal(s). In addition, a government incentive program providing for a carbon credit can provide an incentive to reduce methane emissions (i.e., President Biden's administration 2030 aggressive goal for reduction of GHG in the US). Thus, there is a need to reduce GHG emissions. In particular, there is a need to reduce methane emissions from livestock.

The citation of references herein shall not be construed as an admission that such is prior art to the presently disclosed subject matter.

### SUMMARY

The present disclosure relates to variant CAP18 peptides having enhanced anti-pathogen activity and improved stability and resistance to protease as candidates and peptides for use and application in controlling, alleviating, or reducing colonization or infection by bacterial parasitic or viral pathogens. Variant CAP18 peptides comprise one or more mutant or modified amino acid that renders the peptides at least as active or more active in killing or inhibiting one or more target pathogen and at least as stable or resistant or more stable or resistant to heat and/or protease. The variant peptides have improved and useful characteristics to provide greater utility and application against one or more pathogens.

The wild type CAP18 sequence and exemplary CAP18 polypeptide variants each having a single amino acid mutation are provided in the following table with variation from the wild type sequence being in bold and underlined.

TABLE 1

| CAP18 | | | SEQ |
|---|---|---|---|
| Peptide or | | | ID |
| Mutant | Mutation Sequence | Mutant type | NO: |
| WT | GLRKRLRKFRNKIKEKLKKIGQKIQGLLPKLAPRTDY | N/A | 1 |
| (AMP01) | | | |
| c1 | GLRPRLRKFRNKIKEKLKKIGQKIQGLLPKLAPRTDY | Proline | 2 |
| C2 | GLRKRLRPFRNKIKEKLKKIGQKIQGLLPKLAPRTDY | Proline | 3 |
| C3 | GLRKRLRKFRPKIKEKLKKIGQKIQGLLPKLAPRTDY | Proline | 4 |
| C4 | GLRKRLRKFRNKIKPKLKKIGQKIQGLLPKLAPRTDY | Proline | 5 |
| C5 | GLRKRLRKFRNKIKEKLKKIGQKIQGLLPKPAPRTDY | Proline | 6 |
| C6 | GLRKRLRKFRNKIKEKLKKIGQKIQGLLPKLAPRPDY | Proline | 7 |
| C7 | GLKKRLRKFRNKIKEKLKKIGQKIQGLLPKLAPRTDY | R→K trypsin | 8 |
| C8 | GLRKKLRKFRNKIKEKLKKIGQKIQGLLPKLAPRTDY | R→K trypsin | 9 |
| C9 | GLRKRLKKFRNKIKEKLKKIGQKIQGLLPKLAPRTDY | R→K trypsin | 10 |
| C10 | GLRKRLRKFKNKIKEKLKKIGQKIQGLLPKLAPRTDY | R→K trypsin | 11 |
| C11 | GLRKRLRKFRNKIKEKLKKIGQKIQGLLPKLAPKTDY | R→K trypsin | 12 |
| C12 | GLRKRLRKIRNKIKEKLKKIGQKIQGLLPKLAPRTDY | F→I | 13 |
| | | Chymotrypsin | |
| C13 | GIRKRLRKFRNKIKEKLKKIGQKIQGLLPKLAPRTDY | L→I | 14 |
| | | Chymotrypsin | |
| C14 | GLRKRIRKFRNKIKEKLKKIGQKIQGLLPKLAPRTDY | L→I | 15 |
| | | Chymotrypsin | |
| C15 | GLRKRLRKFRNKIKEKIKKIGQKIQGLLPKLAPRTDY | L→I | 16 |
| | | Chymotrypsin | |
| C16 | GLRKRLRKFRNKIKEKLKKIGQKIQGLLPKIAPRTDY | L→I | 17 |
| | | Chymotrypsin | |

Exemplary CAP18 variant polypeptides are provided as follows in which mutations or amino acid changes from the wild type CAP18 (AMP01) sequence are shown in n underlined.

>cap18_R2Kv3_NTinh
(SEQ ID NO: 18)
CWTKSIPPKPCGLRKRLKKIKNKIKEKLKKIGQKIQGLLPKLAPRTDY
>cap18_hphobic2_NTinh
(SEQ ID NO: 19)
CWTKSIPPKPCGLRKILKKIKNKIKEKLKKIGQKIQGLLPKLAPRTDY
>cap18_R2Kv2_NTinh
(SEQ ID NO: 20)
CWTKSIPPKPCGLRKKLKKIRNKIKEKLKKIGQKIQGLLPKLAPRTDY
>cap18_R2Kv4_NTinh
(SEQ ID NO: 21)
CWTKSIPPKPCGLRKRLRKIKNKIKEKLKKIGQKIQGLLPKLAPKTDY
>cap18_CRKP1_NTinh
(SEQ ID NO: 22)
CWTKSIPPKPCGCRKPLRKIRNKIKEKLKKIGQKIQGLLPKLAPRTDY
>cap18_CRKP2_NTinh
(SEQ ID NO: 23)
CWTKSIPPKPCGCRKPCRKIRNKIKEKLKKIGQKIQGLLPKLAPRTDY

Exemplary variant CAP18 peptides are provided as follows without the N terminal CWTKSIPPKPC sequences in which mutations or amino acid changes from the wild type CAP18 (AMP01) sequence are shown in bold and underlined.

>cap18_R2Kv3_NTinh no CWT N term
(SEQ ID NO: 24)
GLRKRLKKIKNKIKEKLKKIGQKIQGLLPKLAPRTDY
>cap18_hphobic2_NTinh no CWT N term
(SEQ ID NO: 25)
GLRKILKKIKNKIKEKLKKIGQKIQGLLPKLAPRTDY
>cap18_R2Kv2_NTinh no CWT N term
(SEQ ID NO: 26)
GLRKKLKKIRNKIKEKLKKIGQKIQGLLPKLAPRTDY
>cap18_R2Kv4_NTinh no CWT N term
(SEQ ID NO: 27)
GLRKRLRKIKNKIKEKLKKIGQKIQGLLPKLAPKTDY
>cap18_CRKP1_NTinh no CWT N term
(SEQ ID NO: 28)
GCRKPLRKIRNKIKEKLKKIGQKIQGLLPKLAPRTDY
>cap18_CRKP2_NTinh no CWT N term
(SEQ ID NO: 29)
GCRKPCRKIRNKIKEKLKKIGQKIQGLLPKLAPRTDY

In other embodiments, exemplary CAP18 variant peptides are provided with significant trypsin resistance, particularly as compared to wild type CAP18 (AMP01) sequence. These exemplary variant CAP18 polypeptides are as follows in which mutations or amino acid changes from the wild type CAP18 (AMP01) sequence are shown in bold and underlined.

>cap18_NTinh
(SEQ ID NO: 30)
CWTKSIPPKPCGLRKRLRKIRNKIKEKLKKIGQKIQGLLPKLAPRTDY
>cap18_R2Kv3
(SEQ ID NO: 31)
GLRKRLKKIKNKIKEKLKKIGQKIQGLLPKLAPRTDY
>cap18_hphobic2
(SEQ ID NO: 32)
GLRKILKKIKNKIKEKLKKIGQKIQGLLPKLAPRTDY
>cap18_R2Kv2
(SEQ ID NO: 33)
GLRKKLKKIRNKIKEKLKKIGQKIQGLLPKLAPRTDY
>cap18_R2Kv4
(SEQ ID NO: 34)
GLRKRLRKIKNKIKEKLKKIGQKIQGLLPKLAPKTDY
>cap18_CRKP1
(SEQ ID NO: 35)
GCRKPLRKIRNKIKEKLKKIGQKIQGLLPKLAPRTDY
>cap18_CRKP2
(SEQ ID NO: 36)
GCRKPCRKIRNKIKEKLKKIGQKIQGLLPKLAPRTDY

In other embodiments, exemplary CAP18 variant peptides are provided with moderate but improved trypsin resistance, particularly as compared to wild type CAP18 (AMP01) sequence. These exemplary variant CAP18 polypeptides are provided as follows in which mutations or amino acid changes from the wild type CAP18 (AMP01) sequence are shown in bold and underlined.

>cap18_R2Kv6
(SEQ ID NO: 37)
GLRKKLKKIKNKIKEKLKKIGQKIQGLLPKLAPRTDY
>cap18_R2Kv7
(SEQ ID NO: 38)
GLKKKLKKIKNKIKEKLKKIGQKIQGLLPKLAPRTDY
>cap18_R2Kv8
(SEQ ID NO: 39)
GLKKKLKKIKNKIKEKLKKIGQKIOGLLPKLAPKTDY
>cap18_hphobic1
(SEQ ID NO: 40)
GLRKILRKIRNKIKEKLKKIGQKIQGLLPKLAPRTDY
>cap18_hphobic3
(SEQ ID NO: 41)
GLKKILKKIKNKIKEKLKKIGQKIQGLLPKLAPRTDY
>cap18_R2Kv1
(SEQ ID NO: 42)
GLKKKLRKIRNKIKEKLKKIGQKIQGLLPKLAPRTDY

Some CAP18 variants did not evidence activity or protease resistance which was comparable at least to the wild type CAP18 peptide. Exemplary CAP18 polypeptides include alternative N terminal or C terminal tags or additional sequence (shown in bold) with a comparison of variant amino acid sequence with wild type sequence being shown in bold and underlined.

>cap18_cyc1
(SEQ ID NO: 43)
CGGGLRKRLRKIRNKIKEKLKKIGQKIQGLLPKLAPRTDYGGC
>cap18_cyc2
(SEQ ID NO: 44)
CGGSGLRKRLRKIRNKIKEKLKKIGQKIQGLLPKLAPRTDYGSGGC
>cap18_cycdimer
(SEQ ID NO: 45)
CGGGLRKRLRKIRNKIKEKLKKIGQKIQGLLPKLAPRTDYGAGGGLRKR
LRKFRNKIKEKLKKIGQKIQGLLPKLAPRTDYGGC
>cap18_NTinh
(SEQ ID NO: 46)
CWTKSIPPKPCGLRKRLRKIRNKIKEKLKKIGQKIQGLLPKLAPRTDY
>cap18_NTCTinh
(SEQ ID NO: 47)
CWTKSIPPKPCGLRKRLRKIRNKIKEKLKKIGQKIQGLLPKLAPRTDYC
WTKSIPPKPC
>cap18_CRKP3
(SEQ ID NO: 48)
GCRKPCRKPRNKIKEKLKKIGQKIQGLLPKLAPRTDY

Another exemplary engineered polypeptide comprises:

(SEQ ID NO: 49)
GLRKRLRKIRNKIKEKLKKIGQKIQGLLPKLAPRTDYCWTKSIPPKPC.

CAP18 is in the cathelicidin family of antimicrobial polypeptides and engineered polypeptide variants of the wild type CAP18 rabbit polypeptide are disclosed herein. Other antimicrobial cathelicidins including engineered polypeptide variants of different species that are disclosed herein include BMAP28 (CATHL5; bovine), Bac7 (CATHL3; bovine rumen), k9Cath (canine) and PMAP36 (porcine). These polypeptides are not homologues of CAP18. BMAP is bovine myeloid antimicrobial peptide. PMAP is porcine myeloid antimicrobial peptide. Bac refers to bactenecin antimicrobial peptides. The other engineered cathelicidin polypeptides provided herein may have protease resistance, particularly as compared to the wild type cathelicidin sequences. These exemplary other cathelicidin polypeptides are provided as follows in which alternative N terminal or C terminal tags or additional sequence are shown in bold with a comparison of variant amino acid sequence with wild type sequence being shown in bold and underlined.

### Engineered BMAP28 Polypeptides:

>BMAP28_WT
(SEQ ID NO: 50)
GGLRSLGRKILRAWKKYGPIIVPIIRIG;
>BMAP28_NTinh
(SEQ ID NO: 51)
CWTKSIPPKPCGGLRSLGRKILRAWKKYGPIIVPIIRIG;
>BMAP28_NTinh_trunc:
(SEQ ID NO: 52)
CWTKSIPPKPCGGLRSLGRKILRAWKKYG;
>BMAP28_NTinh2
(SEQ ID NO: 53)
CRKPGGLRSLGRKILRAWKKYGPIIVPIIRIG;
>BMAP28_NTinh2_trunc
(SEQ ID NO: 54)
CRKPGGLRSLGRKILRAWKKYG;
>BMAP28_R2K1
(SEQ ID NO: 55)
GGLKSLGKKILRAWKKYGPIIVPIIRIG;
>BMAP28_R2K2
(SEQ ID NO: 56)
GGLRSLGKKILKAWKKYGPIIVPIIRIG;
>BMAP28_R2K3
(SEQ ID NO: 57)
GGLRSLGRKILKAWKKYGPIIVPIIKIG;
>BMAP28_R2K4
(SEQ ID NO: 58)
GGLKSLGKKILKAWKKYGPIIVPIIKIG;
>BMAP28_hydrophobic1
(SEQ ID NO: 59)
GGLRSLGRKILRAIKKYGPIIVPIIRIG;
>BMAP28_hydrophobic2
(SEQ ID NO: 60)
GGLRSLGRKILRAWKKIGPIIVPIIRIG;
>BMAP28_hydrophibic3
(SEQ ID NO: 61)
GGLRSLGRKILRAIKKIGPIIVPIIRIG;
>BMAP28_hydrophobic4
(SEQ ID NO: 62)
GGARSLGRKALRAAKKAGPAIVPIIRIG.

### Engineered BAC7 Polypeptides:

>Bac7_WT
(SEQ ID NO: 63)
RRIRPRPPRLPRPRPRPLPFPRPGPRPIPRPLPFPRPGPRPIPRPLPFP
RPGPRPIPRPL;
>Bac7_NTinh1
(SEQ ID NO: 64)
CWTKSIPPKPCRRIRPRPPRLPRPRPRPLPFPRPGPRPIPRPLPFPRPG
PRPIPRPLPFPRPGPRPIPRPL;
>Bac7_NTinh2
(SEQ ID NO: 65)
CRKPRRIRPRPPRLPRPRPRPLPFPRPGPRPIPRPLPFPRPGPRPIPRP
LPFPRPGPRPIPRPL;
>Bac7_trunc
(SEQ ID NO: 66)
RRIRPRPPRLPRPRPR;
>Bac7_NTinh1_trunc
(SEQ ID NO: 67)
CWTKSIPPKPCRRIRPRPPRLPRPRPR;
>Bac7_NTinh2_trunc
(SEQ ID NO: 68)
CRKPRRIRPRPPRLPRPRPR;
>Bac7_NTinh2
(SEQ ID NO: 69)
CRKPRRIRPRPPRLPRPRPRPLPFPRPGPRPIPRPLPFPRPGPRPIPRP
LPFPRPGPRPIPRPL;
>Bac7_hydrophobic1
(SEQ ID NO: 70)
RRIRPRPPRLPRPRPRPLPIPRPGPRPIPRPLPIPRPGPRPIPRPLPFP
RPGPRPIPRPL;
>Bac7_hydrophobic2
(SEQ ID NO: 71)
RRIRPRPPRLPRPRPRPLPFPRPGPRPIPRPLPIPRPGPRPIPRPLPIP
RPGPRPIPRPL;
>Bac7_hydrophobic3
(SEQ ID NO: 72)
RRIRPRPPRLPRPRPRPLPIPRPGPRPIPRPLPIPRPGPRPIPRPLPIP
RPGPRPIPRPL;
>Bac7_R2K1
(SEQ ID NO: 73)
KKIRPRPPRLPRPRPRPLPFPRPGPRPIPRPLPFPRPGPRPIPRPLPFP
RPGPRPIPRPL;
>Bac7_R2K2
(SEQ ID NO: 74)
RKIRPRPPKLPRPRPRPLPFPRPGPRPIPRPLPFPRPGPRPIPRPLPFP
RPGPRPIPRPL;
>Bac7_R2K3
(SEQ ID NO: 75)
KKIRPRPPKLPRPRPRPLPFPRPGPRPIPRPLPFPRPGPRPIPRPLPFP
RPGPRPIPRPL;
>Bac7_R2K3trunc
(SEQ ID NO: 76)
KKIRPRPPKLPRPRPR.

### Engineered K9CATH Polypeptides:

>k9Cath_WT
(SEQ ID NO: 77)
RLKELITTGGQKIGEKIRRIGQRIKDFFKNLQPREEKS;
>k9Cath_trunc
(SEQ ID NO: 78)
RLKELITTGGQKIGEKIRRIG;
>k9Cath_WT_Ninh1
(SEQ ID NO: 79)
CWTKSIPPKPCRLKELITTGGQKIGEKIRRIGQRIKDFFKNLQPREEKS;
>k9Cath_trunc_Ninh1
(SEQ ID NO: 80)
CWTKSIPPKPCRLKELITTGGQKIGEKIRRIG;
>k9Cath_WT_Ninh2
(SEQ ID NO: 81)
CRKPRLKELITTGGQKIGEKIRRIGQRIKDFFKNLQPREEKS;
>k9Cath_trunc_Ninh2
(SEQ ID NO: 82)
CRKPRLKELITTGGQKIGEKIRRIG;
>k9Cath_F21
(SEQ ID NO: 83)
RLKELITTGGQKIGEKIRRIGQRIKDIIKNLOPREEKS;
>k9Cath_R2K1
(SEQ ID NO: 84)
KLKELITTGGQKIGEKIKRIGQRIKDFFKNLQPREEKS;
>k9Cath_R2K2
(SEQ ID NO: 85)
RLKELITTGGQKIGEKIKKIGQRIKDFFKNLQPREEKS;
>k9Cath_R2K3
(SEQ ID NO: 86)
RLKELITTGGQKIGEKIRKIGQRIKDFFKNLQPKEEKS;
>k9Cath_R2K4
(SEQ ID NO: 87)
RLKELITTGGQKIGEKIKKIGQRIKDFFKNLQPKEEKS;
>k9Cath_trunc_R2K4
(SEQ ID NO: 88)
RLKELITTGGQKIGEKIKKIG;
>k9Cath_trunc_R2K4_Ninh1
(SEQ ID NO: 89)
CWTKSIPPKPCRLKELITTGGQKIGEKIKKIG

### Engineered PMAP36 Polypeptides:

>PMAP36_WT
(SEQ ID NO: 90)
GRFRRLRKKTRKRLKKIGKVLKWIPPIVGSIPLGCG;
>PMAP36_Ninh1
(SEQ ID NO: 91)
CWTKSIPPKPCGRFRRLRKKTRKRLKKIGKVLKWIPPIVGSIPLGCG;
>PMAP36_Ninh2
(SEQ ID NO: 92)
CRKPGRFRRLRKKTRKRLKKIGKVLKWIPPIVGSIPLGCG;
>PMAP36_trunc
(SEQ ID NO: 93)
GRFRRLRKKTRKRLKKIGKVLKWI;
>PMAP36_trunc_Ninh1
(SEQ ID NO: 94)
CWTKSIPPKPCGRFRRLRKKTRKRLKKIGKVLKWI;
>PMAP36_trunc_Ninh2
(SEQ ID NO: 95)
CRKPGRFRRLRKKTRKRLKKIGKVLKWI;
>PMAP36_trunc_W2L
(SEQ ID NO: 96)
GRFRRLRKKTRKRLKKIGKVLKLI;
>PMAP36_trunc_WF2L
(SEQ ID NO: 97)
GRLRRLRKKTRKRLKKIGKVLKLI;
>PMAP36_trunc_6V_W2L
(SEQ ID NO: 98)
GVFRVLRKVTRVVLKVIGKVLKLI;
>PMAP36_trunc_6V_WF2L
(SEQ ID NO: 99)
GVLRVLRKVTRVVLKVIGKVLKLI;
>PMAP36_trunc2
(SEQ ID NO: 100)
RRLRKKTRKRLKKIGKVLKWI;
>PMAP36_trunc2_Ninh
(SEQ ID NO: 101)
CWTKSIPPKPCRRLRKKTRKRLKKIGKVLKWI;
>PMAP36_trunc2_Ninh_R2K_W2L
(SEQ ID NO: 102)
CWTKSIPPKPCRKLRKKTRKRLKKIGKVLKLI.

In an embodiment, the present disclosure provides an engineered polypeptide variant of CAP18, the polypeptide including:

(SEQ ID NO: 103)
X₁X₂X₃KX₄X₅X₆KX₇X₈NKIKEKLKKIGQKIQGLLPKLAPX₉TDX₁₀,

wherein X1 Is G or CWTKSIPPKPC-G (SEQ ID NO: 104),
X2 is C or L,
X3 is R or K,
X4 is P, A, V, I, L, M, F, Y, or W,
X5 is C or L,
X6 is R or K,
X7 is I or K,
X8 is R, I, or K,
X9 is R or K,
X10 Is Y or Y-CWTKSIPPKPC (SEQ ID NO: 105), and
wherein the polypeptide does not include GLRKRLRKFRNKIKEKLKKIGQKIQGLLPKLAPRTDY (SEQ ID NO: 1).

In an embodiment, the engineered polypeptide variant of CAP18 includes one of the following:

SEQ ID NO: 13)
GLRKRLRKIRNKIKEKLKKIGQKIQGLLPKLAPRTDY;
(SEQ ID NO: 49)
GLRKRLRKIRNKIKEKLKKIGQKIQGLLPKLAPRTDYCWTKSIPPKPC;
(SEQ ID NO: 46)
CWTKSIPPKPCGLRKRLRKIRNKIKEKLKKIGQKIQGLLPKLAPRTDY;
(SEQ ID NO: 47)
CWTKSIPPKPCGLRKRLRKIRNKIKEKLKKIGQKIQGLLPKLAPRTDYC
WTKSIPPKPC;
(SEQ ID NO: 18)
CWTKSIPPKPCGLRKRLKKIKNKIKEKLKKIGQKIQGLLPKLAPRTDY;
(SEQ ID NO: 19)
CWTKSIPPKPCGLRKILKKIKNKIKEKLKKIGQKIQGLLPKLAPRTDY;
(SEQ ID NO: 20)
CWTKSIPPKPCGLRKKLKKIRNKIKEKLKKIGQKIQGLLPKLAPRTDY;
(SEQ ID NO: 21)
CWTKSIPPKPCGLRKRLRKIKNKIKEKLKKIGQKIQGLLPKLAPKTDY;
(SEQ ID NO: 22)
CWTKSIPPKPCGCRKPLRKIRNKIKEKLKKIGQKIQGLLPKLAPRTDY;
(SEQ ID NO: 23)
CWTKSIPPKPCGCRKPCRKIRNKIKEKLKKIGQKIQGLLPKLAPRTDY;
(SEQ ID NO: 30)
CWTKSIPPKPCGLRKRLRKIRNKIKEKLKKIGQKIQGLLPKLAPRTDY;
(SEQ ID NO: 31)
GLRKRLKKIKNKIKEKLKKIGQKIQGLLPKLAPRTDY;
(SEQ ID NO: 32)
GLRKILKKIKNKIKEKLKKIGQKIQGLLPKLAPRTDY;
(SEQ ID NO: 33)
GLRKKLKKIRNKIKEKLKKIGQKIQGLLPKLAPRTDY;
(SEQ ID NO: 34)
GLRKRLRKIKNKIKEKLKKIGQKIQGLLPKLAPKTDY;
(SEQ ID NO: 35)
GCRKPLRKIRNKIKEKLKKIGQKIQGLLPKLAPRTDY;
(SEQ ID NO: 36)
GCRKPCRKIRNKIKEKLKKIGQKIQGLLPKLAPRTDY;
(SEQ ID NO: 37)
GLRKKLKKIKNKIKEKLKKIGQKIQGLLPKLAPRTDY;
(SEQ ID NO: 38)
GLKKKLKKIKNKIKEKLKKIGQKIQGLLPKLAPRTDY;
(SEQ ID NO: 39)
GLKKKLKKIKNKIKEKLKKIGQKIQGLLPKLAPKTDY;
(SEQ ID NO: 40)
GLRKILRKIRNKIKEKLKKIGQKIQGLLPKLAPRTDY;
(SEQ ID NO: 41)
GLKKILKKIKNKIKEKLKKIGQKIQGLLPKLAPRTDY;
or
(SEQ ID NO: 42)
GLKKKLRKIRNKIKEKLKKIGQKIQGLLPKLAPRTDY.

In another embodiment, the disclosure provides the following engineered polypeptide variant of CAP18:

(SEQ ID NO: 106)
GX₁RKX₂X₃X₄KX₅X₆NKIKEKLKKIGQKIQGLLPKLAPX₇TDY,

wherein X1 is C or L,
X2 is R, K, I, or P,
X3 is I or C,
X4 is I or R,
X5 is I,
X6 is R or K,
X7 is R or K.

In an embodiment, the engineered polypeptide variant of CAP18 includes one of the following:

(SEQ ID NO: 30)
CWTKSIPPKPCGLRKRLRKIRNKIKEKLKKIGQKIQGLLPKLAPRTDY;
(SEQ ID NO: 31)
GLRKRLKKIKNKIKEKLKKIGQKIQGLLPKLAPRTDY;
(SEQ ID NO: 32)
GLRKILKKIKNKIKEKLKKIGQKIQGLLPKLAPRTDY;
(SEQ ID NO: 33)
GLRKKLKKIRNKIKEKLKKIGQKIQGLLPKLAPRTDY;
(SEQ ID NO: 34)
GLRKRLRKIKNKIKEKLKKIGQKIQGLLPKLAPKTDY;
(SEQ ID NO: 35)
GCRKPLRKIRNKIKEKLKKIGQKIQGLLPKLAPRTDY;
or
(SEQ ID NO: 36)
GCRKPCRKIRNKIKEKLKKIGQKIQGLLPKLAPRTDY.

In an embodiment, the present disclosure provides an engineered polypeptide variant of BMAP28, the polypeptide including:

(SEQ ID NO: 107)
X₁GX₂X₃SLGX₄KX₅LX₆AX₇KKX₈GPX₉IVPIIX₁₀IG,

wherein X1 is G, CWTKSIPPKPC-G (SEQ ID NO: 104) or CRKP-G (SEQ ID NO: 108),
X2 is I or A,
X3 is R or K,
X4 is R or K,
X5 is I or A,
X6 is R or K,
X7 is W, I or A,
X8 is Y, I or A,
X9 is I or A, and
X10 Is R or K,
wherein the polypeptide does not include GGLRSLGRKILRAWKKYGPIIVPIIRIG (SEQ ID NO: 50).

In an embodiment, the engineered polypeptide variant of BMAP28 includes one of the following:

(SEQ ID NO: 51)
CWTKSIPPKPCGGLRSLGRKILRAWKKYGPIIVPIIRIG;
(SEQ ID NO: 53)
CRKPGGLRSLGRKILRAWKKYGPIIVPIIRIG;
(SEQ ID NO: 55)
GGLKSLGKKILRAWKKYGPIIVPIIRIG;
(SEQ ID NO: 56)
GGLRSLGKKILKAWKKYGPIIVPIIRIG;
(SEQ ID NO: 57)
GGLRSLGRKILKAWKKYGPIIVPIIKIG;
(SEQ ID NO: 58)
GGLKSLGKKILKAWKKYGPIIVPIIKIG;
(SEQ ID NO: 59)
GGLRSLGRKILRAIKKYGPIIVPIIRIG;
(SEQ ID NO: 60)
GGLRSLGRKILRAWKKIGPIIVPIIRIG;
(SEQ ID NO: 61)
GGLRSLGRKILRAIKKIGPIIVPIIRIG;
or
(SEQ ID NO: 62)
GGARSLGRKALRAAKKAGPAIVPIIRIG.

In an embodiment, the present disclosure provides an engineered polypeptide, truncated BMAP28, the polypeptide including:

(SEQ ID NO: 109)
XGLRSLGRKILRAWKKYG,

wherein X is G, CWTKSIPPKPC-G (SEQ ID NO: 104), or CRKP-G (SEQ ID NO: 108).
In an embodiment, the present disclosure provides an engineered polypeptide variant of BAC7, the polypeptide including:

(SEQ ID NO: 110)
X₁X₂IRPRPPX₃LPRPRPRPLPX₄PRPGPRPIPRPLPX₅PRPGPRPIPRPL
PX₆PRPGPRPIPRPL,

wherein X1 is R, CWTKSIPPKPC-R (SEQ ID NO: 111), CRKP-R (SEQ ID NO: 112) or K,
X2 Is R or K,
X3 Is R or K,
X4 is F or I,
X5 Is F or I, and
X6 is F or I,
wherein the polypeptide does not include

(SEQ ID NO: 63)
RRIRPRPPRLPRPRPRPLPFPRPGPRPIPRPLPFPRPGPRPIPRPLPFP
RPGPRPIPRPL.

In an embodiment, the engineered polypeptide variant of BAC7 Includes one of the following:

(SEQ ID NO: 64)
CWTKSIPPKPCRRIRPRPPRLPRPRPRPLPFPRPGPRPIPRPLPFPRPG
PRPIPRPLPFPRPGPRPIPRPL;
(SEQ ID NO: 65)
CRKPRRIRPRPPRLPRPRPRPLPFPRPGPRPIPRPLPFPRPGPRPIPRP
LPFPRPGPRPIPRPL;
(SEQ ID NO: 70)
RRIRPRPPRLPRPRPRPLPIPRPGPRPIPRPLPIPRPGPRPIPRPLPFP
RPGPRPIPRPL;
(SEQ ID NO: 71)
RRIRPRPPRLPRPRPRPLPFPRPGPRPIPRPLPIPRPGPRPIPRPLPIP
RPGPRPIPRPL;
(SEQ ID NO: 72)
RRIRPRPPRLPRPRPRPLPIPRPGPRPIPRPLPIPRPGPRPIPRPLPIP
RPGPRPIPRPL;
(SEQ ID NO: 73)
KKIRPRPPRLPRPRPRPLPFPRPGPRPIPRPLPFPRPGPRPIPRPLPFP
RPGPRPIPRPL;
(SEQ ID NO: 74)
RKIRPRPPKLPRPRPRPLPFPRPGPRPIPRPLPFPRPGPRPIPRPLPFP
RPGPRPIPRPL;
or
(SEQ ID NO: 75)
KKIRPRPPKLPRPRPRPLPFPRPGPRPIPRPLPFPRPGPRPIPRPLPFP
RPGPRPIPRPL.

In an embodiment, the present disclosure provides an engineered polypeptide, truncated BAC7, the polypeptide including:

(SEQ ID NO: 113)
X₁X₂IRPRPPX₃LPRPRPR,

wherein X1 is R, CWTKSIPPKPC-R (SEQ ID NO: 111) or CRKP-R (SEQ ID NO: 112),
X2 is R or K, and
X3 is R or K.

In an embodiment, the engineered polypeptide, truncated BAC7 includes one of the following:

(SEQ ID NO: 66)
RRIRPRPPRLPRPRPR;
(SEQ ID NO: 67)
CWTKSIPPKPCRRIRPRPPRLPRPRPR;
(SEQ ID NO: 68)
CRKPRRIRPRPPRLPRPRPR;
or
(SEQ ID NO: 76)
KKIRPRPPKLPRPRPR.

In an embodiment, the present disclosure provides an engineered polypeptide variant of K9CATH, the polypeptide including:

(SEQ ID NO: 114)
X₁LKELITTGGQKIGEKIX₂X₃IGQRIKDX₄X₅KNLQPX₆EEKS,

wherein X1 is R, CWTKSIPPKPC-R (SEQ ID NO: 111), CRKP-R (SEQ ID NO: 112) or K,
X2 is R or K,
X3 is R or K,
X4 is F or I,
X5 is F or I, and
X6 is R or K,
wherein the polypeptide does not include

(SEQ ID NO: 77)
RLKELITTGGQKIGEKIRRIGQRIKDFFKNLQPREEKS.

In an embodiment, the engineered polypeptide of K9CATH includes one of the following:

(SEQ ID NO: 83)
RLKELITTGGQKIGEKIRRIGQRIKDIIKNLQPREEKS;
(SEQ ID NO: 84)
KLKELITTGGQKIGEKIKRIGQRIKDFFKNLQPREEKS;
(SEQ ID NO: 85)
RLKELITTGGQKIGEKIKKIGQRIKDFFKNLQPREEKS;
(SEQ ID NO: 86)
RLKELITTGGQKIGEKIRKIGQRIKDFFKNLQPKEEKS;
or
(SEQ ID NO: 87)
RLKELITTGGQKIGEKIKKIGQRIKDFFKNLQPKEEKS.

In an embodiment, the present disclosure provides an engineered polypeptide, truncated K9CATH, the polypeptide including:

(SEQ ID NO: 115)
X₁LKELITTGGQKIGEKIX₂X₃IG,

wherein X1 Is R, CWTKSIPPKPC-R (SEQ ID NO: 111) or CRKP-R (SEQ ID NO: 112),
X2 is R or K, and
X3 Is R or K.

In an embodiment, the engineered polypeptide, truncated K9CATH includes one of the following:

(SEQ ID NO: 88)
RLKELITTGGQKIGEKIKKIG;
or
(SEQ ID NO: 89)
CWTKSIPPKPCRLKELITTGGQKIGEKIKKIG.

In an embodiment, the present disclosure provides an engineered polypeptide, truncated PMAP36, the polypeptide including:

(SEQ ID NO: 116)
X₁X₂X₃RX₄LRKX₅TRX₆X₇LKX₈IGKVLKX₉I,

wherein X1 is G, CWTKSIPPKPC-G (SEQ ID NO:104) or CRKP-G (SEQ ID NO: 108),
X2 is R or V,
X3 is F or L,
X4 is R or V,
X5 is K or V,
X6 is K or V,
X7 is R or V,
X8 is K or V, and
X9 is W or L

In an embodiment, the engineered polypeptide, truncated PMAP36 includes one of the following:

(SEQ ID NO: 96)
GRFRRLRKKTRKRLKKIGKVLKLI;
(SEQ ID NO: 97)
GRLRRLRKKTRKRLKKIGKVLKLI;
(SEQ ID NO: 98)
GVFRVLRKVTRVVLKVIGKVLKLI;
or
(SEQ ID NO: 99)
GVLRVLRKVTRVVLKVIGKVLKLI

In an embodiment, the present disclosure provides an engineered polypeptide, truncated 2 of PMAP36, the polypeptide including:

(SEQ ID NO: 117)
X₁X₂LRKKTRKRLKKIGKVLKX₃I,

wherein X1 is R or CWTKSIPPKPC-R (SEQ ID NO: 111),
X2 is R or K, and
X3 is W or L

In an embodiment, the engineered polypeptide, truncated 2 PMAP36 includes:

(SEQ ID NO: 102)
CWTKSIPPKPCRKLRKKTRKRLKKIGKVLKLI.

In an embodiment an antimicrobial composition comprises: one or more engineered polypeptide according to the present disclosure; and a pharmaceutically acceptable carrier.

In an embodiment the engineered antimicrobial polypeptide is conjugated or attached to other molecules or agents comprising at least one of peptides conjugated to a cell or pathogen targeting agent or sequence, toxin, immunomodulator, cytokine, cytotoxic agent, or one or more anti-bacterial, anti-parasitic or anti-viral agent or drug.

An embodiment comprises administering the engineered antimicrobial polypeptide in combination with another agent comprising at least one of an anti-bacterial agent, anti-infective agent, and an immunomodulatory agent.

An embodiment provides administering the engineered antimicrobial polypeptide along with or coadministering with one or more prebiotic.

An embodiment comprises administering the engineered antimicrobial polypeptide as part of a composition comprising at least one of animal feed, a feed additive, a food ingredient, a water additive, a water-mixed additive, a consumable solution, a consumable spray additive, a consumable solid, a consumable gel, an injectable, or combinations thereof.

In an embodiment the engineered antimicrobial polypeptide is administered orally or by injection.

In an embodiment the engineered antimicrobial polypeptide is administered as part of a pharmaceutical composition for oral administration in a tablet, a capsule, a powder or a liquid form, the pharmaceutical composition comprising a pharmaceutically acceptable carrier.

In an embodiment the engineered antimicrobial polypeptide is administered as part of a composition including one or more biologically active molecule or therapeutic molecule comprising at least one of an ionophore; a vaccine; an antibiotic; an antihelmintic; a virucide; a nematicide; amino acids including methionine, glycine, or arginine; fish oil; krill oil; and enzymes.

An embodiment includes an antimicrobial composition, the composition comprising an engineered antimicrobial polypeptide as provided herein and a pharmaceutically acceptable carrier. In an embodiment, a pharmaceutical composition Is provided, the composition comprising an engineered antimicrobial polypeptide as provided herein and a pharmaceutically acceptable carrier. In an embodiment, a pharmaceutical composition Is provided, the composition comprising one or more engineered antimicrobial polypeptide as provided herein, one or more antipathogenic agent or immunomodulatory agent, and a pharmaceutically acceptable carrier.

In another embodiment, a method of treating a microbial infection is provided, the method comprising administering to a subject in need thereof, a composition comprising an engineered antimicrobial polypeptide as provided herein.

In an embodiment, the microbial Infection is caused by Mannheimia haemolytica (BRD, cattle), Pasteurella multocida (BRD, cattle), E. coli (Colibacillosis, poultry), Salmonella (Salmonellosis, poultry), C. Jejuni (Campylobacteriosis, poultry), or P. salmonis (SRS, salmon).

In an embodiment, the disclosure includes a method of treating a parasitic infection, the method comprising administering to a subject in need thereof, a composition comprising an engineered antimicrobial polypeptide as provided herein.

In an embodiment, the parasitic infection is caused by Giardia or Eimeria parasites.

In another embodiment, the disclosure includes a method of treating a viral infection, the method comprising administering to a subject in need thereof, a composition comprising an engineered antimicrobial polypeptide as provided herein.

In an embodiment, the viral infection is caused by a virus that infects one or more livestock, poultry or aquatic species of animal.

In an embodiment, the viral infection is caused by PRRSV in swine.

The present disclosure provides compositions and methods for reducing greenhouse gas emissions, particularly methane emissions from animals. The present disclosure relates to antimicrobial peptides (AMP) including the one or more engineered antimicrobial peptide disclosed herein and compositions thereof. The methods for reducing greenhouse gas emissions, particularly methane emissions, use such engineered antimicrobial peptide and compositions.

The present disclosure provides a composition including at least one antimicrobial peptide, wherein the composition reduces methane gas emissions from a ruminant when an effective amount is administered to the ruminant, as compared to a ruminant not administered the composition.

The present disclosure provides a method for reducing methane gas emissions from a ruminant including administering an effective amount of a composition including at least one antimicrobial peptide, or combinations thereof, to a ruminant.

A method comprises administering one or more engineered antimicrobial peptide of the present disclosure to an animal in an amount effective to inhibit growth of at least one methanogen in the animal.

In an embodiment, the methanogen includes at least one of Methanobrevibacter ruminantium DSM 1093, Methanosphaera stadtmanae DSM 3091, Methanomicrobium mobile DSM 1539, Methanobacterium bryantii, Methanobrevibacter gottchackii, Methanobrevibacter olleyae, Methanobrevibacter thauerii, Methanomassilicoccus luminyensis or Methanosarcina barkeri, and combinations thereof.

In an embodiment the engineered antimicrobial polypeptide targets cell membranes of the at least one methanogen, the at least one methanogen being located in the gastrointestinal tract of an animal.

In embodiments of the present disclosure the animal comprises one or more of: cattle which include cows, bulls and calves; poultry which includes broilers, chickens and turkeys; pigs which include piglets; birds; aquatic animals which include fish, agastric fish, gastric fish, freshwater fish which include salmon, cod, trout and carp, marine fish which include salmon and sea bass, and crustaceans which include shrimps, mussels and scallops; horses which include race horses; and sheep which include lambs.

In an embodiment the animal is a ruminant comprising at least one of extensive beef cattle, intensive beef cattle and dairy cattle.

In an embodiment the administration is effective in reducing enteric methane gas emissions from the ruminant in an amount of at least 20%, 30%, 40%, 50% or 60%.

A method of the present disclosure includes administering to an animal a unicellular host capable of heterologously expressing at least one of the engineered polypeptides disclosed herein.

In an embodiment the unicellular host is transformed by a vector that comprises nucleic acid encoding the engineered polypeptide.

In an embodiment the unicellular host includes a genome into which heterologous nucleic acid encoding the engineered polypeptide has been Integrated.

In an embodiment the nucleic acid comprises a recombinant DNA molecule, a recombinant nucleic acid, or cloned gene, or a degenerate variant thereof, encoding the engineered polypeptide.

In an embodiment the unicellular host is administered to the animal by intranasal spray, by injection, as part of a direct fed microbial, or by oral administration.

Other objects and advantages will become apparent to those skilled in the art from a review of the ensuing detailed description, which proceeds with reference to the following illustrative drawings, and the attendant claims.

### BRIEF DESCRIPTION OF DRAWINGS

FIGS. 1A and 18. (A) Schematic representation of CAP18 full length native polypeptide, with signal peptide N terminal region, cathelin domain (about 101-105 amino acids) and LL-37 which is the designated name for the active C terminal peptide or CAP18 peptide. (B) depicts a comparison of the wild type or native rabbit CAP18 peptide sequence and the human CAP18 peptide sequence.

FIG. 2A shows plate inhibition assays of CAP18 (AMP01) peptide against bacteria C. jejuni (Campylobacter), S. typhimurium (Salmonella), and L. reuteri (Lactobacillus).

FIG. 28 shows plate assay inhibition of BRD pathogens M. haemolytica and P. mutlocida (MIC of 4-8 μg/ml).

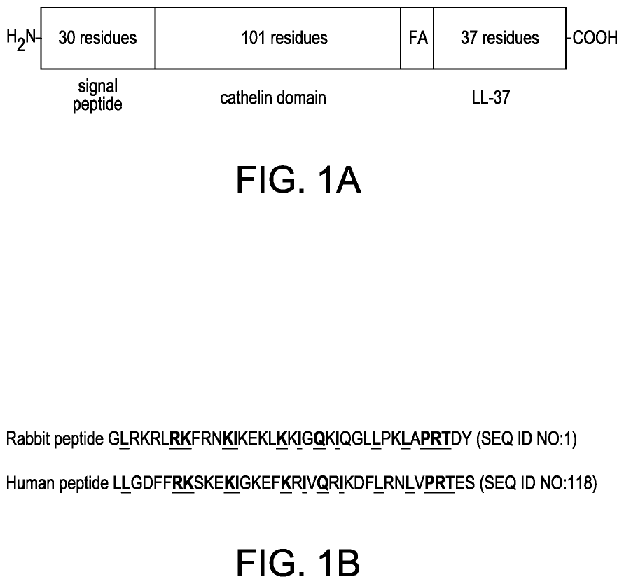

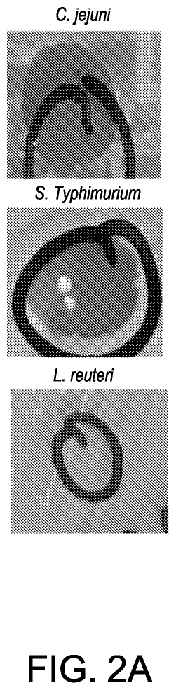

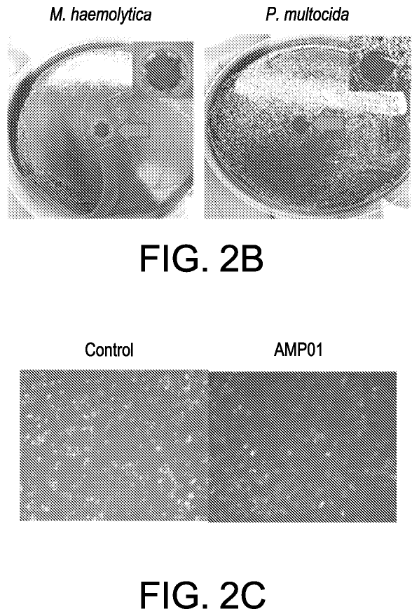

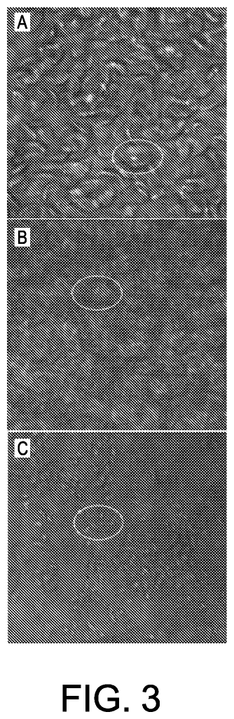

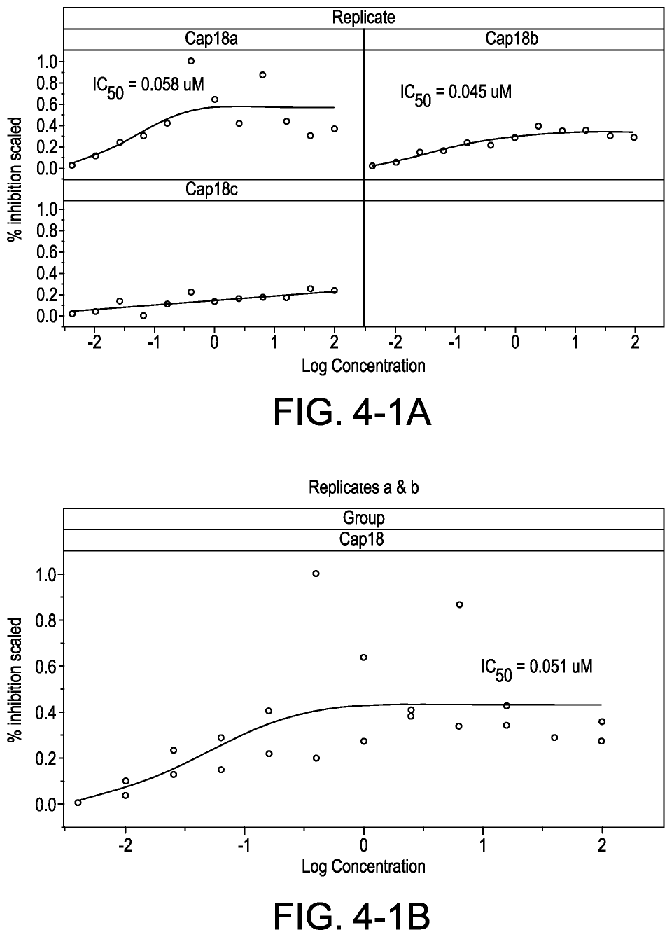

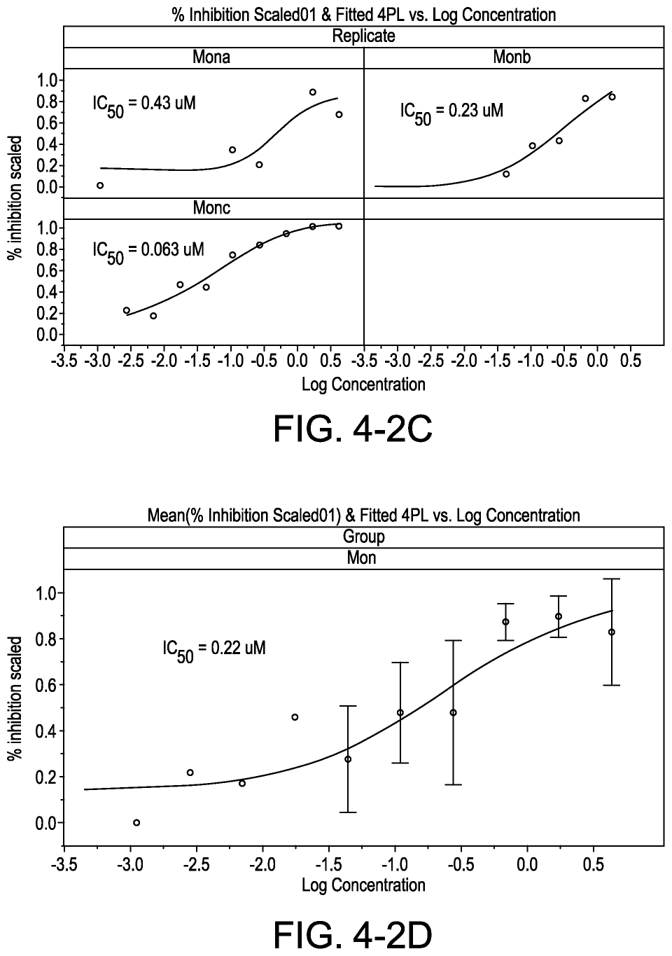

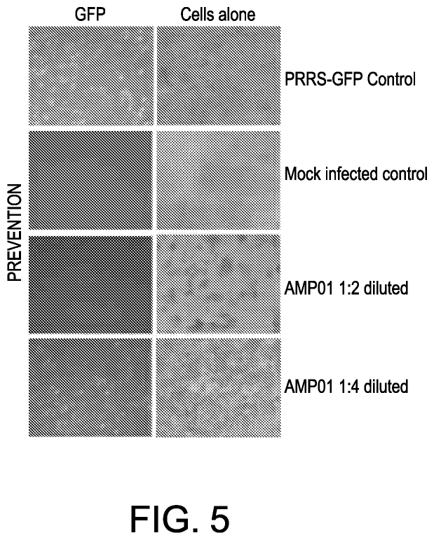

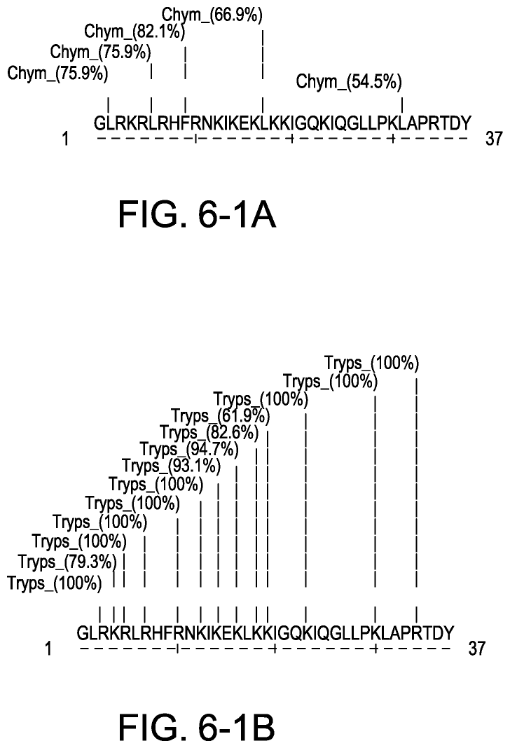

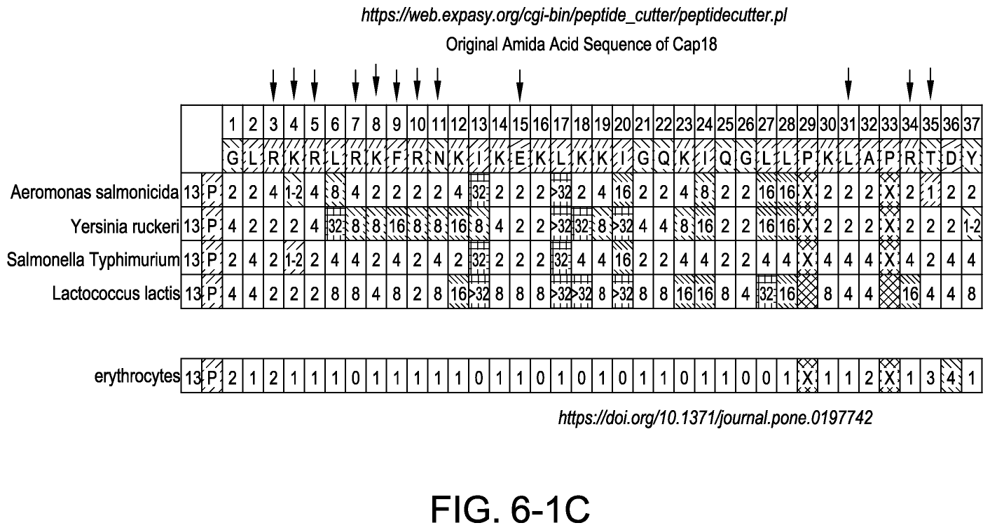

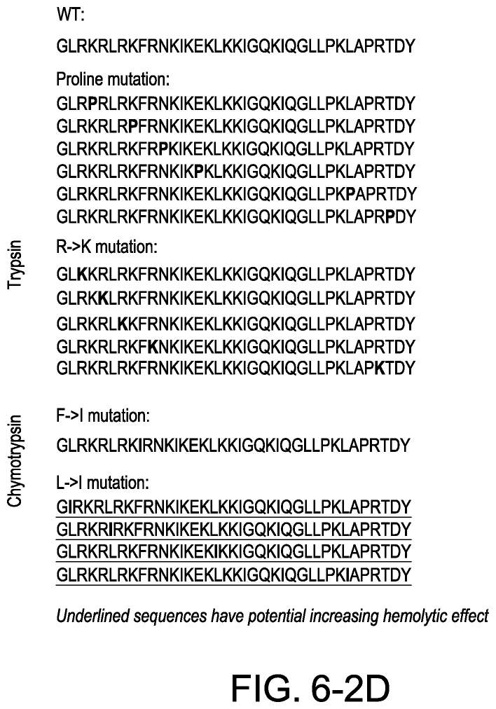

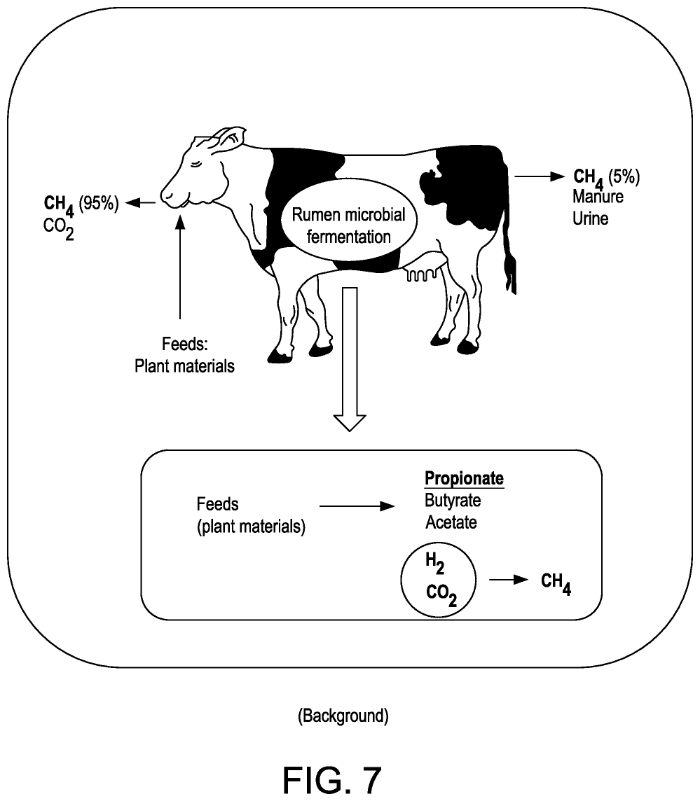

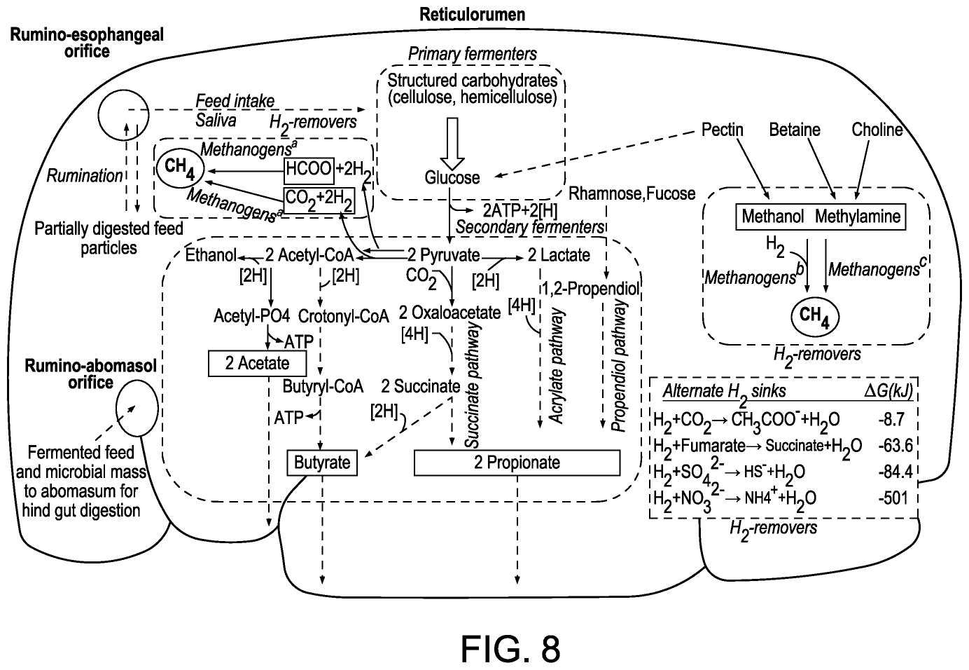

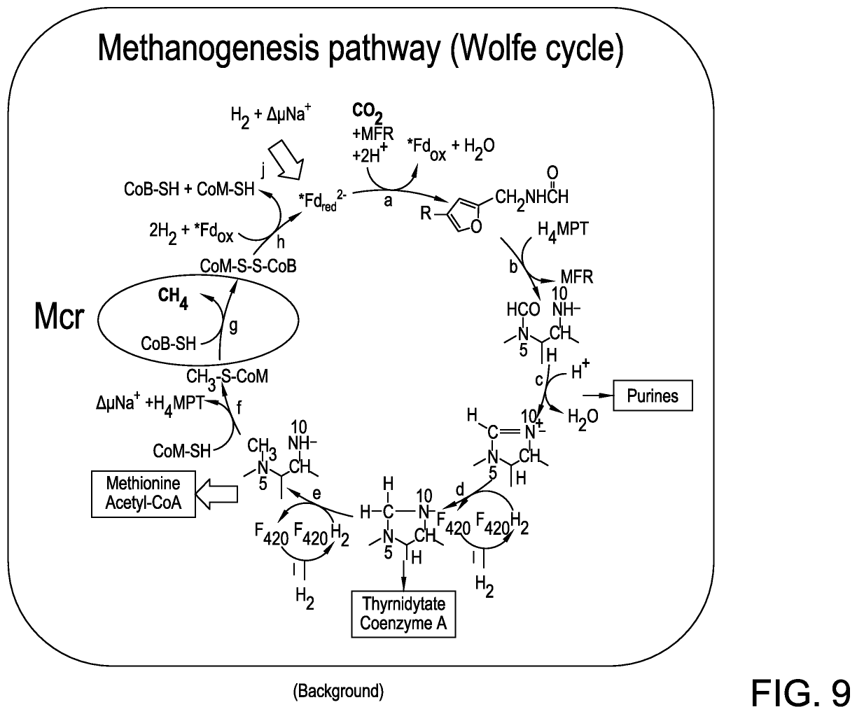

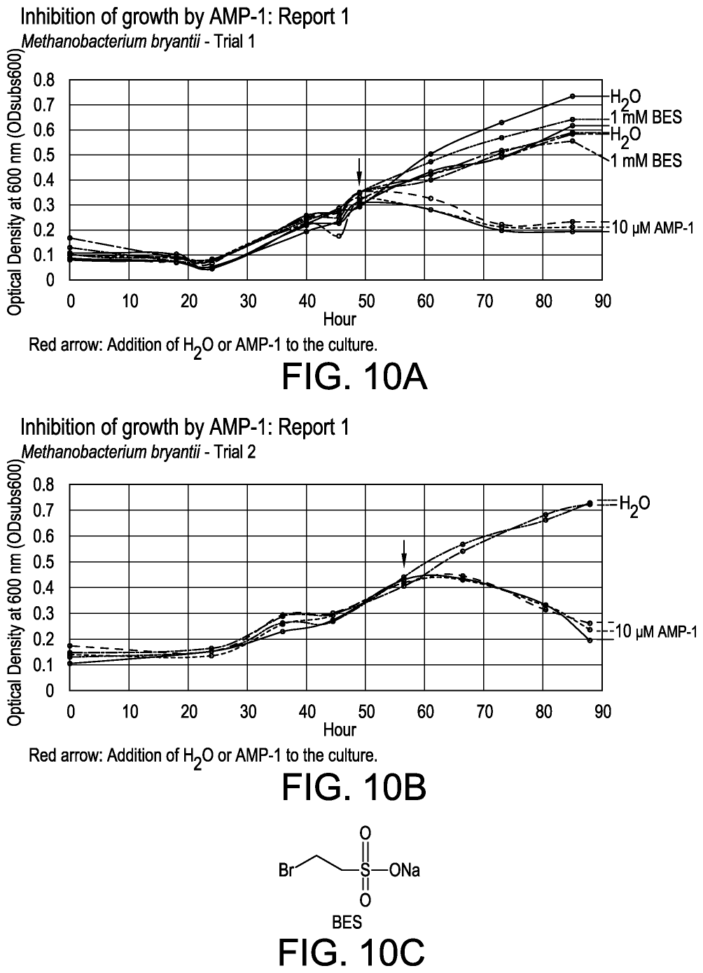

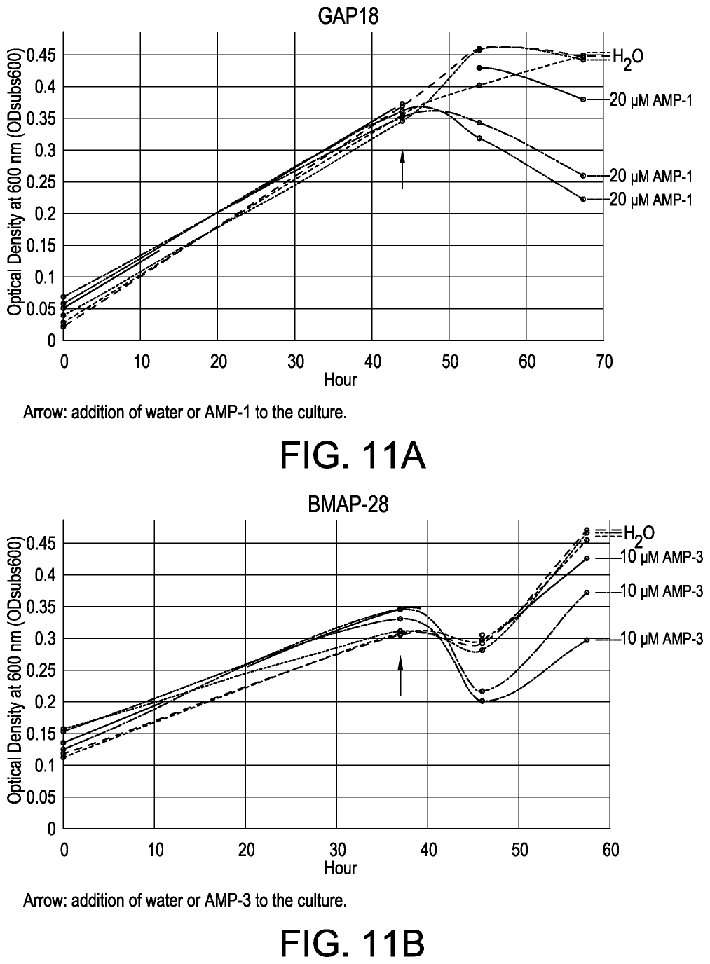

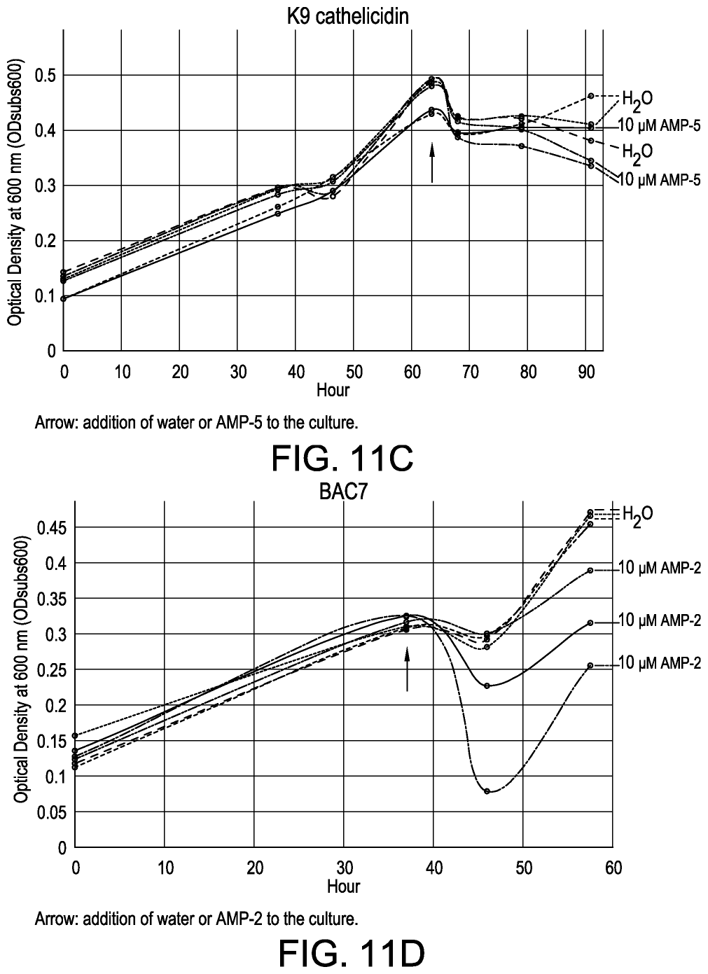

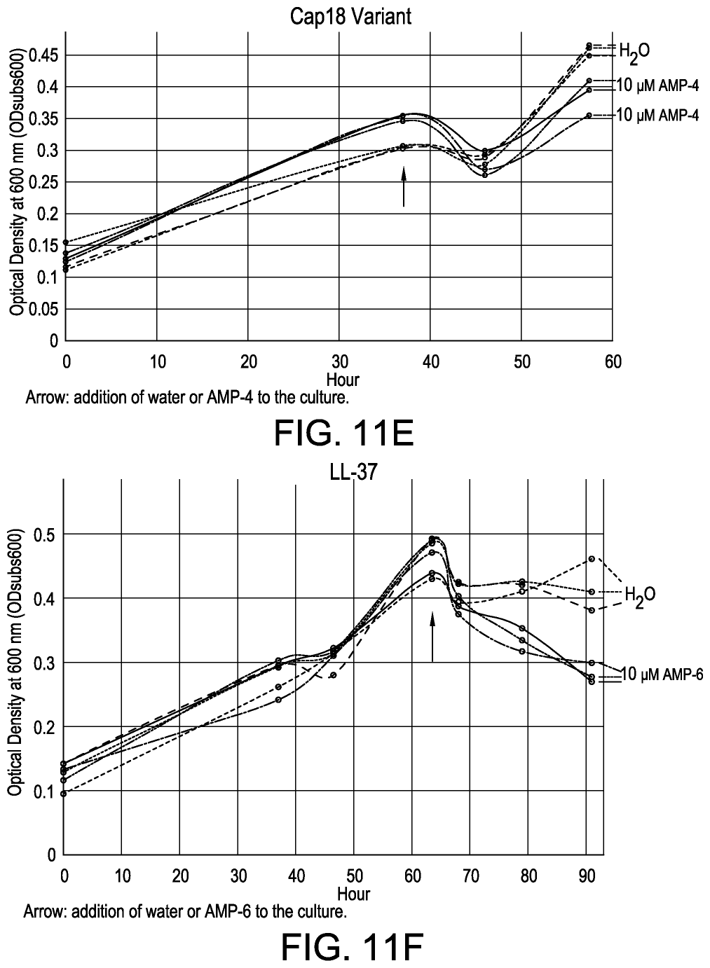

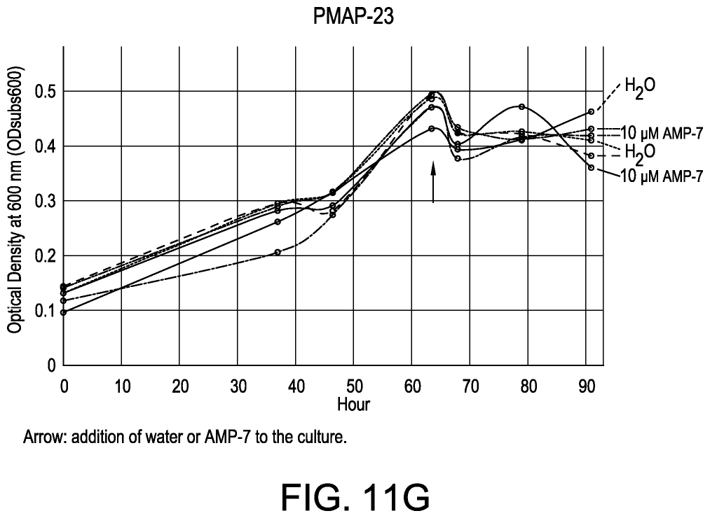

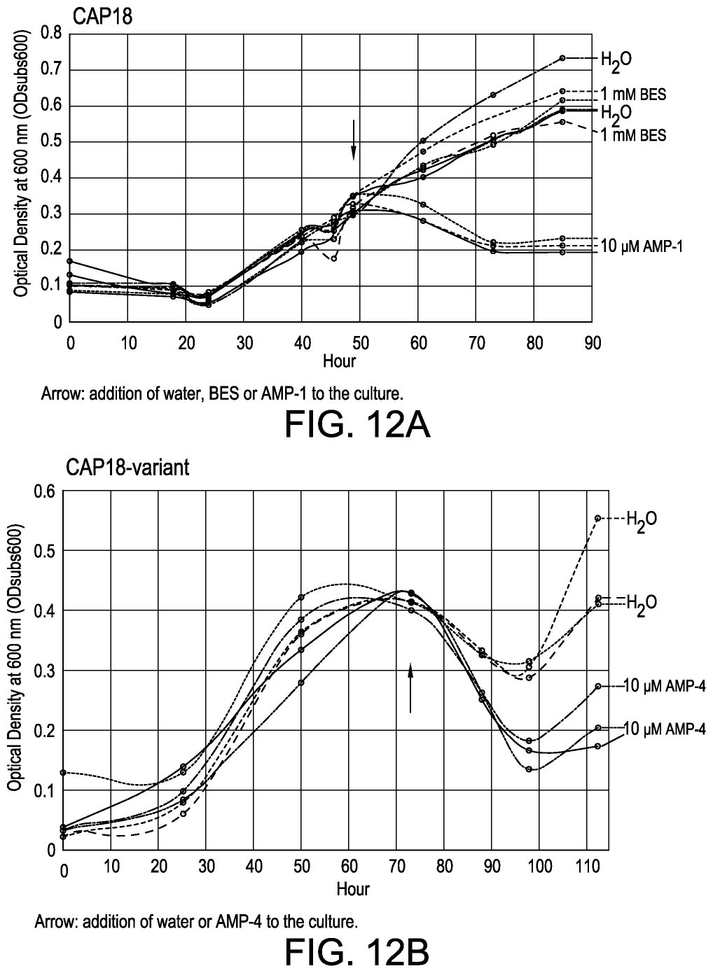

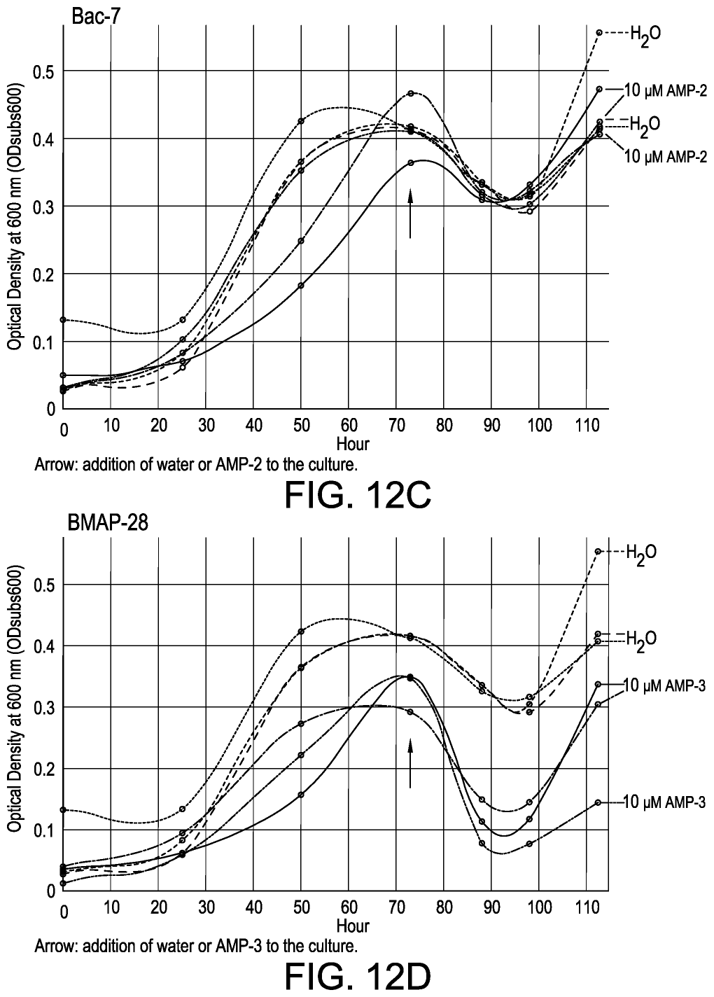

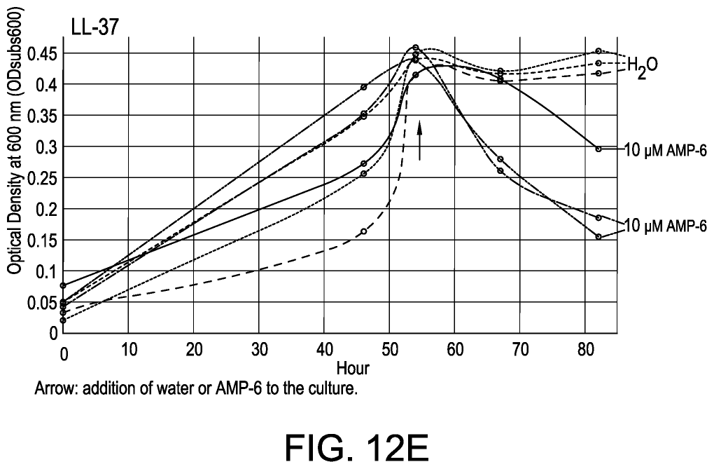

FIG. 2C shows AMP01 (CAP18) peptide vs control against virus, PRRSV (PRRS, swine) (FIG. 2C).

FIG. 3 depicts activity of CAP18 peptide (AMP01) against Giardia (Giardiosis, dogs). (A) After 1 d, the trophozoites in the control tube were fully alive (and overgrowing), as expected. (B) The trophozoites in the formonentin (FOR) positive control treated tube were still pear shaped, but with a grainy cytosol and most likely dead. (C) The trophozoites in the AMP01 treated tube were dead, shrunken and started to disintegrate. The sizes of the trophozoites can be compared as different sizes by comparing the white circles.

FIG. 4A-D depicts percent inhibition of Eimeria parasites by CAP18 peptides or monensin. Three independent experiments were conducted with CAP18 peptides—designated Cap18a, Cap18b and Cap18c. The CAP18 peptides were compared with monensin as a positive control. Three independent experiments with monensin (designated Mona, Monb and Monc).

FIG. 5 depicts inhibition of PRRS virus infection by AMP01 (wt CAP18) 1:2 diluted or 1:4 diluted versus mock infected control and PRRS-GFP control recognizing the virus.

FIG. 6A-D. Predicted chymotrypsin sites are depicted in A. Predicted trypsin sites are depicted in B. Various peptide amino acid mutations for consideration as reported are depicted in C. D provides peptides with proline mutation, R->K mutation, F->I mutation and L->I mutation.

FIG. 7 depicts methane emission by enteric fermentation.

FIG. 8. Methane production as by-product of ruminal fermentation and alternate H₂-sink pathways in the rumen. Primary and secondary microbial fermenters degrade structural carbohydrates into monomers and short chain fatty acids, respectively. The level of H₂ is kept low mostly by hydrogenotrophic methanogenic archaea and hydrogen-dependent methylotrophic methanogenic archaea (Methanogen^{a} and ^{b}, respectively). The accumulation of H2 inhibit reoxidation of NADH. When other electron acceptors are abundance (i.e., sulfate, nitrate, fumarate), electrons are channeled to these acceptors, shown in blue box “Alternate H₂ sinks”. Black dashed-lines, multi-step pathway; grey dashed-lines, host processes in rumen; black dotted-lines, absorption of volatile fatty acids by rumen wall.

FIG. 9 depicts the methanogenesis pathway (Wolfe cycle).

FIG. 10-A depicts inhibition of growth of Methanobacterium bryantii by wild type bacteria CAP 18, which is identified as AMP-1 in FIG. 10A, in a first in-vitro screening.

FIG. 10B depicts inhibition of growth of Methanobacterium bryantii by wild type bacteria CAP 18, which is identified as AMP-1 in FIG. 10B, in a second in-vitro screening.

FIG. 10-C depicts the structure of BES.

FIG. 11A-G. Anti-methanogenic activities of various antimicrobial peptides (CAP18, BMAP-28, K9 cathelicidin, BAC-, CAP18 variant, LL-37, PMAP-23, respectively) against rumen Methanogen, Methanobrevibacter ruminantium.

FIG. 12A-E. Anti-methanogenic activities of various antimicrobial peptides (CAP18, CAP18 variant, Bac-7, BMAP-28, LL-37, respectively) against rumen Methanogen, Methanobacter bryantii.

### DETAILED DESCRIPTION

### Definitions

In accordance with the present disclosure there may be employed conventional molecular biology, microbiology, and recombinant DNA techniques within the skill of the art. Such techniques are explained fully in the literature.

As used herein, “subject” includes humans and other mammals, including a human, or a non-human animal, and also birds and fish. A subject includes a bird, poultry, human or non-human animal. Specific examples of animals include bird, poultry, chickens, turkey, dogs, cats, cattle, horse, fish and swine. The chicken may be a broiler chicken, egg-laying or egg-producing chicken. As used herein, the term “poultry” includes domestic fowl, such as chickens, turkeys, ducks, quail, and geese.

The term “treating” or “treatment” of any disease or disorder refers, in one embodiment, to ameliorating the disease or disorder (i.e., arresting the disease or reducing the manifestation, extent or severity of at least one of the clinical symptoms thereof). In another embodiment ‘treating’ or ‘treatment’ refers to ameliorating at least one physical parameter, which may not be discernible by the subject. In yet another embodiment, ‘treating’ or ‘treatment’ refers to modulating the disease or disorder, either physically, (e.g., stabilization of a discernible symptom), physiologically, (e.g., stabilization of a physical parameter), or both. In a further embodiment, ‘treating’ or ‘treatment’ relates to slowing the progression of the disease. In an aspect, the term “alleviate” or “alleviation” refers to and includes the reduction in the manifestation, extent or severity of a disease or symptom(s) thereof, recognizing that such reduction can serve to reduce pain, suffering, physical or physiological deficit(s), and improve clinical parameters associated with a disease, while not curing or fully eliminating the disease.

The phrase “pharmaceutically acceptable” refers to molecular entities and compositions that are physiologically tolerable and do not typically produce an allergic or similar untoward reaction, such as gastric upset, dizziness and the like, when administered to a human or other animal.

The term “therapeutically effective amount” means that amount of a drug, compound, peptide, or pharmaceutical agent that will elicit the biological, physiological, clinical, or medical response of a subject that is being sought by a medical doctor or other clinician. The phrase “therapeutically effective amount” is used herein to include an amount sufficient to prevent, and preferably reduce by at least about 30 percent, more preferably by at least 50 percent, most preferably by at least 90 percent, a clinically significant change in the S phase activity of a target cellular mass, in the enlargement of an organ, in the accumulation of a substrate or protein, in a neurological deficit or impairment, or other feature of pathology such as for example, elevated blood pressure, fever or white cell count, enlargement of the spleen or liver as may attend its presence and activity.

The terms engineered “antimicrobial polypeptide,” “engineered polypeptide” and “antimicrobial polypeptide” are used interchangeably herein.

As used herein, the terms “treating”, “to treat”, or “treatment”, include restraining, slowing, stopping, reducing, ameliorating, or reversing the progression or severity of an existing symptom, disorder, condition, or disease. A treatment may be applied prophylactically or therapeutically.

The term “preventing” or “prevention” refers to a reduction in risk of acquiring or developing a disease or disorder (i.e., causing at least one of the clinical symptoms of the disease not to develop) in a subject that may be exposed to a disease-causing agent, or predisposed to the disease in advance of disease onset.

The term “prophylaxis” is related to and encompassed in the term “prevention”, and refers to a measure or procedure the purpose of which is to prevent, rather than to treat or cure a disease. Non-limiting examples of prophylactic measures may include the administration of vaccines; the administration of low molecular weight heparin to hospital patients at risk for thrombosis due, for example, to immobilization; and the administration of an anti-malarial agent such as chloroquine, in advance of a visit to a geographical region where malaria is endemic or the risk of contracting malaria is high.

The term “solvate” means a physical association of a compound useful in this disclosure with one or more solvent molecules. This physical association includes hydrogen bonding. In certain instances the solvate will be capable of isolation, for example when one or more solvent molecules are incorporated in the crystal lattice of the crystalline solid. “Solvate” encompasses both solution-phase and isolable solvates. Representative solvates include hydrates, ethanolates and methanolates.

A “replicon” is any genetic element (e.g., plasmid, chromosome, virus) that functions as an autonomous unit of DNA replication in vivo; i.e., capable of replication under its own control.

A “vector” is a replicon, such as plasmid, phage or cosmid, to which another DNA segment may be attached so as to bring about the replication of the attached segment.

A “DNA molecule” refers to the polymeric form of deoxyribonucleotides (adenine, guanine, thymine, or cytosine) in its either single stranded form, or a double-stranded helix. This term refers only to the primary and secondary structure of the molecule, and does not limit it to any particular tertiary forms. Thus, this term includes double-stranded DNA found, inter alia, in linear DNA molecules (e.g., restriction fragments), viruses, plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA).

An “origin of replication” refers to those DNA sequences that participate in DNA synthesis.

A DNA “coding sequence” is a double-stranded DNA sequence which is transcribed and translated into a polypeptide in vivo when placed under the control of appropriate regulatory sequences. The boundaries of the coding sequence are determined by a start codon at the 5′ (amino) terminus and a translation stop codon at the 3′ (carboxyl) terminus.

A coding sequence can include, but is not limited to, prokaryotic sequences, cDNA from eukaryotic mRNA, genomic DNA sequences from eukaryotic (e.g., mammalian) DNA, and even synthetic DNA sequences. A polyadenylation signal and transcription termination sequence will usually be located 3′ to the coding sequence.

Transcriptional and translational control sequences are DNA regulatory sequences, such as promoters, enhancers, polyadenylation signals, terminators, and the like, that provide for the expression of a coding sequence in a host cell.

A “promoter sequence” is a DNA regulatory region capable of binding RNA polymerase in a cell and initiating transcription of a downstream (3′ direction) coding sequence. For purposes of defining the present disclosure, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined by mapping with nuclease Si), as well as protein binding domains (consensus sequences) responsible for the binding of RNA polymerase. Eukaryotic promoters will often, but not always, contain “TATA” boxes and “CAT” boxes. Prokaryotic promoters contain Shine-Dalgarno sequences in addition to the −10 and −35 consensus sequences.

An “expression control sequence” is a DNA sequence that controls and regulates the transcription and translation of another DNA sequence. A coding sequence is “under the control” of transcriptional and translational control sequences in a cell when RNA polymerase transcribes the coding sequence into mRNA, which is then translated into the protein encoded by the coding sequence.

A “signal sequence” can be included before the coding sequence. This sequence encodes a signal peptide, N-terminal to the polypeptide, that communicates to the host cell to direct the polypeptide to the cell surface or secrete the polypeptide into the media, and this signal peptide is clipped off by the host cell before the protein leaves the cell. Signal sequences can be found associated with a variety of proteins native to prokaryotes and eukaryotes.

A “heterologous” region of a nucleic acid, RNA or DNA, construct is an Identifiable segment of RNA or DNA within a larger RNA or DNA molecule that is not found in association with the larger molecule in nature. Thus, when the heterologous region encodes a gene, the gene will usually be flanked by RNA or DNA that does not flank the genomic RNA or DNA in the genome of the source organism.

A “chimeric protein” or “fusion protein” comprises all or (preferably a biologically active) part of a first polypeptide operably linked to a heterologous polypeptide. Chimeric proteins or peptides are produced, for example, by combining two or more proteins having two or more active sites. In a chimeric or fusion protein, a first polypeptide may be covalently attached to an entity which may provide additional function or enhance the use or application of the first polypeptide(s), including for instance a tag, label, targeting moiety or ligand, a cell binding or cell recognizing motif or agent, an antibacterial agent, an antibody, an antibiotic. Exemplary labels include a radioactive label, such as the isotopes ³H, ¹⁴C, ³P, ³S, ³⁶Cl, ⁵¹Cr, ⁵⁷Co, ⁵⁸ Co, ⁵⁹Fe, ⁹⁰ Y, ¹²⁵I, ¹³¹I, and ¹⁸⁶Re. The label may be an enzyme, and detection of the labeled lysin polypeptide may be accomplished by any of the presently utilized or accepted colorimetric, spectrophotometric, fluorospectrophotometric, amperometric or gasometric techniques known in the art. Chimeric protein and peptides can act independently on the same or different molecules or targets, and hence have a potential to provide multiple activities, such as to treat or stimulate immune response against two or more different bacterial infections or Infective agents at the same time.

A chimeric protein or fusion protein includes wherein a first heterologous protein of interest is combined with another distinct protein or peptide of interest. A chimeric protein or fusion protein includes wherein a first heterologous protein of interest is combined with a targeting protein or targeting sequence which may direct the first heterologous protein to a particular cell type, a particular cell receptor, or a tissue or region of the body of an animal for instance. A chimeric protein or fusion protein includes wherein a first heterologous protein of interest is combined with a targeting protein or targeting sequence which may direct the first heterologous protein outside of the cell of expression, such as to be expressed or located systemically in an animal, or to the blood or local tissues in the animal. A chimeric protein includes wherein a first heterologous protein is combined with a label, tag or enzyme. A tag or label or enzyme may be a functional molecule. A tag or label may be an epitope. A tag or label may be a detectable molecule, protein or other entity. A tag or label may be a fluorescent molecule, a radioactive molecule, etc. Suitable fluorescent molecules are known and available in the art. A fluorescent molecule may be a green fluorescent protein (GFP) for example.

The term “bacteriocidal” refers to being capable of killing bacterial cells.

The term “bacteriostatic” refers to capable of inhibiting bacterial growth, including Inhibiting growing bacterial cells.

A wide range of antimicrobial peptides is secreted in plants and animals to challenge attack by foreign viruses, bacteria or fungi (Boman, H. G. (2003) J. Intern. Med. 254 (3):197-215). These form part of the innate immune response to infection, which is short term and fast acting relative to humoral immunity. These peptides are heterogeneous in length, sequence and structure, but most are small, cationic and amphipathic (Zasloff, M. (2002) Nature 415(6870):389-395). Antimicrobial peptides have been considered as prospective antibiotics agents because their effect is rapid, broad spectrum and indifferent to resistance to standard antibiotics such as penicillins (Fischetti, V. A. (2003) Ann. N. Y. Acad. Sci. 987:207-214; Hancock, R. E. (1999) Drugs 57(4):469-473). Various antimicrobial peptides have been studied in order to understand the relationship between the structural features of the peptides and their antimicrobial activity, for the purpose of designing a new generation of antibiotics. While the external cell wall may be the initial target, evidence suggests that antimicrobial peptides act by lysing bacterial membranes. Cells become permeable following exposure to peptides, and their membrane potential is correspondingly reduced. While the actual target and mode of action of antimicrobial peptides are incompletely understood, proposed models emphasize the need to coat or cover a significant part of the membrane in order to produce a lethal effect.

Protamines or polycationic amino acid peptides containing combinations of one or more recurring units of cationic amino acids, such as arginine (R), tryptophan (W), lysine (K), even synthetic polyarginine, polytryptophan, polylysine, have been shown to be capable of killing microbial cells. These peptides cross the plasma membrane to facilitate uptake of various biopolymers or small molecules (Mitchell D J et al (2002) J Peptide Res 56(5):318-325).

In contrast to antibiotics, pathogens are unlikely to develop resistance to antimicrobial peptides, including CAP18 peptides and variant CAP18 peptides of the disclosure, due to their rapid action on bacterial membrane. Lytic peptides have been evaluated for antibiotic resistance and shown not to lead to resistance. This is confirmed by treating susceptible or target bacteria, such as M. haemolytica and P. multocida, in vitro with different concentrations of one or more of the CAP18 peptides, including the variant CAP 18 peptides of the disclosure, and observing and evaluating for potential resistant mutants.

The success of antimicrobial peptides thus far has been limited, largely due to the requirement that they be present in a fairly high concentration to achieve killing. This high concentration can exert a potentially cytotoxic effect on human erythrocytes as well as other cells and tissues for example. The high concentrations are due, in part to the susceptibility of antimicrobial peptides to native proteases in an animal or otherwise produced and present at the site of therapeutic target.

A particularly useful anti-microbial peptide is wild type CAP-18. Variant CAP18 peptides that retain pathogen inhibition or killing, including bacterial, viral, and parasitic inhibition or killing, and which have enhanced protease resistance, improved temperature resistance, and/or improved stability are provided. In an embodiment, variant CAP18 peptides are provided that have one or more amino acid substitutions or variations compared to wild type CAP18 sequence. In an embodiment, variant CAP18 peptides are provided having two or more amino acid substitutions or variations compared to wild type CAP18 sequence. In an embodiment, variant CAP18 peptides are provided having at least two amino acid substitutions or variations compared to wild type CAP18 sequence. In an embodiment, variant CAP18 peptides are provided having three or more amino acid substitutions or variations compared to wild type CAP18 sequence. In a particular embodiment wild type CAP18 sequence (rabbit) is set out as shown below:

(SEQ ID NO: 1)
GLRKRLRKFRNKIKEKLKKIGQKIQGLLPKLAPRTDY.

Human wild type CAP18 peptide Is as follows: LLGDFFRKSKEKIGKEFKRIVQRIKDFLRNLVPRTES (SEQ ID NO: 118).

CAP18 has antimicrobial activity against a variety of bacterial pathogens, including Staphylococcus aureus, Streptococcus pneumonia, Escherichia coli, Pseudomonas aeruginosa, Salmonella Typhimurium, Yersinia ruckeri, Aeromonas salmonicida, Campylobacter jejuni, Enterococcus faecalis, and Listeria monocytogenes. In addition to antimicrobial activity, CAP18 potently binds LPS and has scavenges LPS to reduce inflammation. Thus, CAP18 not only has the potential to address multiple microbial etiologies in animal/human health, it can also serve as an LPS scavenger to reduce the potential for LPS-induced Inflammatory responses. CAP18 peptide (wild type peptide also herein designated as AMP01) has application and utility for multiple indications in livestock, poultry and aquatic species. CAP18 demonstrates anti-bacterial and killing activity against various bacteria relevant to diseases in animals, particularly, Mannheimia and Pasteurella (BRD, cattle), E. coli (Colibacillosis, poultry), Salmonella (Salmonellosis, poultry), C. jejuni (Campylobacteriosis, poultry), P. salmonis (SRS, salmon). CAP18 peptide is also effective in inhibiting activity of parasites of significance in animals such as Giardia (Giardiosis, dogs) and Eimeria (Coccidiosis, poultry).

In an embodiment an engineered polypeptide variant of CAP18 includes:

(SEQ ID NO: 103)
X₁X₂X₃KX₄X₅X₆KX₇X₈NKIKEKLKKIGQKIQGLLP
KLAPX₉TDX₁₀,

wherein X1 Is G or CWTKSIPPKPC-G (SEQ ID NO: 104),
X2 is C or L,
X3 is R or K,
X4 is P, A, V, I, L, M, F, Y, or W,
X5 Is C or L,
X6 Is R or K,
X7 is I or K,
X8 is R, I, or K,
X9 is R or K, and
X10 Is Y or Y-CWTKSIPPKPC (SEQ ID NO: 105),
wherein the polypeptide does not include GLRKRLRKFRNKIKEKLKKIGQKIQGLLPKLAPRTDY (SEQ ID NO: 1).

In an embodiment, the engineered polypeptide includes one of the following variants of CAP18:

SEQ ID NO: 13)
GLRKRLRKIRNKIKEKLKKIGQKIQGLLPKLAPRTDY
(SEQ ID NO: 49)
GLRKRLRKIRNKIKEKLKKIGQKIQGLLPKLAPRTDYCWTKSIPPKPC;
(SEQ ID ID NO: 46)
CWTKSIPPKPCGLRKRLRKIRNKIKEKLKKIGQKIQGLLPKLAPRTDY;
(SEQ ID NO: 47)
CWTKSIPPKPCGLRKRLRKIRNKIKEKLKKIGQKIQGLLPKLAPRTDY
CWTKSIPPKPC;
(SEQ ID NO: 18)
CWTKSIPPKPCGLRKRLKKIKNKIKEKLKKIGQKIQGLLPKLAPRTDY;
(SEQ ID NO: 19)
CWTKSIPPKPCGLRKILKKIKNKIKEKLKKIGQKIQGLLPKLAPRTDY;
(SEQ ID NO: 20)
CWTKSIPPKPCGLRKKLKKIRNKIKEKLKKIGQKIQGLLPKLAPRTDY;
(SEQ ID NO: 21)
CWTKSIPPKPCGLRKRLRKIKNKIKEKLKKIGQKIQGLLPKLAPKTDY;
(SEQ ID NO: 22)
CWTKSIPPKPCGCRKPLRKIRNKIKEKLKKIGQKIQGLLPKLAPRTDY;
(SEQ ID NO: 23)
CWTKSIPPKPCGCRKPCRKIRNKIKEKLKKIGQKIQGLLPKLAPRTDY;
(SEQ ID NO: 30)
CWTKSIPPKPCGLRKRLRKIRNKIKEKLKKIGQKIQGLLPKLAPRTDY;
(SEQ ID NO: 31)
GLRKRLKKIKNKIKEKLKKIGQKIQGLLPKLAPRTDY;
(SEQ ID NO: 32)
GLRKILKKIKNKIKEKLKKIGQKIQGLLPKLAPRTDY;
(SEQ ID NO: 33)
GLRKKLKKIRNKIKEKLKKIGQKIQGLLPKLAPRTDY;
(SEQ ID NO: 34)
GLRKRLRKIKNKIKEKLKKIGQKIQGLLPKLAPKTDY;
(SEQ ID NO: 35)
GCRKPLRKIRNKIKEKLKKIGQKIQGLLPKLAPRTDY;
(SEQ ID NO: 36)
GCRKPCRKIRNKIKEKLKKIGQKIQGLLPKLAPRTDY;
(SEQ ID NO: 37)
GLRKKLKKIKNKIKEKLKKIGQKIQGLLPKLAPRTDY;
(SEQ ID NO: 38)
GLKKKLKKIKNKIKEKLKKIGQKIQGLLPKLAPRTDY;
(SEQ ID NO: 39)
GLKKKLKKIKNKIKEKLKKIGQKIQGLLPKLAPKTDY;
(SEQ ID NO: 40)
GLRKILRKIRNKIKEKLKKIGQKIQGLLPKLAPRTDY;
(SEQ ID NO: 41)
GLKKILKKIKNKIKEKLKKIGQKIQGLLPKLAPRTDY;or
(SEQ ID NO: 42)
GLKKKLRKIRNKIKEKLKKIGQKIQGLLPKLAPRTDY.

In embodiments, the compositions and methods of the present disclosure include variants of one or more polypeptides set forth in Table 2, including CAP18 variants, BMAP28 variants (CATHL5; bovine), Bac7 variants (CATHL3; bovine rumen), k9Cath variants (canine) and PMAP36 variants (porcine).

TABLE 2

| Antimicrobial | | |
|---|---|---|
| peptide | Source | Size (aa) |
| BMAP-28 (CATHL5) | Bovine | 28 |
| Bac7 (CATHL3) | Bovine | 60 |
| | (rumen) | |
| PMAP23 | Porcine | 23 |
| PMAP36 | Porcine | 36 |
| k9Cath | Canine | 38 |
| LL-37 | Human | 37 |
| cap18_R2Kv3_NTinh | Human | 48 |
| NK-2 | Porcine | 27 |

The wild type sequences for each of the antimicrobial peptides of Table 2 are set forth below.

>BMAP-28 (CATHL5)-Bovine (28 aa)
(SEQ ID NO: 50)
GGLRSLGRKILRAWKKYGPIIVPIIRIG
>Bac7 (CATHL3)-bovine (rumen) (60 aa)
(SEQ ID NO: 63)
RRIRPRPPRLPRPRPRPLPFPRPGPRPIPRPL
PFPRPGPRPIPRPLPFPRPGPRPIPRPL;
> PMAP23-swine (23 aa)
(SEQ ID NO: 119)
RIIDLLWRVRRPQKPKFVTVWVR
> PMAP36-swine (36 aa)
(SEQ ID NO: 90)
GRFRRLRKKTRKRLKKIGKVLKWIPPIVGSIPLGCG;
> k9Cath-canine (38 aa)
(SEQ ID NO: 77)
RLKELITTGGQKIGEKIRRIGQRIKDFFKNLQPREEKS;
>LL-37-human (37 aa)
(SEQ ID NO: 118)
LLGDFFRKSKEKIGKEFKRIVQRIKDFLRNLVPRTES;
>cap18_R2Kv3_NTinh-rabbit (48 aa)
(SEQ ID NO: 18)
CWTKSIPPKPCGLRKRLKKIKNKIKEKLKKIGQKIQG
LLPKLAPRTDY;
>NK-2-porcine (27 aa)
(SEQ ID NO: 120)
KILRGVCKKIMRTFLRRISKDILTGKK.

Another AMP which can be used in the compositions and methods described herein is Nisin. The sequence for nisin (from Lactobacillus) is as follows:

(SEQ ID NO: 121)
MSTKDFNLDLVSVSKKDSGASPRITSISL
CTPGCKTGALMGCNMKTATCHCSIHVSK

The other antimicrobial cathelicidin polypeptides based on BMAP28 (CATHL5; bovine), Bac7 (CATHL3; bovine rumen), k9Cath (canine) and PMAP36 (porcine) are provided as follows in which alternative N terminal or C terminal tags or additional sequence are shown in bold with a comparison of variant amino acid sequence with wild type sequence being shown in bold and underlined.

In an embodiment, the present disclosure provides an engineered polypeptide variant of BMAP28, the polypeptide including:

(SEQ ID NO: 107)
X₁GX₂X₃SLGX₄KXSLX₆AX₇KKX₈GPX₉IVPIIX₁₀IG,

wherein X1 Is G, CWTKSIPPKPC-G (SEQ ID NO: 104) or CRKP-G (SEQ ID NO: 108),
X2 is L or A,
X3 is R or K,
X4 is R or K,
X5 is I or A,
X6 is R or K,
X7 is W, I or A,
X8 is Y, I or A,
X9 is I or A, and
X10 is R or K,
wherein the polypeptide does not include GGLRSLGRKILRAWKKYGPIIVPIIRIG (SEQ ID NO: 50).

In an embodiment, the engineered polypeptide includes one of the following variants of BMAP28:

(SEQ ID NO: 51)
CWTKSIPPKPCGGLRSLGRKILRAWKKYGPIIVPIIRIG;
(SEQ ID NO: 53)
CRKPGGLRSLGRKILRAWKKYGPIIVPIIRIG;
(SEQ ID NO: 55)
GGLKSLGKKILRAWKKYGPIIVPIIRIG;
(SEQ ID NO: 56)
GGLRSLGKKILKAWKKYGPIIVPIIRIG;
(SEQ ID NO: 57)
GGLRSLGRKILKAWKKYGPIIVPIIKIG;
(SEQ ID NO: 58)
GGLKSLGKKILKAWKKYGPIIVPIIKIG;
(SEQ ID NO: 59)
GGLRSLGRKILRAIKKYGPIIVPIIRIG;
(SEQ ID NO: 60)
GGLRSLGRKILRAWKKIGPIIVPIIRIG;
(SEQ ID NO: 61)
GGLRSLGRKILRAIKKIGPIIVPIIRIG;
or
(SEQ ID NO: 62)
GGARSLGRKALRAAKKAGPAIVPIIRIG.

In an embodiment, the present disclosure provides an engineered polypeptide, truncated BMAP28, the polypeptide including:

(SEQ ID NO: 109)
XGLRSLGRKILRAWKKYG,

wherein X is G, CWTKSIPPKPC-G (SEQ ID NO: 104), or CRKP-G (SEQ ID NO: 108).

In an embodiment, the present disclosure provides an engineered polypeptide variant of BAC7, the polypeptide including:

(SEQ ID NO: 110)
X₁X₂IRPRPPX₃LPRPRPRPLPX₄PRPGPRPIPR
PLPX₅PRPGPRPIPRPLPX₆PRPGPRPIPRPL,

wherein X1 is R, CWTKSIPPKPC-R (SEQ ID NO: 111), CRKP-R (SEQ ID NO: 112) or K,
X2 is R or K,
X3 is R or K,
X4 is F or I,
X5 is F or I, and
X6 is F or I,
wherein the polypeptide does not include

(SEQ ID NO: 63)
RRIRPRPPRLPRPRPRPLPFPRPGPRPIPRPLPFPRPGPRPIPRPLPFP
RPGPRPIPRPL.

In an embodiment, the engineered polypeptide variant of BAC7 includes one of the following:

(SEQ ID NO: 64)
CWTKSIPPKPCRRIRPRPPRLPRPRPRPLPFPRPGPRPIPRPLPFPRPG
PRPIPRPLPFPRPGPRPIPRPL;
(SEQ ID NO: 65)
CRKPRRIRPRPPRLPRPRPRPLPFPRPGPRPIPRPLPFPRPGPRPIPRP
LPFPRPGPRPIPRPL;
(SEQ ID NO: 70)
RRIRPRPPRLPRPRPRPLPIPRPGPRPIPRPLPIPRPGPRPIPRPLPFP
RPGPRPIPRPL;
(SEQ ID NO: 71)
RRIRPRPPRLPRPRPRPLPFPRPGPRPIPRPLPIPRPGPRPIPRPLPIP
RPGPRPIPRPL;
(SEQ ID NO: 72)
RRIRPRPPRLPRPRPRPLPIPRPGPRPIPRPLPIPRPGPRPIPRPLPIP
RPGPRPIPRPL;
(SEQ ID NO: 73)
KKIRPRPPRLPRPRPRPLPFPRPGPRPIPRPLPFPRPGPRPIPRPLPFP
RPGPRPIPRPL;
(SEQ ID NO: 74)
RKIRPRPPKLPRPRPRPLPFPRPGPRPIPRPLPFPRPGPRPIPRPLPFP
RPGPRPIPRPL;
or
(SEQ ID NO: 75)
KKIRPRPPKLPRPRPRPLPFPRPGPRPIPRPLPFPRPGPRPIPRPLPFP
RPGPRPIPRPL.

In an embodiment, the present disclosure provides an engineered polypeptide, truncated BAC7, the polypeptide including:

(SEQ ID NO: 113)
X₁X₂IRPRPPX₃LPRPRPR,

wherein X1 is R, CWTKSIPPKPC-R (SEQ ID NO: 111) or CRKP-R (SEQ ID NO: 112),
X2 is R or K, and
X3 is R or K.

In an embodiment, the engineered polypeptide, truncated BAC7 includes one of the following:

(SEQ ID NO: 66)
RRIRPRPPRLPRPRPR;
(SEQ ID NO: 67)
CWTKSIPPKPCRRIRPRPPRLPRPRPR;
(SEQ ID NO: 68)
CRKPRRIRPRPPRLPRPRPR;
or
(SEQ ID NO: 76)
KKIRPRPPKLPRPRPR.

In an embodiment, the present disclosure provides an engineered polypeptide variant of K9CATH, the polypeptide including:

(SEQ ID NO: 114)
X₁LKELITTGGQKIGEKIX₂X₃IGQRIKDX₄X₅KNLQPX₆EEKS,

wherein X1 is R, CWTKSIPPKPC-R (SEQ ID NO: 111), CRKP-R (SEQ ID NO: 112) or K,
X2 is R or K,
X3 is R or K,
X4 is F or I,
X5 is F or I, and
X6 is R or K,
wherein the polypeptide does not include

(SEQ ID NO: 77)
RLKELITTGGQKIGEKIRRIGQRIKDFFKNLQPREEKS.

In an embodiment, the engineered polypeptide of K9CATH includes one of the following:

(SEQ ID NO: 83)
RLKELITTGGQKIGEKIRRIGQRIKDIIKNLQPREEKS;
(SEQ ID NO: 84)
KLKELITTGGQKIGEKIKRIGQRIKDFFKNLQPREEKS;
(SEQ ID NO: 85)
RLKELITTGGQKIGEKIKKIGQRIKDFFKNLQPREEKS;
(SEQ ID NO: 86)
RLKELITTGGQKIGEKIRKIGQRIKDFFKNLQPKEEKS;
or
(SEQ ID NO: 87)
RLKELITTGGQKIGEKIKKIGQRIKDFFKNLQPKEEKS.

In an embodiment, the present disclosure provides an engineered polypeptide, truncated K9CATH, the polypeptide including:

(SEQ ID NO: 115)
X₁LKELITTGGQKIGEKIX₂X₃IG,

wherein X1 is R, CWTKSIPPKPC-R (SEQ ID NO: 111) or CRKP-R (SEQ ID NO: 112),
X2 is R or K, and
X3 is R or K.

In an embodiment, the engineered polypeptide, truncated K9CATH includes one of the following:

(SEQ ID NO: 88)
RLKELITTGGQKIGEKIKKIG;
or
(SEQ ID NO: 89)
CWTKSIPPKPCRLKELITTGGQKIGEKIKKIG.

In an embodiment, the present disclosure provides an engineered polypeptide, truncated PMAP36, the polypeptide including:

(SEQ ID NO: 116)
X₁X₂X₃RX₄LRKX₅TRX₆X₇LKX₈IGKVLKX₉I,

wherein X1 is G, CWTKSIPPKPC-G (SEQ ID NO: 104) or CRKP-G (SEQ ID NO: 108),
X2 is R or V,
X3 is for L,
X4 is R or V,
X5 is K or V,
X6 is K or V,
X7 Is R or V,
X8 is K or V, and
X9 is W or L

In an embodiment, the engineered polypeptide, truncated PMAP36 Includes one of the following:

(SEQ ID NO: 96)
GRFRRLRKKTRKRLKKIGKVLKLI;
(SEQ ID NO: 97)
GRLRRLRKKTRKRLKKIGKVLKLI;
(SEQ ID NO: 98)
GVFRVLRKVTRVVLKVIGKVLKLI;
or
(SEQ ID NO: 99)
GVLRVLRKVTRVVLKVIGKVLKLI.

In an embodiment, the present disclosure provides an engineered polypeptide, truncated 2 of PMAP36, the polypeptide including:

(SEQ ID NO: 117)
X₁X₂LRKKTRKRLKKIGKVLKX₃I,

wherein X1 is R or CWTKSIPPKPC-R (SEQ ID NO: 111),
X2 is R or K, and
X3 is W or L.

The present disclosure also provides a solution to the problem of greenhouse gas emissions and, specifically, to the problem of methane emission from livestock, such as ruminants, thereby improving livestock production sustainability and ruminal feed efficiency. Specifically, the present disclosure provides for the development of compositions for reduction of enteric methane gas emissions from livestock, particularly ruminants, to reduce the carbon footprint from livestock production, to provide for manure management and to provide for ruminal feed efficiency.

Sustainability of livestock ruminant production and Improvement of feed efficiency are thus advantages of the present disclosure. In particular, reduced methane leads to increased feed efficiency which leads to sustainable livestock production. A direct effect on reduction of enteric methane emission (a forecasted 30-50% reduction) is contemplated.

The present disclosure provides a mitigation strategy for enteric methane production. Specifically, the present disclosure provides for the use of anti-methanogenic compounds and, more particularly, to the use of anti-microbial peptides (AMPs) to mitigate enteric methane productions in animals, such as livestock and, more particularly, ruminants. Useful anti-microbial peptides include cathelicidin AMPs from various hosts. As anti-methanogenic compounds, AMPs have a direct effect on methanogens. The target of the AMPs is methanogen's cell membrane. Without wishing to be bound by any particular theory, the AMPs compromise cell membrane integrity.

AMPs, which are post-biotics, advantageously provide a potent methanogenic killing effect. Moreover, by using AMPs, it is possible to achieve broad selectivity against microbes. AMPs can have a beneficial effect on the rumen microbiome (enriching H₂ consumers). By in-vitro screening of the engineered antimicrobial peptides of this disclosure, it is possible to identify antimicrobial peptides that inhibit the growth of methanogen strains such as at least one of Methanobrevibacter ruminantium DSM 1093, Methanosphaera stadtmanae DSM 3091, Methanomicrobium mobile DSM 1539, Methanobacterium bryantii, Methanobrevibacter gottchackii, Methanobrevibacter olleyae, Methanobrevibacter thauerii, Methanomassilicoccus luminyensis or Methanosarcina barkeri, and combinations thereof.

In embodiments of the disclosure the CAP18 variant peptides, including one or more variant CAP18 peptide, are applicable to the reduction of greenhouse gas emissions, particularly methane emissions, from an animal such as livestock and, more particularly, rumen. Methods of killing bacteria in an animal or inhibiting colonization of an animal by bacteria are included in the disclosure.

The present disclosure provides AMPS that target the cell membrane of methanogens in the gastrointestinal tract of an animal. Methanogens which can be targets of the probiotics of the present disclosure include, without limitation, at least one of Methanobrevibacter ruminantium DSM 1093, Methanosphaera stadtmanae DSM 3091, Methanomicrobium mobile DSM 1539, Methanobacterium bryantii, Methanobrevibacter gottchackii, Methanobrevibacter olleyae, Methanobrevibacter thauerii, Methanomassilicoccus luminyensis or Methanosarcina barkeri, and combinations thereof.

The compositions and methods described herein may provide the AMPs described herein by post-biotic or vectored delivery.

In some aspects, the compositions described above are used to reduce bacterial load, particularly pathogenic bacteria or clinically significant bacteria, including the number or amount of bacteria in the gut or gastrointestinal tract of an animal. The bacteria or archaea may be selected from at least one of Methanobrevibacter ruminantium DSM 1093, Methanosphaera stadtmanae DSM 3091, Methanomicrobium mobile DSM 1539, Methanobacterium bryantii, Methanobrevibacter gottchackii, Methanobrevibacter olleyae, Methanobrevibacter thauerii, Methanomassilicoccus luminyensis or Methanosarcina barkeri, and combinations thereof.

In some aspects, the compositions described above are used to reduce transmission of bacteria, particularly pathogenic bacteria, in an animal pen or in a group or herd of animals. In some aspects, the compositions described above are used to reduce transmission in an animal pen or in a group or herd of animals of at least one of Methanobrevibacter ruminantium DSM 1093, Methanosphaera stadtmanae DSM 3091, Methanomicrobium mobile DSM 1539, Methanobacterium bryantii, Methanobrevibacter gottchackii, Methanobrevibacter olleyae, Methanobrevibacter thauerii, Methanomassilicoccus luminyensis or Methanosarcina barkeri, and combinations thereof.

In embodiments of the disclosure, an animal may include a farmed animal or livestock or a domesticated animal. Livestock or farmed animal may include cattle (e.g., cows or bulls (including calves)), poultry (including broilers, chickens and turkeys), pigs (including piglets), birds, aquatic animals such as fish, agastric fish, gastric fish, freshwater fish such as salmon, cod, trout and carp, e.g. koi carp, marine fish such as sea bass, and crustaceans such as shrimps, mussels and scallops), horses (including race horses), sheep (including lambs). As used herein, the term “ruminants” includes, without limitation, extensive beef cattle, intensive beef cattle and dairy cattle.

The compositions may further include one or more component or additive. The one or more component or additive may be a component or additive to facilitate administration, for example by way of a stabilizer or vehicle, or by way of an additive to enable administration to an animal such as by any suitable administrative means, including in aerosol or spray form, in water, in feed or in an injectable form. Administration to an animal may be by any known or standard technique. These include oral ingestion, gastric Intubation, or broncho-nasal spraying. The compositions disclosed herein may be administered by immersion, intranasal, intramammary, topical, mucosally, or inhalation.

In some embodiments, the composition does not include antibiotics. Exemplary antibiotics include tetracycline, bacitracin, tylosin, salinomycin, virginiamycin and bambermycin.

The compositions described above may include a carrier suitable for animal consumption or use. Examples of suitable carriers include edible food grade material, mineral mixture, gelatin, cellulose, carbohydrate, starch, glycerin, water, glycol, molasses, corn oil, animal feed, such as cereals (barley, maize, oats, and the like), starches (tapioca and the like), oilseed cakes, and vegetable wastes. In some embodiments, the compositions include vitamins, minerals, trace elements, emulsifiers, aromatizing products, binders, colorants, odorants, thickening agents, and the like.

In some embodiments, the compositions include one or more biologically active molecule or therapeutic molecule. Examples of the aforementioned include ionophore; vaccine; antibiotic; antihelmintic; virucide; nematicide; amino acids such as methionine, glycine, and arginine; fish oil; krill oil; and enzymes.

In some embodiments, the compositions or combinations may additionally include one or more prebiotic. In some embodiments, the compositions may be administered along with or may be coadministered with one or more prebiotic. Prebiotics may include organic acids or non-digestible feed ingredients that are fermented in the lower gut and may serve to select for beneficial bacteria. Prebiotics may include mannan-oligosaccharides, fructo-oligosaccharides, galacto-oligosaccharides, chito-oligosaccharides, isomalto-oligosaccharides, pectic-oligosaccharides, xylo-oligosaccharides, and lactose-oligosaccharides.

The composition may be formulated as animal feed, feed additive, food ingredient, water additive, water-mixed additive, consumable solution, consumable spray additive, consumable solid, consumable gel, injection, or combinations thereof. The composition may be formulated and suitable for use as or in one or more of animal feed, feed additive, food ingredient, water additive, water-mixed additive, consumable solution, consumable spray additive, consumable solid, consumable gel, injection, or combinations thereof. The composition may be suitable and prepared for use as animal feed, feed additive, food ingredient, water additive, water-mixed additive, consumable solution, consumable spray additive, consumable solid, consumable gel, injection, or combinations thereof.

Compositions may include a carrier in which a bacterium or any such other components is suspended or dissolved. Such carrier(s) may be any solvent or solid or encapsulated in a material that is non-toxic to the inoculated animal and compatible with the organism. Suitable pharmaceutical carriers include liquid carriers, such as normal saline and other non-toxic salts at or near physiological concentrations, and solid carriers, such as talc or sucrose and which can also be incorporated into feed for farm animals. When used for administering via the bronchial tubes, the composition is preferably presented in the form of an aerosol. A dye may be added to the compositions hereof, including to facilitate checking or confirming whether an animal has Ingested or breathed in the composition.

When administering to animals, including farm animals, administration may include orally or by injection. Oral administration can include by bolus, tablet or paste, or as a powder or solution in feed or drinking water. The method of administration will often depend on the species being feed or administered, the numbers of animals being fed or administered, and other factors such as the handling facilities available and the risk of stress for the animal.

The dosages required will vary and need be an amount sufficient to induce an immune response or to effect a biological or phenotypic change or response expected or desired. Routine experimentation will establish the required amount. Increasing amounts or multiple dosages may be implemented and used as needed.

In an embodiment, bacterial strains are administered in doses indicated as CFU/g or colony forming units of bacteria per gram. In an embodiment, the dose is in the range of 1×10³ to 1×10⁹ CFU/g. In an embodiment, the dose is in the range of 1×10³ to 1×10⁷. In an embodiment, the dose is in the range of 1×10⁴ to 1×10⁶. In an embodiment, the dose is in the range of 5×10⁴ to 1×10⁶. In an embodiment, the dose is in the range of 5×104 to 6×10⁵. In an embodiment, the dose is in the range of 7×10⁴ to 3×10⁵. In an embodiment, the dose is approximately 50K, 75K, 100K, 125K, 150K, 200K, 300K, 400K, 500K, 600K CFU/g.

Peptides for use in the present disclosure may include synthetic, recombinant or peptidomimetic entities. The peptides may be monomers, polymers, multimers, dendrimers, concatamers of various forms known or contemplated in the art, and may be so modified or multimerized so as to improve activity, specificity or stability. For instance, and not by way of limitation, several strategies have been pursued in efforts to increase the effectiveness of antimicrobial peptides including dendrimers and altered amino acids (Tam, J. P. et al (2002) Eur J Biochem 269 (3): 923-932; Janiszewska, J. et al (2003) Bioorg Med Chem Lett 13 (21):3711-3713; Ghadiri et al. (2004) Nature 369(6478):301-304; DeGrado et al (2003) Protein Science 12(4):647-665; Tew et al. (2002) PNAS 99(8):5110⁻⁵¹¹⁴; Janiszewska, J et al (2003) Bioorg Med Chem Lett 13 (21): 3711-3713). U.S. Pat. No. 5,229,490 to Tam discloses a particular polymeric construction formed by the binding of multiple antigens to a dendritic core or backbone.

In an aspect of the disclosure, the AMPs, such as, CAP 18 and variant CAP18 peptides of the disclosure may be attached to another molecule or may be labeled, including labeled with a detectable label. The label may include or may be selected from radioactive elements, enzymes, chemicals which fluoresce when exposed to ultraviolet light, and others. A number of fluorescent materials are known and can be utilized as labels. These include, for example, fluorescein, rhodamine, auramine, Texas Red, AMCA blue and Lucifer Yellow. The PGRN fragment including ND7/Pcgin can also be labeled with a radioactive element or with an enzyme. The radioactive label can be detected by any of the currently available counting procedures. The isotope may be selected from ³H, ¹⁴C, ³²P, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re. Enzyme labels are likewise useful, and can be detected by any of the presently utilized colorimetric, spectrophotometric, fluorospectrophotometric, amperometric or gasometric techniques. The enzyme may be conjugated to the PGRN fragment by reaction with bridging molecules such as carbodiimides, diisocyanates, glutaraldehyde and the like. Many enzymes which can be used in these procedures are known and can be utilized. The preferred are peroxidase, ß-glucuronidase, ß-D-glucosidase, ß-D-galactosidase, urease, glucose oxidase plus peroxidase and alkaline phosphatase.

In an aspect of the disclosure, the CAP 18 and variant CAP18 peptides of the disclosure may be covalently attached to another molecule or may be a fusion protein. Thus, conjugates or fusion proteins of the present disclosure, wherein the peptide of the present disclosure, or one or more peptide(s) of the present disclosure are conjugated or attached to other molecules or agents further include, but are not limited to peptides conjugated to a cell or pathogen targeting agent or sequence, toxin, immunomodulator, cytokine, cytotoxic agent, or one or more anti-bacterial, anti-parasitic or anti-viral agent or drug.

In an assay, diagnostic method or kit of the disclosure, a control quantity of the CAP 18 and variant CAP18 peptides of the disclosure or antibodies thereto, or the like may be prepared and labeled with an enzyme, a specific binding partner and/or a radioactive element, and may then be introduced into a cellular sample. After the labeled material or its binding partner(s) has had an opportunity to react with sites within the sample, the resulting mass may be examined by known techniques, which may vary with the nature of the label attached. In the instance where a radioactive label, such as the isotopes ³H, ¹⁴C, ³²p, ³⁵S, ³⁶Cl, ⁵¹Cr, ⁵⁷Co, ⁵⁸Co, ⁵⁹Fe, ⁹⁰Y, ¹²⁵I, ¹³¹I, and ¹⁸⁶Re are used, known currently available counting procedures may be utilized. In the instance where the label is an enzyme, detection may be accomplished by any of the presently utilized colorimetric, spectrophotometric, fluorospectrophotometric, amperometric or gasometric techniques known in the art.

In an embodiment, a diagnostic method of the present disclosure comprises examining a cellular sample or medium by means of an assay including an effective amount of an antibody or alternative binder that recognizes the CAP 18 and variant CAP18 peptides of the disclosure or a tag or label attached thereto. In an embodiment, the antibody may be in the form of Fab, Fab′, F(ab′)₂ or F(v) portions or whole antibody molecules.

The present disclosure further contemplates pharmaceutical compositions and therapeutic compositions useful in practicing the therapeutic methods of this disclosure. A subject pharmaceutical composition or therapeutic composition includes, in admixture, a pharmaceutically acceptable excipient (carrier) and one or more of PGRN fragment, ND7 or active variant thereof, as described herein as an active ingredient.

The preparation of pharmaceutical compositions and therapeutic compositions which contain one or more peptide(s) as the active ingredient(s) is well understood in the art. Such compositions may be prepared as liquid solutions or suspensions, such as for injectables. Solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

One or more peptide(s) can be formulated into the pharmaceutical composition or the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The peptide(s) may be prepared in pharmaceutical compositions, with a suitable and acceptable carrier and at a strength effective for administration by various means to a patient experiencing an adverse medical condition associated with pathogenic infection or bacterial infection or exposure to resistant bacteria, or exposure to a parasite or virus, or risk of any such exposure, or the specific need for the treatment thereof. The compositions may comprise one or more peptide alone or in combination with another agent, such as an anti-bacterial agent, anti-infective agent, immunomodulatory agent, etc. A variety of administrative techniques may be utilized, among them topical, enteral, and parenteral techniques. Administration may be via any suitable mode or method, such as oral, rectal, transmucosal, transdermal, subcutaneous, intravenous and intraperitoneal injections, catheterizations and the like. Average quantities of the peptides and/or agents may vary and in particular should be based upon the recommendations and prescription of a qualified physician or veterinarian.

The CAP peptide containing pharmaceutical compositions or therapeutic compositions may be administered intravenously, as by injection of a unit dose, for example, and may be administered via any suitable means including IM, IP, IV, orally, intranasally, by inhalation, transdermally, etc. The term “unit dose” when used in reference to a therapeutic composition of the present disclosure refers to physically discrete units suitable as unitary dosage for humans or other animal, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject or animal to be treated, the target location in or of the animal or subject, capacity of the system, such as the applicable immune system or digestive system to utilize the active ingredient, and CAP18 peptide-mediated anti-pathogenic activity desired. Precise amounts of active ingredient required to be administered depend on the judgment of the practitioner and are peculiar to each individual. Dosages may range from about 0.001 to 1, 0.01 to 10, 0.1 to 20, 0.5 to 50, preferably about 0.5 to about 10, and more specifically one to several, milligrams of active ingredient per kilogram body weight of individual animal and depend on the route of administration. Suitable regimes for initial administration and subsequent administration are also variable, but are typified by an initial administration followed by repeated doses at one or more hour intervals by a subsequent administration.

When administering to animals, including farm animals, administration may be orally or by injection. Oral administration can include by bolus, tablet or paste, or as a powder or solution in feed or drinking water. The method of administration will often depend on the species being treated, the numbers needing treatment, and other factors such as the handling facilities available and the risk of stress for the animal.

Pharmaceutical compositions for oral administration may be in tablet, capsule, powder or liquid form. A tablet may comprise a solid carrier such as gelatin or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. For intravenous, injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's injection. Preservatives, stabilizers, buffers, antioxidants and/or other additives may be included, as required.

A composition may be administered alone or in combination with other treatments, therapeutics or agents, either simultaneously or sequentially dependent upon the condition to be treated.

Compositions for treating topical infections or contaminations comprise an effective amount of at least one variant CAP18 peptide according to the disclosure and a carrier for delivering at least one peptide to the infected or contaminated skin, coat, or external surface of an animal, including livestock. The mode of application for the lytic enzyme includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. A mode of delivery of the carrier containing the therapeutic agent includes, but is not limited to a smear, spray, a time-release patch, a liquid absorbed wipe, and combinations thereof. The lytic enzyme may be applied to a bandage either directly or in one of the other carriers. The bandages may be sold damp or dry, wherein the enzyme is in a lyophilized form on the bandage. This method of application is most effective for the treatment of infected skin. The carriers of topical compositions may comprise semi-solid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament. Polymer thickeners that may be used include those known to one skilled in the art, such as hydrophilic and hydroalcoholic gelling agents frequently used in the cosmetic and pharmaceutical industries. CARBOPOL™ is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. KLUCEL™ is a cellulose polymer that is dispersed in water and forms a uniform gel upon complete hydration. Other specific gelling polymers include hydroxyethylcellulose, cellulose gum, MVE/MA decadiene crosspolymer, PVM/MA copolymer, or a combination thereof.

A composition comprising a peptide(s) can be administered in the form of a candy, chewing gum, lozenge, troche, tablet, a powder, an aerosol, a liquid, a liquid spray, or toothpaste for the prevention or treatment of bacterial infections associated with upper respiratory tract illnesses. The lozenge, tablet, or gum into which the lytic enzyme/polypeptide(s) is added may contain sugar, corn syrup, a variety of dyes, non-sugar sweeteners, flavorings, any binders, or combinations thereof. Similarly, any gum-based products may contain acacia, carnauba wax, citric acid, cornstarch, food colorings, flavorings, non-sugar sweeteners, gelatin, glucose, glycerin, gum base, shellac, sodium saccharin, sugar, water, white wax, cellulose, other binders, and combinations thereof. Lozenges may further contain sucrose, cornstarch, acacia, gum tragacanth, anethole, linseed, oleoresin, mineral oil, and cellulose, other binders, and combinations thereof. Sugar substitutes can also be used in place of dextrose, sucrose, or other sugars.

Compositions comprising lytic enzymes, or their peptide fragments can be directed to the mucosal lining, where, in residence, they kill colonizing disease bacteria. The mucosal lining, as disclosed and described herein, includes, for example, the upper and lower respiratory tract, eye, buccal cavity, nose, rectum, vagina, periodontal pocket, intestines and colon. Due to natural eliminating or cleansing mechanisms of mucosal tissues, conventional dosage forms are not retained at the application site for any significant length of time.

It may be advantageous to have materials which exhibit adhesion to mucosal tissues, to be administered with one or more phage enzymes and other complementary agents over a period of time. Materials having controlled release capability are particularly desirable, and the use of sustained release mucoadhesives has received a significant degree of attention. J. R. Robinson (U.S. Pat. No. 4,615,697, incorporated herein by reference) provides a review of the various controlled release polymeric compositions used in mucosal drug delivery. The patent describes a controlled release treatment composition which includes a bioadhesive and an effective amount of a treating agent. The bioadhesive is a water swellable, but water insoluble fibrous, crosslinked, carboxy functional polymer containing (a) a plurality of repeating units of which at least about 80 percent contain at least one carboxyl functionality, and (b) about 0.05 to about 1.5 percent crosslinking agent substantially free from polyalkenyl polyether. While the polymers of Robinson are water swellable but Insoluble, they are crosslinked, not thermoplastic, and are not as easy to formulate with active agents, and into the various dosage forms, as the copolymer systems of the present application. Micelles and multilamillar micelles may also be used to control the release of enzyme.

Other approaches Involving mucoadhesives which are the combination of hydrophilic and hydrophobic materials, are known. ORAHESIVE®. from E. R. Squibb & Co is an adhesive which is a combination of pectin, gelatin, and sodium carboxymethyl cellulose in a tacky hydrocarbon polymer, for adhering to the oral mucosa. However, such physical mixtures of hydrophilic and hydrophobic components eventually fall apart. In contrast, the hydrophilic and hydrophobic domains in this application produce an insoluble copolymer. U.S. Pat. No. 4,948,580, also incorporated by reference, describes a bioadhesive oral drug delivery system. The composition includes a freeze-dried polymer mixture formed of the copolymer poly(methyl vinyl ether/maleic anhydride) and gelatin, dispersed in an ointment base, such as mineral oil containing dispersed polyethylene. U.S. Pat. No. 5,413,792 (incorporated herein by reference) discloses paste-like preparations comprising (A) a paste-like base comprising a polyorganosiloxane and a water soluble polymeric material which are specifically present in a ratio by weight from 3:6 to 6:3, and (B) an active ingredient. U.S. Pat. No. 5,554,380 claims a solid or semisolid bioadherent orally ingestible drug delivery system containing a water-in-oil system having at least two phases. One phase comprises from about 25% to about 75% by volume of an internal hydrophilic phase and the other phase comprises from about 23% to about 75% by volume of an external hydrophobic phase, wherein the external hydrophobic phase is comprised of three components: (a) an emulsifier, (b) a glyceride ester, and (c) a wax material. U.S. Pat. No. 5,942,243 describes some representative release materials useful for administering antibacterial agents, which are incorporated by reference.

Therapeutic or pharmaceutical compositions can also contain polymeric mucoadhesives including a graft copolymer comprising a hydrophilic main chain and hydrophobic graft chains for controlled release of biologically active agents. The graft copolymer is a reaction product of (1) a polystyrene macromonomer having an ethylenically unsaturated functional group, and (2) at least one hydrophilic acidic monomer having an ethylenically unsaturated functional group. The graft chains consist essentially of polystyrene, and the main polymer chain of hydrophilic monomeric moieties, some of which have acidic functionality. The weight percent of the polystyrene macromonomer in the graft copolymer is between about 1 and about 20% and the weight percent of the total hydrophilic monomer in the graft copolymer is between 80 and 99%, and wherein at least 10% of said total hydrophilic monomer is acidic, said graft copolymer when fully hydrated having an equilibrium water content of at least 90%. Compositions containing the copolymers gradually hydrate by sorption of tissue fluids at the application site to yield a very soft jelly like mass exhibiting adhesion to the mucosal surface. During the period of time the composition is adhering to the mucosal surface, it provides sustained release of the pharmacologically active agent, which is absorbed by the mucosal tissue.

The compositions of this disclosure may optionally contain other polymeric materials, such as poly(acrylic acid), poly,-(vinyl pyrrolidone), and sodium carboxymethyl cellulose plasticizers, and other pharmaceutically acceptable excipients in amounts that do not cause deleterious effect upon mucoadhesivity of the composition.

The present disclosure naturally contemplates several means for preparation of the CAP 18 and variant CAP18 peptides of the disclosure, including synthetic methods and/or using known recombinant techniques, and the disclosure is accordingly intended to cover such recombinant or synthetic preparations within its scope. The determination of the amino acid sequences disclosed herein facilitates the reproduction of the peptides by any of various synthetic methods or any known recombinant techniques. Accordingly, the disclosure extends to expression vectors comprising nucleic acid encoding the peptides of the present disclosure for expression in host systems by recombinant DNA techniques, and to the resulting transformed hosts.

In an embodiment, nucleic acid encoding one or more of the CAP 18 peptides of the disclosure are provided. The disclosure also relates to a recombinant DNA molecule, recombinant nucleic acid, or cloned gene, or a degenerate variant thereof, preferably a nucleic acid molecule, in particular a recombinant DNA molecule or cloned gene, encoding the amino acid of CAP 18 and variant CAP18 peptide(s) of the disclosure. In a particular embodiment, the recombinant DNA molecule, recombinant nucleic acid, or a degenerate variant thereof, preferably a nucleic acid molecule, encodes a CAP 18 and variant CAP18 peptide(s) of the disclosure. DNA sequences may be expressed by operatively linking them to an expression control sequence in an appropriate expression vector and employing that expression vector to transform an appropriate unicellular host. Such operative linking of a DNA sequence of this disclosure to an expression control sequence, includes, if not already part of the DNA sequence, the provision of an initiation codon, ATG, in the correct reading frame upstream of the DNA sequence.

A wide variety of host/expression vector combinations may be employed in expressing the DNA sequences of this disclosure. Useful expression vectors, for example, may consist of segments of chromosomal, non-chromosomal and synthetic DNA sequences. Suitable vectors may depend on the animal or cell type selected for expression and will be available and known to one skilled in the art. Any of a wide variety of expression control sequences—sequences that control the expression of a DNA sequence operatively linked to it—may be used in these vectors to express the DNA sequences of this disclosure.

A wide variety of unicellular host cells are also useful in expressing the DNA sequences of this disclosure. These hosts may include well known eukaryotic and prokaryotic hosts, such as strains of E. coli, Pseudomonas, Bacillus, Streptomyces, fungi such as yeasts, and animal cells, human cells and plant cells in tissue culture.

### Direct Fed Microbials

Direct fed microbials (DFMs), often also called probiotics, are microorganisms which colonize the gastrointestinal tract of an animal and provide some beneficial effect to that animal. The microorganisms can be bacterial species, for example those from the genera Bacillus, Lactobacillus, Lactococcus, and Enterococcus. The microorganisms can also be yeast or even molds. The microorganisms can be provided to an animal orally or mucosally or, in the case of birds, provided to a fertilized egg, i.e. in ovo.

The beneficial activity provided by a DFM can be through the synthesis and secretion of vitamins or other nutritional molecules needed for a healthy metabolism of the host animal. A DFM can also protect the host animal from disease, disorders, or clinical symptoms caused by pathogenic microorganisms or other agents. For example, the DFM may naturally produce factors having inhibitory or cytotoxic activity against certain species of pathogens, such as deleterious or disease-causing bacteria.

Probiotics and DFMs provide an attractive alternative or addition to the use and application of antibiotics in animals. Antibiotics can promote resistant or less sensitive bacteria and can ultimately end up in feed products or foods consumed by other animals or humans. DFMs are characterized as being generally safe (even denoted Generally Regarded as Safe (GRAS) and most are not naturally resistant to antibiotics.

### DFMs as Delivery System

in some instances, the DFM may not be able to produce such factors in sufficient quantity to reduce infection of the host with the pathogen, or the factors may affect only a limited set of pathogens, leaving the host vulnerable to other pathogens. Strains suitable as DFMs can provide an attractive and useful starting point for applications to produce or generate biomolecules and heterologous proteins, including as a live delivery system for synthesis and delivery of molecules or proteins with wide applications including in therapy and in animal health.

These direct feed strains have applicability as a delivery system which can constantly deliver useful therapeutic molecules and biomolecules, such as anti-infective molecules, directly to the host, such as to the gastrointestinal tract, where pathogenic bacteria are replicating in the host. The gastrointestinal system is also often a point of entry of the pathogen into the host. Preferably, the delivery system is a live genetically-modified microorganism, such as a bacterium, which can reproduce in—and even colonize in some instances—a host and directly deliver therapeutic molecules and biomolecules, such as antiinfective, antipathogenic or one or more of the antibacterial polypeptides of this disclosure to reduce the number of, or block the entry of, a pathogen. These bacterial strains provide improved delivery platforms and systems, including suitable vectors and nucleic acid-based systems for rapid and effective expression of heterologous proteins or genes of interest and robust generation of numerous vehicles using a single platform.

### DFMs as Production System

Recombinant protein production in microbial cells is an important aspect of the modem biotechnological industry. Intracellular expression of heterologous proteins in host cells is widely utilized and such proteins are isolated from a culture of producing host cells. Biomolecules or heterologous proteins can be expressed from plasmids transfected into bacterial cells or from encoding sequence(s) integrated in the host bacteria genome.

In addition, recent achievements in secretory expression of recombinant proteins have encouraged both the scientific and industrial communities to apply and implement bacteria with a secretory ability for protein production. Using secretory-type host cells, synthesized target biomolecules and proteins are secreted directly and accumulated in the extracellular medium, which provides cost-effective downstream purification processing. Further, this can permit production and isolation of target biomolecules and proteins without the need or requirement for lysing the host cells. Also, secretory expression of recombinant proteins prevents accumulation of target biomolecules heterologous proteins within host cells, which can limit cell growth and production, lead to cell toxicity and result in incorrect protein folding (Mergulhao, F. J.; Summers, D. K.; Monteiro, G. A. (2005) Biotechnol Adv 23(3):177-202; Song, Y.; Nikoloff, J. M.; Zhang, D. (2015) J Microbiol Biotechnol 25(7): 963-77).

Bacillus subtilis—Strain 105

Bacillus subtilis is a Gram-positive model bacterium which is widely used for industrial production of recombinant proteins such as alpha-amylase, protease, lipase, and other industrial enzymes. Because of the ability of the bacteria to produce large amounts of a target protein, and also to secrete large amounts of a target protein into the culture medium, and the availability of a low-cost downstream production and purification process, over 60% of commercial industrial enzymes are produced in Bacillus subtilis and relative Bacillus species (Schallmey, M.; Singh, A.; Ward, O. P. (2004) 50 (1): 1-17). In contrast to the frequently used recombinant protein expression host Escherichia coli, Bacillus subtilis has no risk of endotoxin contamination and has been certificated as a GRAS (generally regarded as safe) organism by the FDA, which makes it a choice for food-grade and pharmaceutical protein production.

B. subtilis strains, particularly strain 105, provides a Bacillus subtilis expression system which can be modified and engineered to produce high levels of at least one or a multiplicity of biomolecules or heterologous proteins, including in instances as surface-displayed or secreted molecules.

Bacillus subtilis strain ELA191105, also denoted strain 105, corresponds to ATCC deposit PTA-126786. Strain 105 is described and detailed as a genetically modified strain for live delivery or production in U.S. Ser. No. 63/247,271 (filed Sep. 11, 2021), 63/247,273 (filed Sep. 22, 2021) and 63/247,400 (filed Sep. 23, 2021), which applications are incorporated herein by reference.

These applications describe native bacterial promoters, signal sequences suitable for expression and vectors and bacterial genome sites/genes for integration to generate stable modified strains, as well as modifications to strain 105 to improve expression.

### Lactobacillus Reuteri Strains 3630 and 3632

Lactobacillus reuteri strains 3630 and 3632 are described and detailed as novel strains suitable as DFMs, including in combination, and also as suitable strains for genetic modification and as live delivery or production strains.

Lactobacillus reuteri strain 3632 was deposited on 19 Jun. 2020 according to the Budapest Treaty in the ATCC Patent Depository and assigned ATCC Patent Deposit Number PTA-126788. Lactobacillus reuteri strain 3630 was deposited on 19 Jun. 2020 in the ATCC Patent Depository and assigned ATCC Patent Deposit Number PTA-126787.

The L reuteri strains 3630 and 3632 are described and detailed as probiotic strains in Probiotic Compositions Comprising Lactobacillus Reuteri Strains and Methods of Use PCT/US2020/016668 filed Feb. 4, 2020, published as WO 2020/163398 Aug. 13, 2020. Priority parent is 62/801,307 filed Feb. 5, 2019. Corresponding US publications are US 2022/0088094 published Mar. 24, 2022 and US 2022/0125860 published Apr. 28, 2022. All of the foregoing patent applications are incorporated herein by reference in their entireties.

A live delivery system based on L. reuteri strain 3630 or 3632 is described and detailed in A Genetically Modified Lactobacillus and Uses Thereof, PCT/US2020/016522 filed Feb. 4, 2020, published as WO 2020/163284 Aug. 13, 2020. Priority parent is 62/801,307 filed Feb. 5, 2019. All of the foregoing patent applications are incorporated herein by reference in their entireties. This application describes native bacterial promoters, signal sequences suitable for expression and vectors and bacterial genome sites/genes for integration to generate stable modified strains.

A method of the present disclosure includes administering to an animal a unicellular host capable of heterologously expressing at least one of the engineered polypeptides disclosed herein. Any of the bacterial production systems disclosed herein can be used to produce the engineered polypeptides of this disclosure in an animal. In an embodiment the unicellular host is transformed by a vector that comprises nucleic acid encoding the engineered polypeptide. In an embodiment the unicellular host includes a genome into which heterologous nucleic acid encoding the engineered polypeptide has been integrated. In an embodiment the nucleic acid comprises a recombinant DNA molecule, a recombinant nucleic acid, or cloned gene, or a degenerate variant thereof, encoding said engineered polypeptide. In an embodiment the unicellular host is administered to the animal by intranasal spray, by injection, as part of a direct fed microbial, or by oral administration.

It will be appreciated that other embodiments and uses will be apparent to those skilled in the art and that the present disclosure is not limited to these specific illustrative examples or specific embodiments.

### Example 1

CAP18 is a versatile peptide with antimicrobial activity against bacteria and viruses. CAP18 is an 18 kDa, pore forming, lipopolysaccharide (LPS)-binding antimicrobial peptide (37 aa) belonging to cathelicidin family of antimicrobial peptides. Originally isolated and characterized from neutrophils, CAP18 has antimicrobial activity against a variety of bacterial pathogens, including Staphylococcus aureus, Streptococcus pneumonia, Escherichia coil, Pseudomonas aeruginosa, Salmonella Typhimurium, Yersinia ruckeri, Aeromonas salmonicida, Campylobacter jejuni, Enterococcus faecalis, and Listeria monocytogenes. CAP18 is highly thermostable—known to retain full antimicrobial activity even after treatment at 90° C. for 30 minutes. In addition to antimicrobial activity, CAP18 potently binds LPS and has scavenges LPS to reduce inflammation. Thus, CAP18 not only has the potential to address multiple microbial etiologies in animal/human health, it can also serve as an LPS scavenger to reduce the potential for LPS-induced inflammatory responses.

CAP18 peptide (wild type peptide also herein designated as AMP01) has application and utility for multiple indications in livestock, poultry & aqua species. CAP18 demonstrates anti-bacterial and killing activity against various bacteria relevant to diseases in animals, particularly, Mannheimia haemolytica and Pasteurella multocida (BRD, cattle), E. coli (Colibacillosis, poultry), Salmonella (Salmonellosis, poultry), C. jejuni (Campylobacteriosis, poultry), P. salmonis (SRS, salmon). Activity against these bacteria is demonstrated including in a plate inhibition assay (FIGS. 2A and 2B). FIG. 2B shows plate assay inhibition of BRD pathogens M. haemolytica and P. mutlocida (MIC of 4-8 μg/ml). Cap18 peptide is active against viruses of significance for the animal industry, such as PRRSV (PRRS, swine) (FIG. 2C). CAP18 peptide is effective in inhibiting activity of parasites of significance in animals such as Giardia (Giardiosis, dogs) and Eimeria (Coccidiosis, poultry). FIG. 3 depicts activity of CAP18 peptide (AMP01) against Giardia.

A study was undertaken to design, generate and evaluate variant CAP18 peptides having enhanced anti-pathogen activity and improved stability and resistance to protease as candidates and peptides for use and application in controlling, alleviating or reducing colonization or infection by bacterial parasitic or viral pathogens.

CAP18 and CAP18 peptides as a potential therapeutic for Giardia dogs

### Methods

Test tube with a confluent layer of trophozoites is treated with AMP01 (100 μM). Light microscopy, counting of detached dead or alive (i.e. motile) trophozoites is then conducted. Formononetin (5 μM; FOR) is used for comparison as a positive control.

### Results

After 1 h, nearly complete detachment of trophozoites in AMP01 or FOR treated tubes was observed. Trophozoites detached with FOR were pear-shaped and motile, whereas trophozoites detached with AMP01 were crumbled, shrunken and immotile, i.e. most probably already dead. The changes in morphology were still visible one day after the treatment and are depicted in FIG. 3 (photographs all taken at 100×). After 1 d, the trophozoites in the control tube (FIG. 3A) were fully alive (and overgrowing), as expected. The trophozoites in the FOR treated tube were still pear shaped, but with a grainy cytosol and most likely dead (FIG. 35). The trophozoites in the AMP01 treated tube were dead, shrunken and started to disintegrate (FIG. 3C). The different sizes of the trophozoites can be noted by comparing the white circles. Similar effects were observed using cytotoxic anti-variant surface protein antibodies (Hemphil et al. 1996).

Data comparing AMP01 (wild type CAP 18 peptide (SEQ ID NO: 1) with various other agents is tabulated in Table 3. NTZ corresponds to nitazoxanide. ALB corresponds to albendazole.

TABLE 3

| | | Conc | % of | % of | |
|---|---|---|---|---|---|
| Plate | Probe | (uM or %) | control MV | control SD | Inhibition (Y/N) |
| G7 | AMP01 | 100 | 1.0 | 0.8 | Y |
| G7 | AMP01 | 10 | 141.1 | 33.6 | N |
| G7 | Scath-2 | 100 | 130.0 | 33.0 | N |
| G7 | Scath-2 | 10 | 137.1 | 29.8 | N |
| G7 | Hep-1 | 100 | 58.2 | 18.0 | N |
| G7 | Hep-1 | 10 | 111.6 | 38.5 | N |
| G7 | MET | 10 | 0.9 | 0.7 | Y |
| G7 | NTZ | 10 | 0.6 | 0.5 | Y |
| G7 | ALB | 10 | 1.5 | 0.7 | Y |

### CAP18 (AMP01)—Effect on Eimira

The effect of Cap 18 peptides and any variants thereof is tested on Eimeria. An exemplary study is provided with results depicted in FIG. 4. Three independent experiments were conducted with CAP18 peptides—designated Cap18a, Cap18b and Cap18c. The CAP18 peptides were compared with monensin as a positive control. Three independent experiments with monensin (designated Mona, Monb and Monc).

### CAP18 (AMP01)—Effect on PRRSV

Antibacterial peptide CAP18 was evaluated for activity inhibiting virus, particularly its antiviral effect on porcine reproductive and respiratory syndrome virus (PRRSV). Results are provided in Figure S. AMP01 polypeptide was evaluated at 1:2 and 1:4 diluted and shown to be effective.

### Antimicrobial Effects of CAP18 Peptides

Antimicrobial effects of CAP18 peptides (or variants thereof) are evaluated against pathogenic bacteria. Isolates of avian pathogenic E. coli (APEC), C. jejuni (Campylobacter) and S. Typhimurium (Salmonella) were tested for antimicrobial effect of CAP18 peptides, including in comparison with other agents (FIG. 2).

Antimicrobial Effect of AMP01 against BRD Pathogens

Antimicrobial effect of CAP18 against BRD pathogens M. haemolytica and P. multocida was evaluated. Clearing on the bacteria plate where the peptide is applied demonstrates antimicrobial effect or antibacterial effect of the peptide against the test organism/microbe/bacteria. Results are shown in FIG. 25 and data not shown.

### Example 2

### CAP 18 Variants

A study was undertaken to design CAP18 variants with no cytokine stimulatory activity and with potential for protease resistance. The primary design principles included the use of Lys instead of Arg, as well as the placement or Pro at the carboxyl side of Arg to avoid hydrolysis of trypsin. In addition, abandoning of Trp and Phe was utilized to prevent the hydrolysis of chymotrypsin. Predicted chymotrypsin sites are depicted in FIG. 6A. Predicted trypsin sites are depicted in FIG. 68. Various peptide amino acid mutations for consideration as reported are depicted in FIG. 6C.

CAP18 variant peptides having single amino acid changes for initial evaluation were designed and are shown in the Summary above and in FIG. 60. These include proline mutations, R->K mutations for trypsin, and F->I and L->I mutations for chymotrypsin. The various single amino acid mutation CAP18 variants were evaluated for maintenance of antibacterial/antimicrobial killing capability under different conditions. These studies were conducted to identify mutations which contribute to or provide enhanced thermotolerance and protease resistance, for example against proteinase K or trypsin. In each instance the mutant CAP18 peptides were subjected to a condition and then killing was assessed in a MIC assay. The MIC assays were conducted using the following protocol.

Testing of CAP18 variants for protease resistance using Escherichia coli Dh5alpha as an indicator organism. CAP18 variants were synthesized from GenScript, Inc, and solubilized in sterile purified water to a final concentration of 4 mg/ml. E. coli DH5alpha was grown overnight in 10 ml LB broth. Ten microliters of each CAP18 variant was treated with 2 μl (or 2 μl of the 10-fold diluted trypsin) of trypsin (0.25%, Sigma Aldrich) and incubated at 37° C. for 2 or 5 minutes in a PCR machine. An MIC assay was set up using trypsin-treated CAP18 variants in a 96-well plate. Briefly, the peptides were serially diluted 2-fold in LB broth to achieve a final volume of 50 μl in each well and 50 μl of the E. coli DH5alpha culture adjusted to a concentration of 2×106 cells/ml was added into each well. Appropriate controls (media only control, no CAP18 control) were included on the same plate. The plates were incubated at 37° C. for 24-48 hours and MIC for each variant was recorded. The experiment was repeated 3 times.

Alternative MIC assays or assays or methods for determination of bacterial or microbial or infectious agent killing known and available in the art may also be utilized. One such approach for antimicrobial susceptibility testing is in accordance with the following.

Antimicrobial susceptibility testing (MIC testing): The minimum inhibitory concentrations (MICs) of the AMPs were measured in 96-well microtiter plates according the Clinical and Laboratory Standards institute (CLSI, formerly National Committee for Clinical Laboratory Standards [NCCLS]) (Wikler M A, et al. (2009) Methods for dilution antimicrobial susceptibility tests for bacteria that grow aerobically; approved standard eighth edition. Clinical and Laboratory Standards institute). Briefly, liquid Mueller-Hinton-li medium containing increasing concentrations of AMPs is inoculated with a defined number of cells (approx. 105 CFUs/ml) in 96-well microtiter plates (polypropylene), whereas each plate also includes a positive growth control and a negative control (sterile control). The range of peptide concentrations analyzed was 0.125±64 μg/ml for the high purity peptides and 0.06±32 μg/ml for peptides of the variant library. After incubation, the MIC is determined by the lowest concentration showing no visible growth. All plates were incubated for 16±20 hours. Variations to this testing and assay can readily be made and utilized by one skilled in the art, including as applicable depending on the bacteria and species being tested or evaluated.

### A Thermostable Antimicrobial Peptide

Thermotolerance was evaluated at 90 for 5 minutes and bacterial killing assessed (MIC, ug/ml). CAP18 variants were synthesized from GenScript, Inc, and solubilized in sterile purified water to a final concentration of 4 mg/ml. E. coli DH5 alpha was grown overnight in 10 ml LB broth. Ten microliters of each CAP18 variant was incubated at 98° C. for 5 minutes in a PCR machine. A MIC assay was set up using heat treated CAP18 variants in a 96-well plate. Briefly, the peptides were serially diluted 2-fold in LB broth to achieve a final volume of 501 in each well and 50 μl of the E. coli DH5alpha culture adjusted to a concentration of 2×10⁶ cells/ml was added into each well. Appropriate controls (media only control, no CAP18 control) were included on the same plate. The plates were incubated at 37° C. for 24-48 hours and MIC for each variant was recorded. The experiment was repeated 3 times. The Results are provided below in TABLE 4.

TABLE 4

| CAP18 mutant | Thermotolerance at 90 for 5 minutes (MIC, ug/ml) |
|---|---|
| C1 | 200 |
| C2 | 100 |
| C3 | >400 |
| C4 | 400 |
| C5 | >400 |
| C6 | 1200 |
| C7 | 200 |
| C8 | 100 |
| C9 | 50 |
| C10 | 100 |
| C11 | 200 |
| C12 | 100 |
| C13 | 200 |
| C14 | 100 |
| C15 | 200 |
| C16 | 100 |
| C17 | >400 |
| WT | 200 |

CAP18 peptide variants single mutants were evaluated for resistance to proteinase K. Peptides were subjected to proteinase K treatment and then assessed for bacterial cell killing (against E. coli) in a MIC assay.

CAP18 variants were synthesized from GenScript, Inc, and solubilized in sterile purified water to a final concentration of 4 mg/ml. E. coli DH5alpha was grown overnight in 10 ml LB broth. Ten microliters of each CAP18 variant was treated with 2 μl of proteinase K (Qjagen, >600 mAU/ml) and incubated at 37° C. for 2 or 5 minutes in a PCR machine. The samples were then incubated at 98° C. for 5 minutes to inactivate proteinase K. An MIC assay was set up using proteinase K-treated CAP18 variants in a 96-well plate. Briefly, the peptides were serially diluted 2-fold in LB broth to achieve a final volume of 50 μl in each well and 50 μl of the E. coli DH5alpha culture adjusted to a concentration of 2×10⁶ cells/ml was added into each well. Appropriate controls (media only control, no CAP18 control) were included on the same plate. The plates were incubated at 37° C. for 24-48 hours and MIC for each variant was recorded. The experiment was repeated 3 times.

The results are provided below in TABLE 5.

TABLE 5

| CAP18 mutant | Resistance to proteinase K (MIC, ug/ml) |
|---|---|
| C1 | >400 |
| C2 | >400 |
| C3 | >400 |
| C4 | >400 |
| C5 | >400 |
| C6 | >400 |
| C7 | >400 |
| C8 | 400 |
| C9 | 400 |
| C10 | 400 |
| C11 | >400 |
| C12 | 200 |
| C13 | >400 |
| C14 | >400 |
| C15 | >400 |
| C16 | >400 |
| C17 | >400 |
| WT | >400 |

CAP18 peptide variants single mutants were evaluated for resistance to trypsin. Peptides were subjected to trypsin protease treatment and then assessed for bacterial cell killing (against E. coli) in a MIC assay.

CAP18 variants were synthesized from GenScript, Inc, and solubilized in sterile purified water to a final concentration of 4 mg/ml. E. coli DH5alpha was grown overnight in 10 ml LB broth. Ten microliters of each CAP18 variant was treated with 2 μl (or 2 μl of the 10-fold diluted trypsin) of trypsin (0.25%, Sigma Aldrich) and incubated at 37° C. for 2 or 5 minutes in a PCR machine. The samples were then incubated at 98° C. for 5 minutes to inactivate trypsin. An MIC assay was set up using trypsin-treated CAP18 variants in a 96-well plate. Briefly, the peptides were serially diluted 2-fold in LB broth to achieve a final volume of 50 μl in each well and 50 μl of the E. coli DH5alpha culture adjusted to a concentration of 2×10⁶ cells/ml was added into each well. Appropriate controls (media only control, no CAP18 control) were included on the same plate. The plates were incubated at 37° C. for 24-48 hours and MIC for each variant was recorded. The experiment was repeated 3 times. The results are depicted in TABLE 6.

TABLE 6

| | SEQ | Resistance | |
|---|---|---|---|
| CAP18 | ID | to trypsin | |
| mutant | NO: | (MIC, ug/ml) | Mutation_Sequence |
| C1 | 2 | 100 | GLRPRLRKFRNKIKEKLKKIGQKIQGLLPKLAPRTDY |
| C2 | 3 | >400 | GLRKRLRPFRNKIKEKLKKIGQKIQGLLPKLAPRTDY |
| C3 | 4 | >400 | GLRKRLRKFRPKIKEKLKKIGQKIQGLLPKLAPRTDY |
| C4 | 5 | >400 | GLRKRLRKFRNKIKPKLKKIGQKIQGLLPKLAPRTDY |
| C5 | 6 | 400 | GLRKRLRKERNKIKEKLKKIGQKIQGLLPKPAPRTDY |
| C6 | 7 | >400 | GLRKRLRKFRNKIKEKLKKIGQKIQGLLPKLAPRPDY |
| C7 | 8 | 50 | GLKKRLRKFRNKIKEKLKKIGQKIQGLLPKLAPRTDY |
| C8 | 9 | 50 | GLRKKLRKFRNKIKEKLKKIGQKIQGLLPKLAPRTDY |
| C9 | 10 | 400 | GLRKRLKKFRNKIKEKLKKIGQKIQGLLPKLAPRTDY |
| C10 | 11 | 50 | GLRKRLRKFKNKIKEKLKKIGQKIQGLLPKLAPRTDY |
| C11 | 12 | 200 | GLRKRLRKFRNKIKEKLKKIGQKIQGLLPKLAPKTDY |
| C12 | 13 | 100 | GLRKRLRKIRNKIKEKLKKIGQKIQGLLPKLAPRTDY |
| C13 | 14 | 100 | GIRKRLRKFRNKIKEKLKKIGQKIQGLLPKLAPRTDY |
| C14 | 15 | 50 | GLRKRIRKFRNKIKEKLKKIGQKIQGLLPKLAPRTDY |
| C15 | 16 | 50 | GLRKRLRKFRNKIKEKIKKIGQKIQGLLPKLAPRTDY |
| C16 | 17 | 50 | GLRKRLRKFRNKIKEKLKKIGQKIQGLLPKIAPRTDY |
| WT; 1 | 1 | 25 | GLRKRLRKFRNKIKEKLKKIGQKIQGLLPKLAPRTDY |

### Example 3

Peptides incorporating various mutations assessed above were generated, including wherein multiple mutations are incorporated.

Particular peptides are as follows. Mutations or amino acid changes from the wild type CAP18 (AMP01) sequence are shown in bold and underlined.

>cap18_R2Kv3_NTinh
(SEQ ID NO: 18)
CWTKSIPPKPCGLRKRLKKIKNKIKEKLKKIGQKIQGLLPKLAPRTDY
>cap18_hphobic2_NTinh
(SEQ ID NO: 19)
CWTKSIPPKPCGLRKILKKIKNKIKEKLKKIGQKIQGLLPKLAPRTDY
>cap18_R2Kv2_NTinh
(SEQ ID NO: 20)
CWTKSIPPKPCGLRKKLKKIRNKIKEKLKKIGQKIQGLLPKLAPRTDY
>cap18_R2Kv4_NTinh
(SEQ ID NO: 21)
CWTKSIPPKPCGLRKRLRKIKNKIKEKLKKIGQKIQGLLPKLAPKTDY
>cap18_CRKP1_NTinh
(SEQ ID NO: 22)
CWTKSIPPKPCGCRKPLRKIRNKIKEKLKKIGQKIQGLLPKLAPRTDY
>cap18_CRKP2_NTinh
(SEQ ID NO: 23)
CWTKSIPPKPCGCRKPCRKIRNKIKEKLKKIGQKIQGLLPKLAPRTDY

The sequence CWTKSIPPKPC shown above in bold is an N terminal sequence that can facilitate thermotolerance/thermostability and effectiveness of the peptide. Alternative variant peptides only without the N terminal sequence are contemplated.

Other variant CAP18 peptides have been evaluated and are provided and detailed herein, including in the description and specification above. These include CAP18 variants with enhanced protease resistance versus or in comparison to the wild type CAP18 (SEQ ID NO:1), but which may not have as enhanced resistance as the above particular peptides SEQ ID NO:s 18-23. These peptides may also have alternative functions or activities that are applicable or useful against one or more pathogen(s).

Some CAP18 variants did not evidence activity or protease resistance which was comparable at least to the wild type CAP18 peptide. These evidenced that the assays were operable and applicable and provided useful information in designing variant CAP18 peptides. Some peptides included alternative N terminal or C terminal tags or additional sequence (shown in bold). Variant amino acids compared to wild type sequence are shown in bold and underlined.

>cap18_cyc1
(SEQ ID NO: 43)
CGGGLRKRLRKIRNKIKEKLKKIGQKIQGLLPKLAPRTDYGGC
>cap18_cyc2
(SEQ ID NO: 44)
CGGSGLRKRLRKIRNKIKEKLKKIGQKIQGLLPKLAPRTDYGSGGC
>cap18_cycdimer
(SEQ ID NO: 45)
CGGGLRKRLRKIRNKIKEKLKKIGQKIQGLLPKLAPRTDYGAGGGLRKR
LRKFRNKIKEKLKKIGQKIQGLLPKLAPRTDYGGC
>cap18_NTinh
(SEQ ID NO: 46)
CWTKSIPPKPCGLRKRLRKIRNKIKEKLKKIGQKIQGLLPKLAPRTDY
>cap18_NTCTinh
(SEQ ID NO: 47)
CWTKSIPPKPCGLRKRLRKIRNKIKEKLKKIGQKIQGLLPKLAPRTDYC
WTKSIPPKPC
>cap18_CRKP3
(SEQ ID NO: 48)
GCRKPCRKPRNKIKEKLKKIGQKIQGLLPKLAPRTDY

Numerous variant CAP18 peptides were tested to determine and assess their resistance to protease. In each assessment, the variant CAP18 peptides were compared to wild type CAP18 rabbit sequence (GLRKRLRKFRNKIKEKLKKIGQKIQGLLPKLAPRTDY (SEQ ID NO:1)). Peptides were evaluated using trypsin diluted 1:10, 1:2 and undiluted. Experiments were conducted in duplicate. In each instance minimal inhibitory concentration (MIC) of the peptide was determined in the presence of the applicable diluted or undiluted trypsin and in the absence of trypsin. The results are tabulated below. Experiments l and 2 are duplicate assessments with 1:10 diluted trypsin. Experiments 3 and 4 are duplicate assessments with 1:2 diluted trypsin. Experiments 5 and 6 are duplicate assessments with undiluted trypsin.

Variant CAP 18 peptide designated as cap18_R2Kv3_NTinh having peptide sequence CWTKSIPPKPCGLRKRLKKIKNKIKEKLKKIGQKIQGLLPKLAPRTDY (SEQ ID NO: 18) was effective at the lowest concentration (lowest MIC) and was the most protease resistant in all experiments.

Variant CAP 18 peptide designated as cap18_hphobic2_NTinh having peptide sequence CWTKSIPPKPCGLRKILKKIKNKIKEKLKKIGQKIQGLLPKLAPRTDY (SEQ ID NO: 19) showed better activity (lower MIC) and protease resistance, compared to WT CAP18.

Variant CAP 18 peptide designated as cap18_R2Kv4_NTinh having peptide sequence CWTKSIPPKPCGLRKRLRKIKNKIKEKLKKIGQKIQGLLPKLAPKTDY (SEQ ID NO: 21) showed better activity (lower MIC) and protease resistance, compared to WT CAP18.

Variant CAP 18 peptide designated as cap18_CRKP1_NTinh having peptide sequence CWTKSIPPKPCGCRKPLRKIRNKIKEKLKKIGQKIQGLLPKLAPRTDY (SEQ ID NO: 22) showed better activity (lower MIC) and protease resistance, compared to WT CAP18.

Variant CAP 18 peptide designated as cap18_R2Kv2.NTinh having peptide sequence CWTKSIPPKPCGLRKKLKKIRNKIKEKLKKIGQKIQGLLPKLAPRTDY (SEQ ID NO: 20) showed better activity (lower MIC) and protease resistance, compared to WT CAP18.

Notably, these variant CAP18 peptides all demonstrated an improved MIC, having greater bacterial killing activity on a concentration basis, and also enhanced protease resistance compared to wild type CAP18 peptide. Bacterial killing and improved MIC was demonstrated even in undiluted trypsin was observed.

TABLE 7
Experiment 1-1:10 diluted trypsin

| | | | MIC with trypsin | |
|---|---|---|---|---|
| | SEQ | | (1:10 dilution, | |
| | ID | | 5 minutes | MIC with |
| Rank | NO: | Peptide sequence | incubation) | no trypsin |
| 1 | 18 | CWTKSIPPKPCGLRKRLKKIKNKIKEKL | 25 | 25 |
| | | KKIGQKIQGLLPKLAPRTDY | | |
| 2 | 19 | CWTKSIPPKPCGLRKILKKIKNKIKEKL | 50 | 25 |
| | | KKIGQKIQGLLPKLAPRTDY | | |
| 3 | 21 | CWTKSIPPKPCGLRKRLRKIKNKIKEK | 50 | 100 |
| | | LKKIGQKIQGLLPKLAPKTDY | | |
| 4 | 22 | CWTKSIPPKPCGCRKPLRKIRNKIKEKL | 100 | 100 |
| | | KKIGQKIQGLLPKLAPRTDY | | |
| 5 | 20 | CWTKSIPPKPCGLRKKLKKIRNKIKEKL | 100 | 50 |
| | | KKIGQKIQGLLPKLAPRTDY | | |
| 5 | 23 | CWTKSIPPKPCGCRKPCRKIRNKIKEK | >400 | >400 |
| | | LKKIGQKIQGLLPKLAPRTDY | | |
| 6 | 1 | GLRKRLRKFRNKIKEKLKKIGQKIQGL | >400 | 25 |
| | | LPKLAPRTDY | | |

TABLE 8
Experiment 2-1:10 diluted trypsin

| | | | MIC with trypsin | |
|---|---|---|---|---|
| | SEQ | | (1:10 dilution, | |
| | ID | | 5 minutes | MIC with |
| Rank | NO: | Peptide sequence | incubation) | no trypsin |
| 1 | 18 | CWTKSIPPKPCGLRKRLKKIKNKIKEKLKKI | 25 | 25 |
| | | GQKIQGLLPKLAPRTDY | | |
| 2 | 19 | CWTKSIPPKPCGLRKILKKIKNKIKEKLKKIG | 50 | 50 |
| | | QKIQGLLPKLAPRTDY | | |
| 3 | 21 | CWTKSIPPKPCGLRKRLRKIKNKIKEKLKKI | 50 | 50 |
| | | GQKIQGLLPKLAPKTDY | | |
| 4 | 22 | CWTKSIPPKPCGCRKPLRKIRNKIKEKLK | 100 | 100 |
| | | KIGQKIQGLLPKLAPRTDY | | |
| 5 | 20 | CWTKSIPPKPCGLRKKLKKIRNKIKEKLK | 100 | 50 |
| | | KIGQKIQGLLPKLAPRTDY | | |
| 6 | 23 | CWTKSIPPKPCGCRKPCRKIRNKIKEKLK | >400 | >400 |
| | | KIGQKIQGLLPKLAPRTDY | | |
| 7 | 1 | GLRKRLRKFRNKIKEKLKKIGQKIQGLLP | >400 | 25 |
| | | KLAPRTDY | | |

TABLE 9
Experiment 3-1:2 diluted trypsin

| | | | MIC with trypsin | |
|---|---|---|---|---|
| | SEQ | | (1:2 dilution, | |
| | ID | | 5 minutes | MIC with |
| Rank | NO: | Peptide sequence | incubation) | no trypsin |
| 1 | 18 | CWTKSIPPKPCGLRKRLKKIKNKIKEKLK | 12.5 | 12.5 |
| | | KIGQKIQGLLPKLAPRTDY | | |
| 2 | 19 | CWTKSIPPKPCGLRKILKKIKNKIKEKLK | 25 | 12.5 |
| | | KIGQKIQGLLPKLAPRTDY | | |
| 3 | 21 | CWTKSIPPKPCGLRKRLRKIKNKIKEKLK | 25 | 25 |
| | | KIGQKIQGLLPKLAPKTDY | | |
| 4 | 22 | CWTKSIPPKPCGCRKPLRKIRNKIKEKLK | 50 | 25 |
| | | KIGQKIQGLLPKLAPRTDY | | |
| 5 | 20 | CWTKSIPPKPCGLRKKLKKIRNKIKEKLK | 50 | 25 |
| | | KIGQKIQGLLPKLAPRTDY | | |
| 6 | 23 | CWTKSIPPKPCGCRKPCRKIRNKIKEKLK | >400 | 200 |
| | | KIGQKIQGLLPKLAPRTDY | | |
| 7 | 1 | GLRKRLRKFRNKIKEKLKKIGQKIQGLLP | >400 | 12.5 |
| | | KLAPRTDY | | |

TABLE 10
Experiment 4-1:2 diluted trypsin

| | | | MIC with | |
|---|---|---|---|---|
| | | | trypsin | |
| | SEQ | | (1:2 dilution, | |
| | ID | | 5 minutes | MIC with |
| Rank | NO: | Peptide sequence | incubation) | no trypsin |
| 1 | 18 | CWTKSIPPKPCGLRKRLKKIKNKIKEKLK | 12.5 | 12.5 |
| | | KIGQKIQGLLPKLAPRTDY | | |
| 2 | 19 | CWTKSIPPKPCGLRKILKKIKNKIKEKLK | 25 | 25 |
| | | KIGQKIQGLLPKLAPRTDY | | |
| 3 | 21 | CWTKSIPPKPCGLRKRLRKIKNKIKEKLK | 25 | 12.5 |
| | | KIGQKIQGLLPKLAPKTDY | | |
| 4 | 22 | CWTKSIPPKPCGCRKPLRKIRNKIKEKLK | 50 | 25 |
| | | KIGQKIQGLLPKLAPRTDY | | |
| 5 | 20 | CWTKSIPPKPCGLRKKLKKIRNKIKEKLK | 50 | 25 |
| | | KIGQKIQGLLPKLAPRTDY | | |
| 6 | 23 | CWTKSIPPKPCGCRKPCRKIRNKIKEKLK | >400 | 200 |
| | | KIGQKIQGLLPKLAPRTDY | | |
| 7 | 1 | GLRKRLRKFRNKIKEKLKKIGQKIQGLLP | >400 | 12.5 |
| | | KLAPRTDY | | |

TABLE 11
Experiment 5-Undiluted trypsin

| | | | MIC with | |
|---|---|---|---|---|
| | | | trypsin | |
| | | | (Undiluted, | |
| | SEQ | | 5 minutes | MIC with |
| Rank | ID NO: | Peptide sequence | incubation) | no trypsin |
| 1 | 18 | CWTKSIPPKPCGLRKRLKKIKNKIKEKLK | 25 | 12.5 |
| | | KIGQKIQGLLPKLAPRTDY | | |
| 2 | 19 | CWTKSIPPKPCGLRKILKKIKNKIKEKLK | 25 | 25 |
| | | KIGQKIQGLLPKLAPRTDY | | |
| 3 | 22 | CWTKSIPPKPCGCRKPLRKIRNKIKEKLK | 50 | 25 |
| | | KIGQKIQGLLPKLAPRTDY | | |
| 4 | 20 | CWTKSIPPKPCGLRKKLKKIRNKIKEKLK | 50 | 25 |
| | | KIGQKIQGLLPKLAPRTDY | | |
| 5 | 21 | CWTKSIPPKPCGLRKRLRKIKNKIKEKLK | 50 | 12.5 |
| | | KIGQKIQGLLPKLAPKTDY | | |
| 6 | 23 | CWTKSIPPKPCGCRKPCRKIRNKIKEKLK | >400 | 200 |
| | | KIGQKIQGLLPKLAPRTDY | | |
| 7 | 1 | GLRKRLRKFRNKIKEKLKKIGQKIQGLLP | >400 | 12.5 |
| | | KLAPRTDY | | |

Trypsin from Sigma-Aldrich, 2.5 mg/ml Stock Concentration

TABLE 12
Experiment 6-Undiluted trypsin

| | | | MIC with | |
|---|---|---|---|---|
| | | | trypsin | |
| | | | (Undiluted, | |
| | SEQ | | 5 minutes | MIC with |
| Rank | ID NO: | Peptide sequence | incubation) | no trypsin |
| 1 | 18 | CWTKSIPPKPCGLRKRLKKIKNKIKEKLK | 25 | 12.5 |
| | | KIGQKIQGLLPKLAPRTDY | | |
| 2 | 19 | CWTKSIPPKPCGLRKILKKIKNKIKEKLK | 25 | 25 |
| | | KIGQKIQGLLPKLAPRTDY | | |
| 3 | 22 | CWTKSIPPKPCGCRKPLRKIRNKIKEKLK | 50 | 25 |
| | | KIGQKIQGLLPKLAPRTDY | | |
| 4 | 20 | CWTKSIPPKPCGLRKKLKKIRNKIKEKLK | 50 | 25 |
| | | KIGQKIQGLLPKLAPRTDY | | |
| 5 | 21 | CWTKSIPPKPCGLRKRLRKIKNKIKEKLK | 50 | 25 |
| | | KIGQKIQGLLPKLAPKTDY | | |
| 6 | 23 | CWTKSIPPKPCGCRKPCRKIRNKIKEKLK | >400 | 200 |
| | | KIGQKIQGLLPKLAPRTDY | | |
| 7 | 1 | GLRKRLRKFRNKIKEKLKKIGQKIQGLLP | >400 | 12.5 |
| | | KLAPRTDY | | |

Trypsin from Sigma-Aldrich, 2.5 mg/ml Stock Concentration

TABLE 13
Undiluted and diluted trypsin compared to without trypsin

| | | | | | | |
|---|---|---|---|---|---|---|
| Trypsin | | | | | | |
| CAP18_WT | Undiluted | 2-fold | 4-fold | 8-fold | 16-fold | 32-fold |
| cap18_hphobic2_NTinh | Undiluted | 2-fold | 4-fold | 8-fold | 16-fold | 32-fold |
| cap18_CRKP1_NTinh | Undiluted | 2-fold | 4-fold | 8-fold | 16-fold | 32-fold |
| cap18_CRKP2_NTinh | Undiluted | 2-fold | 4-fold | 8-fold | 16-fold | 32-fold |
| cap18_R2Kv2_NTinh | Undiluted | 2-fold | 4-fold | 8-fold | 16-fold | 32-fold |
| cap18_R2Kv3_NTinh | Undiluted | 2-fold | 4-fold | 8-fold | 16-fold | 32-fold |
| cap18_R2Kv4_NTinh | Undiluted | 2-fold | 4-fold | 8-fold | 16-fold | 32-fold |
| No trypsin | | | | | | |
| CAP18_WT | Undiluted | 2-fold | 4-fold | 8-fold | 16-fold | 32-fold |
| cap18_hphobic2_NTinh | Undiluted | 2-fold | 4-fold | 8-fold | 16-fold | 32-fold |
| cap18_CRKP1_NTinh | Undiluted | 2-fold | 4-fold | 8-fold | 16-fold | 32-fold |
| cap18_CRKP2_NTinh | Undiluted | 2-fold | 4-fold | 8-fold | 16-fold | 32-fold |
| cap18_R2Kv2_NTinh | Undiluted | 2-fold | 4-fold | 8-fold | 16-fold | 32-fold |
| cap18_R2Kv3_NTinh | Undiluted | 2-fold | 4-fold | 8-fold | 16-fold | 32-fold |
| cap18_R2Kv4_NTinh | Undiluted | 2-fold | 4-fold | 8-fold | 16-fold | 32-fold |

Trypsin from Sigma-Aldrich, 2.5 mg/ml Stock Concentration

### Example 4

### Evaluating the Therapeutic Effect of CAP18 Peptides Against Bovine Respiratory Disease (BRD) in Cattle

Native Lactobacillus, Bacillus, or other commensal strains isolated from bovine upper respiratory tract, Lactobacillus reuteri ATCC PTA-126788 and/or Bacillus subtilis ATCC PTA-126786 are chromosomally engineered to deliver CAP18 and BMAP28. The engineered strains delivering CAP18 and BMAP28 are confirmed for expression, secretion, functionality, and stability and evaluated for efficacy in an experimental BRD model along with naked CAP18 and BMAP peptides. In this model, on day 0, infectious Bovine Rhinotracheitis (IBR) and Bovine Viral Diarrhoea Virus 1b (BVDV-1b) Viral Seeder cattle are comingled with contact animals. On the same day, Investigational Veterinary Products (IVPs) containing vector strains delivering CAP18 peptide(s) are administered intranasally at a dose of 1×10⁸ CFUs/cattle. On day 4, the Viral Seeder cattle are inoculated with Mannheimia haemolytica. Peak BRD clinical signs occur between day 4 and day 12 and the animals are necropsied on day 24. A BRD case is defined as cattle with fever 2104° F., Depression Score of 21 or Respiratory Characterization Score of 21. Peak mortality occurs between 8 to 14 days. Day 12 to day 24 serves as the chronic BRD phase. In this model, each animal is considered as the experimental unit. Percent lung lesion is the primary variable. Achievement of BRD case definition is secondary variable. Tulathromycin is used as a positive control. With tulathromycin treatment, lung consolidation generally reduces from approximately 30% to 5%.

### Example 5

### Evaluating the Therapeutic Effect of CAP18 Peptides Against Coccidiosis in Poultry

Lactobacillus reuteri ATCC PTA-126788 or Bacillus subtilis ATCC PTA-126786 bacteria delivering CAP18 peptides is administered to embryonated eggs on day 18 or as spray on day of hatch. A second dose of Lactobacillus or Bacillus is administered in drinking water on day 8. One hundred twenty day-old chicks are randomly allocated into 4 groups with 30 chicks in each group. Group I is administered with Lactobacillus in ovo to day 18 embryonated eggs or as spray on day of hatch and a second dose of L. reuteri is administered in drinking water on day 8 after hatching. Bacillus will be administered in feed every day. On day 10, all the birds in group I are orally gavaged with 15,000 oocysts containing 5,000 oocysts of Eimira maxima, 5,000 oocysts of Eimeria tenella and 5000 oocysts of Eimeria acervulina. Group II serves as a positive control and is orally gavaged with 15,000 oocysts containing 5,000 oocysts of Eimeria maxima, 5,000 oocysts of Eimeria tenella and 5,000 oocysts of Eimeria acervulina and treatment with a coccidiostat. Group III serves as a challenge control and is orally gavaged with 15,000 oocysts containing 5,000 oocysts of Eimeria maxima, 5,000 oocysts of Eimeria tenella and 5000 oocysts of Eimeria acervulina. Group IV serves as no challenge control. Droppings from each treatment group are collected and assessed for oocyst shedding at the peak of cycling, 2-3 days later as well as at the end of the study. 4 birds from each treatment group are necropsied at the peak of cycling and intestines are scored for Coccidiosis-specific lesions. Feed intake and body weights are also recorded at the end of the study.

### Example 6

### Therapeutic Effect of CAP18 Against Giardia in Dogs

Twelve dogs are randomly assigned into 4 groups with 3 dogs in each group. Groups I, II and III are orally gavaged with 1.35×10⁶ trophozoites or cysts of Giardia. Group I is orally gavaged with Lactobacillus reuteri ATCC PTA-126788 or Bacillus subtilis ATCC PTA-126786 delivering CAP18 on study days—7, 0, 7, 14, 28, 56, 112. Group II serves as a positive control and the dogs will be treated with metronidazole. Group III serves as challenge control and will not receive Lactobacillus treatment. Group IV serves as no challenge control and will not receive Giardia challenge, Lactobacillus treatment or metronidazole treatment. All the dogs are monitored for clinical signs every day in the first 2 weeks and then on a weekly basis until the end of study on day 124. Two days before inoculation, on the day of inoculation and every 7 days after inoculation with Giardia, feces are collected and monitored for Giardia oocysts by floatation method. Five ml blood samples are collected from each dog before the inoculation and at the end of the study and analyzed for blood chemistry.

### Example 7

As shown in FIGS. 10A and 106, in-vitro screening of the antimicrobial peptide CAP18, which is referred to as AMP-1 in FIGS. 10A and 108, shows that CAP18 inhibits the growth of the rumen methanogen, M. byranti. BES, a known methanogen inhibitor shown in FIG. 10C, was used as a positive control.

### Example 8

### Media

M. ruminantium, M. bryantii, and M. gottschalkii are grown on DSM 119 media with H₂:CO₂ (80:20) as substrate. The media consisted of the following composition: 3.67 mM KH₂PO₄, 1.62 mM MgSO₄×7H₂O, 6.84 mM NaC, 7.48 mM NH₄Cl, 0.34 mM CaCl₂)×2H₂O, 7.18 μM FeSO₄×7H₂O, Trace element solution SL-10, 3.65 mM yeast extract, 12.2 mM Na-acetate, 29.41 mM Na-formate, 30 m/L rumen fluid, fatty acid mixture, resazurin, 47.62 mM NaHCO₃, 2 mM L-cysteine-HCl×H2O, and 2 mM Na₂S×9H₂O. The media was prepared with boiling method. Supplementation of 3.52 mM coenzyme-M is added from a filter-sterilized stock for the growth of M. ruminantium.

### AMP Preparations

Freeze-dried AMP powder were dissolved in nuclease-free water at 120 μM concentration and aliquoted to a sterile aluminum-covered Wheaton bottle. The solution was made anaerobic with 10 psi N₂ and stored anaerobically at −20° C.

Modified protocol: For the titration of AMPs, reduced DSM119 media was used as a diluent instead of water. The AMP solutions were then aliquoted to 5 ml solution right before use to prevent freeze-thawing.

### AMP Screening

Methanogens are grown in a 5 ml media with its respective substrate and placed in a shaker operated at 37° C., 100 rpm. Four sampling of 200 μl was taken prior to the AMP addition at 12 hour apart until the end of the lag phase as measured by spectrophotometer. When the growth reached mid—or late-log, corresponding to OD₆₀₀ₙₘ of 0.3-0.4, 100 μl of each AMP solution was added anaerobically. The effect of AMP addition on growth was observed for 12-h after the addition, with sampling every 2-3 hour. FIGS. 11A-G show the anti-methanogenic activities of antimicrobial peptides CAP18, BMAP-28, K9 cathelicidin, BAC-7, CAP18 variant, LL-37, PMAP-23, respectively, against rumen Methanogen, Methanobrevibacter ruminantium. FIGS. 12A-E show the anti-methanogenic activities of antimicrobial peptides CAP18, CAP18 variant, Bac-7, BMAP-28, LL-37, respectively, against rumen Methanogen, Methanobacter bryantii.

### AMP Sequences

The following antimicrobial peptides were used in this experiment having the indicated sequences:

>CAP18 (AMP1)
(SEQ ID NO: 1)
GLRKRLRKFRNKIKEKLKKIGQKIQGLLPKLAPRTDY;
>CAP18_variant (R2Kv3_NTinh) (AMP 4)
(SEQ ID NO: 18)
CWTKSIPPKPCGLRKRLKKIKNKIKEKLKKIGQKIQGLLPKLAPRTDY;
>BMAP-28 WT (AMP 3)
(SEQ ID NO: 50)
GGLRSLGRKILRAWKKYGPIIVPIIRIG;
>Bac7 WT (AMP 2)
(SEQ ID NO: 63)
RRIRPRPPRLPRPRPRPLPFPRPGPRPIPRPLPFPRPGPRPIPRPLPFP
RPGPRPIPRPL;
k9Cath WT (AMP 5)
(SEQ ID NO: 77)
RLKELITTGGQKIGEKIRRIGQRIKDFFKNLQPREEKS;
>LL-37 WT (AMP 6)
(SEQ ID NO: 118)
LLGDFFRKSKEKIGKEFKRIVQRIKDFLRNLVPRTES;
>PMAP23 WT (AMP 7)
(SEQ ID NO: 119)
RIIDLLWRVRRPQKPKFVTVWVR.

### Example 9

### Evaluating the Therapeutic Effect of CAP18 Peptides Against Coccidiosis in Chickens

The objective of this study is to evaluate the safety and efficacy of native Lactobacillus reuteri ATCC PTA-126788 or Bacillus subtilis ATCC PTA-126786 bacteria engineered to deliver CAP18 and BMAP28 polypeptides of this disclosure in broiler chickens when administered orally against an Eimeria maxima (E. maxima) challenge. In place of these, alternative peptides disclosed and described herein can be utilized and evaluated. Eimeria maxima is one of the three primary coccidia affecting broilers throughout the poultry industry. It has been demonstrated to be the most frequent cause of Intestinal epithelium damage that initiates conditions favorable for Clostridium perfringens to proliferate and produce toxin resulting in Necrotic Enteritis. Enrollment will include 300 chicks (plus 10 extra birds which will be used for serum baseline). There will be 6 replicates of 10 chicks (60 chicks in total) enrolled into each Treatment Group. The IVPs will be administered to 1-day-old chicks via oral gavage and a second administration on SD 7. In this first study a target dose of 1×10⁸ CFU/bird will be administered.

### Study Design-Treatment Groups

TABLE 14
Treatment Groups

| | | Number of | | | | | |
|---|---|---|---|---|---|---|---|
| Treatment | Number of | Chicks per | IVP | IVP Target | Route of | Day of | Challenge |
| Group | Replicates | Replicate | Treatment | Dose | Treatment | Treatment | E. maxima |
| 1 | 6 | 10 | None | N/A | N/A | N/A | N/A |
| | | | (untreated | | | | |
| | | | unchallenged | | | | |
| | | | control) | | | | |
| 2 | 6 | 10 | None | N/A | N/A | N/A | SD 14 |
| | | | (untreated | | | | |
| | | | challenged | | | | |
| | | | control) | | | | |
| 3 | 6 | 10 | Amprolium | 113.5 | N/A | Starter | SD 14 |
| | | | (positive | grams/US | | Mash | |
| | | | control) | ton | | (SD 0-28) | |
| 4 | 6 | 10 | Bacteria | 1 × 10⁸ | Oral | SD 0; | SD 14 |
| | | | delivering | CFUs/ | gavage | SD 7 | |
| | | | CAP18 | bird/day | | | |
| | | | peptides | | | | |
| 5 | 6 | 10 | Bacteria | 1 × 10⁸ | Oral | SD 0; | SD 14 |
| | | | delivering | CFUs/ | gavage | SD 7 | |
| | | | BMAP28 | bird/day | | | |
| | | | peptides | | | | |

Abbreviations: SD = Study Day; N/A = Not applicable.

### Study Procedures

This study will utilize three hundred (300), day-old Ross×Ross broiler chicks; 10 additional birds will be used for serum baseline. On SD 0, 60 chicks from each Treatment Group (1, 2, 3, 4 and 5) will be randomly assigned to 6 replicates per Treatment Group at 10 chicks per replicate using randomized complete block. Chicks from each replicate will be housed in the same cage. On SD 0, all chicks in Treatment Groups 4 and 5 will receive 1×10⁸ CFU/0.2 mL dose of Lactobacillus or Bacillus, CAP18 or BMAP28 delivering IVPs, respectively, via oral gavage. On SD 7, all chicks in Treatment Groups 4 and 5 will receive 1×10⁸ CFU/0.2 mL dose of Lactobacillus or Bacillus, CAP18 or BMAP28 delivering IVPs, respectively, via oral gavage. On SD 0-28, Treatment Group 3 will receive Amprolium in the feed at 113.5 grams per ton of feed. On SD 13, if there are more than 2 mortalities in any replicate, chicks will be replaced with chicks from replicates within the same Treatment Group to ensure that there are equal numbers of chicks, as far as possible, in each replicate within the Treatment Group. On SD 14, all chicks in Treatment Groups 2, 3, 4 and 5 will be challenged with about 25000 oocysts/ml of E. maxima (dose will be documented in the FSR) via oral gavage as described by Chapman et al. 2005. On SD 20, 4 chicks from each replicate (total of 24 chicks from each Treatment Group) will be randomly selected, euthanized, necropsied, and coccidia lesion scored. Cecal samples for microbiome profiling, histopathology and transcriptomics from lesion score birds will be collected. Feces from each cage for OPG will be collected. On SD 23, SD 26 and SD 28, feces from each cage for OPG will be collected. On SD 28, the number of chicks alive in each cage will be recorded then all chickens will be removed from each cage and euthanized. 5 birds per cage will be bled for serology and samples for microbiome, histology and transcriptomics from two (2) birds/cage will be collected.

### Schedule of Events

TABLE 15
Schedule of Events

| Study Day | |
|---|---|
| (SD) | Event |
| SD 0 | Pick up chicks from hatchery |
| | Place birds into Isolation Room 6 |
| | Chicks allocated to cages (30 cages with 10 birds/cage) |
| | Bleed ten (10) extra birds and store serum for baseline |
| | Treat birds (oral gavage) with IVP |
| | Start administering Amprolium in the feed of Treatment |
| | Group 3 at |
| | 113.5 grams per ton of feed (to be continued until SD 28) |
| SD 7 | Boost Treat birds (oral gavage) |
| SD 14 | Inoculate all birds with ~25,000 oocysts of E. maxima |
| SD 20 | Bleed lesion score birds for serology |
| | Lesion score four (4) birds/cage by Johnson and Reid Scoring |
| | System Collect samples for microbiome, histology and |
| | transcriptomics from lesion score birds |
| | Collect feces from each cage for OPG |
| SD 23 | Collect feces from each cage for OPG |
| SD 26 | Collect feces from each cage for OPG |
| SD:28 | Collect feces from each cage for OPG |
| | Bleed five (5) birds per cage for serology |
| | Collect samples for microbiome, histology and |
| | transcriptomics from two (2) birds/cage |
| | Terminate trial |

The experimental unit will be the cage. Primary variable is E. maxima lesion score on SD 20.
The secondary variable will be OPG on SD 20, 23, 26, 28.

### Animal Management

### Housing and Husbandry

At the study site, chicks will be housed in an animal house facility with lighting that may be provided on an 18-hour light and 6 hours darkness. Temperature will be maintained and adjusted appropriately to temperatures that are suitable for the age of the chicks. Housing will consist of 2 racks that contain 5 rows of cages at 3 columns per row, totaling 15 cages per rack. Each cage is approximately 27″×27″ allowing for approximately 5.1 square feet of spacing (stocking density of 0.63 square feet per bird). Each housing cage will be checked at least twice daily, in accordance with the study site standard operating procedures (SOPs). Feed and water will be available ad libitum throughout the trial. Each cage will contain 1 (one) trough feeder and 1 (one) trough drinker (10 bird to feeder/drinker ratio, 24 inch×3.5 inch trough). All the feeders and waterers will be checked at least twice daily during regular health visits.

Additional feed and water will be added as needed.

### Animal Disposition

Following study initiation, all dead or euthanized chicks will be necropsied to determine cause of death or removal then properly disposed of. At the end of the study, all remaining chickens will be euthanized with CO₂ or via cervical dislocation, in accordance with current American Veterinary Medical Association Guidelines, and disposed of according to site procedures and permit requirement.

### Randomization Procedures

Treatment Groups will be blocked within the two cage racks so that each of the 5 rows of cages contains one Treatment Group (3 columns per row, 2 racks, totaling 6 replicates) starting with the untreated unchallenged control (Treatment Group 1) on the top of the rack to avoid contamination with Eimeria maxima oocysts.

### Feed Formulation and Storage

Food rations will be a commercial-type broiler diet and will consist of non-medicated feed that is free of probiotic (except for Treatment group 3 Amprolium will be added). The feed formulations are as follows.

TABLE 16

| Ingredient | Wt % |
|---|---|
| Corn | 58.625 |
| Soybean meal | 34.969 |
| Vegetable oil | 2.607 |
| Dicalcium phosphate | 1.505 |
| Calcium carbonate | 0.88 |
| MHA | 0.384 |
| NaCl | 0.328 |
| L-Lysine | 0.266 |
| Trace mineral premix | 0.1 |
| Sodium bicarbonate | 0.094 |
| L-Threonine | 0.088 |
| Choline chloride (60%) | 0.068 |
| Vitamin premix | 0.05 |
| L-Valine | 0.026 |
| Phytase (500 ftu) | 0.01 |

### Investigational Veterinary Product Preparation and Administration

On SD 0 and SD 7 respectively, each vial will be diluted with the appropriate volume of distilled water or equivalent. The IVPs and control are administered by oral gavage.

### Investigational Veterinary Product Titration

Immediately following IVP resuspension at each administration timepoint, 100 μL will be taken and used to perform 10-fold serial dilutions, up to 10⁻⁶. Remove 100 μL from the undiluted aliquot and 100 μL from each of the serial dilutions (undiluted, 10⁻¹, 10⁻², 10⁻³, 10⁻⁴, 10⁻⁵, and 10⁻⁶) and plate each onto separate Trypticase Soy Agar (TSA) agar plates in duplicates. Incubate plates under aerobic conditions at 37° C. for up to 24 hours. Following incubation, remove plates from incubator, determine number of CFU/mL

### Challenge Strain

E. maxima Challenge Preparation and Administration

E. maxima challenge inoculum will be stored between 2-8° C. until time of challenge. On SD 14, chickens in Treatment Groups 2, 3, 4, and 5 will be challenged with sporulated oocysts of E. maxima (^{˜}25,000 oocysts). The challenged inoculum will be administered via oral gavage (1 ml/bird) using a 10 ml syringe fitted with an 18-gauge feeding/gavage needle.

E. maxima Lesion Scoring

On SD 20, four birds from each replicate (24 total from each Treatment Group) will be euthanized and examined for E. maxima lesions. The E. maxima lesion will be scored on a 0 to 4 scale. Based on the scoring scale, 0=no gross lesions, 1=small petechiae at serosal side of mid-intestine, no ballooning or thickening of the intestine, small amounts of orange mucus, 2=serosal surface with numerous red petechiae, intestine may be filled with orange mucus, little or no ballooning of the intestine, thickening of the wall, 3=intestinal wall ballooned and thickened, mucosal surface roughened; intestinal contents filled with pinpoint blood clots and mucus, 4=intestinal wall ballooned for most of its length; contains numerous blood clots and digested red blood cells giving a characteristic color and putrid odor; the wall is greatly thickened; dead birds are recorded with this score.

### Sample Collection and Analysis

### Oocysts Per Gram (OPG) of Feces

On days 20, 23, 26 and 28, a fecal sample will be collected from each cage for OPG counts.

### Example 10

Evaluation of an Intranasal CAP18 and BMAP28 peptides in Suspension in a Natural BRD Challenge Model Utilizing Viral and Bacterial Seeders in Conjunction with Environmental and Husbandry Stressors

### Objective

The objective is to evaluate the efficacy of an intranasal native Lactobacillus, Bacillus or other commensal strains isolated from bovine upper respiratory tract, Lactobacillus reuteri ATCC PTA-126788 or Bacillus subtilis ATCC PTA-126786, which are chromosomally engineered to deliver CAP18 and BMAP28 polypeptides of this disclosure, in a natural BRD challenge model utilizing viral and bacterial seeders in conjunction with environmental and husbandry stressors. In place of these, alternative peptides disclosed and described herein can be utilized and evaluated. The engineered strains delivering CAP18 and BMAP28 peptides of this disclosure are confirmed for expression, secretion, functionality, and stability and evaluated for efficacy in this experimental BRD model.

No in vitro mechanism is available to mimic the diverse viral, bacterial, genetic, and environmental/husbandry interactions that support and promote a BRD episode. As such, a host animal disease model is the most appropriate mechanism for mimicking as well as evaluating candidates to control/reduce BRD.

### Study Design

TABLE 17
Treatment Group (TG) and Seeder Group (SG) Details
TG Details

| | | | | IVP/ | | | | | |
|---|---|---|---|---|---|---|---|---|---|
| TG | TG | Dosing | Dose | CP Con. | IVP/CP | Dose | Dose | SD of | |
| ID | Name | Compound | (mg/kg) | (mg/mL) | Route | Volume | Location | Dosing | n |
| 1 | Negative | Saline | NA | NA | IN | 6 mL per | Bilateral | 0-4 | 11 |
| | Control | | | | | nostril | Nasal | | |
| 2 | Positive | Draxxin ™ | 2.5 | 100 | SQ | by BW | Left | 0 | 9 |
| | Control | (tulathromycin) | | | | | Neck | | |
| 3 | Bacteria | Bacteria | NA | 1 | IN | 6 mL per | Bilateral | 0-4 | 18 |
| | Delivering | delivering | | | | nostril | Nasal | | |
| | CAP18 | CAP18 | | | | | | | |
| | peptides | peptides | | | | | | | |
| 4 | Bacteria | Bacteria | NA | 1 | IN | 6 mL per | Bilateral | 0-4 | 18 |
| | delivering | delivering | | | | nostril | Nasal | | |
| | BMAP28 | BMAP28 | | | | | | | |
| | peptides | peptides | | | | | | | |

| | |
|---|---|
| Total TG Calves | 74 |

SG Details

| SG | | | |
|---|---|---|---|
| ID | SG Name | Challenge Details | N |
| A | IBR Seeders with Mh | All Seeder calves inoculated with IBR according to Table | 13 |
| | | 19 on SD 0 | |
| | | All Seeder calves inoculated with Mh according to Table | |
| | | 21 on SD 4 | |
| B | BVDV Seeders with Mh | All Seeder calves inoculated with BVDV1b according to | 13 |
| | | Table 20 on SD 0 | |
| | | All Seeder calves inoculated with Mh according to Table | |
| | | 21 on SD 4 | |

| | |
|---|---|
| Total SG Calves | 26 |
| Total Study Calves | 100 |

Notes:
All SG calves will be individually challenged via a MAD Intranasal ™ syringe tip atomizer or the MAD Teleflex ™ bottle atomizer. A single atomizer can be used for each SG (use a new atomizer for each SG).

TABLE 18
Sequence of Critical Events

| SD | Event |
|---|---|
| −7 | 74 conventional Contact and 26 colostrum-deprived Seeder calves arrive and penned |
| | separately without nose-to-nose contact in the Large Animal Challenge (LAC) facility |
| | Ventilation in the animal room will be maintained at approximately 14 air exchanges/hour and |
| | at a temperature of 60-70° F. during acclimation (from arrival until SD 0) will be maintained |
| −6 to −3 | Acclimation |
| | No clinical activities |
| −2 | Collect clinical observations and rectal temperatures on all (Contacts and Seeders) cattle in the |
| | AM |
| | Collect body weights on only the Contacts and report to the Statistician for randomization |
| −1 | Collect clinical observations and rectal temperatures on all (Contacts and Seeders) cattle in the |
| | AM |
| | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| | Collect 1 pharyngeal swab from all Contacts |
| | Collect 1 nasal swab from all Contacts |
| | Collect 1 × 10 mL SST from all Contacts |
| | Administer IVP/CP to all TGs (1-4) according to Table 17 |
| | Inoculate Seeders with IBR and BVDV1b according to Table 17, 19 and commingle with the |
| | Contacts following inoculation |
| | Initiate bedding and ventilation practices |
| 0-5 | Reduce the floor space by half for approximately 6-8 hours a day until the PM on SD 6 |
| | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| | Administer IVP/CP to TGs 1, 3, 4 according to Table 17 |
| 2 | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| | Administer IVP/CP to TGs 1, 3, 4 according to Table 17 |
| | Collect 1 nasal swab from all Contacts |
| 3 | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| | Administer IVP/CP to TGs 1, 3, 4 according to Table 17 |
| | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| | Administer IVP/CP to TGs 1, 3, 4 according to Table 17 |
| | Inoculate Seeder Groups A & B according to Table 17 & 21 |
| 5 | Collect clinical observations and rectal temperatures on all cattle (Contact and Seeders) in the |
| | AM with clinical observations and rectal temperatures collected in the PM only on Contact |
| | calves that did not meet the BRD protocol definition in the AM. |
| | Collect 1 pharyngeal swab from all Contacts |
| | Collect 1 nasal swab from all Contacts |
| 6 | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| | Discontinue reducing the floor space in the PM |
| 7-9 | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| 10 | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| | Collect 1 pharyngeal swab from all Contacts |
| | Collect 1 nasal swab from all Contacts |
| 11 | Collect clinical observations and rectal temperatures on all cattle (Contact and Seeders) in the |
| | AM with clinical observations and rectal temperatures collected in the PM only on Contact |
| | calves that did not meet the BRD protocol definition in the AM |
| 12 | Collect clinical observations and rectal temperatures on all cattle (Contacts) in the AM with |
| | clinical observations and rectal temperatures collected in the PM only on Contact calves that |
| | did not meet the BRD protocol definition in the AM |
| 13 | Collect clinical observations and rectal temperatures on all Contacts in the AM only |
| 14 | All remaining/surviving Seeder calves removed, euthanized, and lungs scored |
| 14-23 | Collect clinical observations and rectal temperatures on all Contacts in the AM only |
| 24 | Collect clinical observations and rectal temperatures on all Contacts in the AM only |
| | Collect 1 × 10 mL SST from all Contacts |
| | Euthanize remaining/surviving Contacts Score lung lesions Collect |
| | post mortem samples according to Table 12 |
| | In-life completed |

### Experimental Design

This is a non-GXP proof of concept study in the host animal evaluating the ability of an intranasal Lactobacillus reuteri ATCC PTA-126788 or Bacillus subtilis ATCC PTA-126786 engineered to deliver CAP18 peptides and BMAP28 peptides to reduce the clinical signs and lung lesions associated with a BRD episode. This study will be controlled, randomized, and blinded. Injectable tulathromycin was used as a positive control and intranasal saline as a negative control. A BRD disease model utilizing Seeder calves inoculated with IBR, BVDV1b and Mh in conjunction with environmental and husbandry stressors will provide the disease pressure for the Contact calves.

### Experimental Unit

### Individual Calf.

### Randomization Procedures

74 Contact Calves: Calf IDs with body weights will be provided to the statistician for randomization into five TGs with 9 calves each in TG 1 and 2 and 18 calves each in TG 3, 4.
Body weights will be collected on SD-2 and 24 for all Contact calves. Weight will be collected in kilograms (kg).
26 Seeder Calves: The statistician will be provided with calf IDs, but not body weights for the Seeder calves for randomization. Seeder calves will be placed into 4 SGs with 7 calves in each group.
Note: All 26 seeder calves will be of the same immune status (CD calves that are serologically negative to IBR and BVDV and screened at ISUVDL prior to calf delivery) allowing any of the 26 calves to be assigned to any of the SGs (A or B).

### Blinding Method

Blinding will be accomplished through separation of function. Individuals administering the IVP/CP will not be performing clinical observations or laboratory activities. Individuals administering the challenge material to the SGs do not need to be blinded.

### Cattle Inclusion Criteria and Identification

All calves (Contacts and Seeders) in the study will be persistently infected (PI) negative to BVDV and will not have been administered any antibiotics within 14 days of SD-7.

74 Contact Calves: Calves will be eligible if they are non-CD and have no history of being administered any anti-viral/bacterial BRD (IBR, BVDV, BRSV, PI₃, and common BRD bacterial pathogens) vaccines/health products/antibiotics.
Note: Contact calves are allowed to be vaccinated with a product with efficacy against Mycoplasma bovis as the disease model being utilized in this study was developed with calves with this vaccination status.
26 Seeder Caes: Calves will be eligible if they are CD and demonstrate serological negativity to IBR and BVDV via a reputable diagnostic lab assay.
Note: All CD calves will be screened and will be serologically negative for both IBR and BVDV prior to calf arrival and will be appropriate for placement into any SG from A or B.
Acclimation Phase Length All calves will have a S day minimum acclimation period.

### IBR, BVDV and Mh Challenge Details

Tables 19-21 describes the inoculation material and procedure for administration.

TABLE 19
IBR Inoculation Material and Procedure

| | |
|---|---|
| Name of Challenge: | IBR (Cooper Strain) |
| SGs Inoculated: | A |
| Total Calves Inoculated: | 13 |
| Appearance: | Red to pink liquid |
| Challenge Route: | Intranasal (bilateral) |
| Challenge Mechanism: | Atomization via MAD Intranasal ™ syringe tip atomizer or the MAD |
| | Teleflex ™ bottle atomizer (30-100 micron spray) |
| Target Concentration of | 7.0 Log₁₀ TCID₅₀/mL |
| Inoculation Material: | |
| Dose Volume: | 4 ml total volume administered (approximately 2 mL/nostril) to |
| | each calf |
| Challenge Prep: | Each frozen vial will be thawed and pooled. The pooled material will |
| | be packaged into a plastic vaccine style bottle with a rubber top for |
| | transport to the field for use. |
| Transport Conditions to | Keep chilled on ice/ice packs after preparation on day of challenge |
| Animal Facility: | for transport to the field for use. |
| Inoculation Procedure: | Each calf will be restrained in a head catch and may be haltered with |
| | the nose slightly elevated. A small bag will be placed over the nose |
| | of the calf to hyperventilate. At the first signs of respiratory distress |
| | the bag will be removed and an atomizer will be used to administer |
| | approximately 2 mL of the challenge material into each nostril. |
| Material Titrations: | Both pre- and post-challenge material titrations will be performed. |

TABLE 20
BVDV1b Inoculation Material and Procedure

| | |
|---|---|
| SGs Inoculated: | B |
| Total Calves Inoculated: | 13 |
| Appearance: | Reddish-Pink Liquid |
| Challenge Route: | Intranasal via atomization |
| Challenge Mechanism: | Atomization via MAD Intranasal ™ syringe tip atomizer or the MAD |
| | Teleflex ™ bottle atomizer (30-100 micron spray) |
| Target Concentration of | 5.0 Log₁₀ TCID₅₀/mL |
| Inoculation Material: | |
| Dose volume: | 4 mL total volume administered (approximately 2 mL/nostril) |
| Challenge Prep: | Each frozen vial will be thawed and pooled. The pooled material |
| | will be packaged into a plastic vaccine style bottle with a rubber top |
| | for transport to the field for use. |
| Transport Conditions to | Keep chilled on ice packs after preparation is made on the day of |
| Animal Facility: | challenge for transport to the animal facility |
| Inoculation Procedure: | Each calf will be restrained in a head catch and may be haltered with |
| | the nose slightly elevated. A small bag will be placed over the nose |
| | of the calf to hyperventilate. At the first signs of respiratory distress |
| | the bag will be removed and an atomizer will be used to administer |
| | approximately 2 mL of the challenge material into each nostril. |
| Challenge Material | Both pre- and post-challenge material titrations will be performed. |
| Titrations: | |

TABLE 21
Mh Inoculation Material and Procedure

| | |
|---|---|
| Name of Challenge: | Mh Challenge Culture |
| SGs Inoculated: | A & B |
| Total Calves Inoculated: | 13 |
| Appearance: | Clear with mild turbidity |
| Challenge Route: | Intranasal via atomization |
| Target Concentration of | 1 × 10⁸ per mL |
| Inoculation Material: | |
| Dose volume: | 2 mL of culture will be added to 8 mL of warm phosphate buffered |
| | saline for atomization. Dose volume is 10 mL (approximately 5 |
| | mL/nostril) |
| Packaging: | Culture will be placed into a plastic serum/vaccination container. |
| | Warm phosphate buffered saline will be placed into a plastic |
| | serum/vaccination bottle in individual 8 mL aliquots. At the animal |
| | facility, 2 mL of culture will be removed and added to an 8 mL |
| | aliquot of phosphate buffered saline and mixed for atomization |
| Storage conditions: | Culture will be transported on cold packs and phosphate buffered |
| | saline aliquots will be transported on warm packs to the animal |
| | facility |
| Inoculation Procedure: | The calf will be restrained in a head catch and may be haltered with |
| | the head slightly elevated. A small bag will be placed over the nose |
| | of the animal to hyperventilate the calf. At the first signs of |
| | respiratory distress, the bag is removed and the atomizer will be |
| | used to administer approximately 5 mL of the challenge material |
| | into each nostril via atomization. Following challenge the bag will |
| | be placed over the nose again to hyperventilate the animal to |
| | complete the procedure |
| Inoculation Material | Both pre- and post-challenge material titrations will be performed. |
| Titrations: | |

### Clinical Observations

Temperatures and clinical observations will be collected by a veterinarian or trained designee as described in Table 17 and 22.

TABLE 22
Clinical Observations

| Observation | SD |
|---|---|
| Mortality | See |
| N = not dead | Footnote |
| Y = found dead | Below |
| M = moribund and euthanized | |
| Rectal Temperature | |
| Respiratory Character (nasal discharge, coughing or dyspnea/tachypnea) | |
| 0 = all of the above signs are normal/absent | |
| 1 = one of the above clinical signs are present | |
| 2 = two of the above clinical signs are present | |
| 3 = all three of the above clinical signs are present | |
| Depression | |
| 0 = No depression | |
| 1 = Slight depression; slight disinterest in environment/pen mates and moves | |
| around pen without stimulation | |
| 2 = Moderate depression; moderate disinterest in environment/pen mates | |
| and needs stimulation to move around pen | |
| 3 = Severe depression; severe disinterest in environment/pen mates and | |
| needs stimulation to rise or is recumbent | |

Note:
Rectal Temperature and Clinical Observations for Seeders and Contacts Seeders will have temperatures and clinical observations collected once daily in the AM from SD −2 through SD 11. All surviving Seeders will be euthanized and lung scored on SD 11.
Contacts will have temperatures and clinical observations collected on SD −2 and −1 only in the AM. From SD 0 until SD 12 temperatures and clinical observations will be collected in the AM with clinical observations and rectal temperatures also collected in the PM only on Contact calves that did not meet the BRD protocol definition in the AM. From SD 13 until SD 24 temperatures and clinical observations will only be collected in the AM.

### Sample Collection, Processing and Testing

Table 23 provides details on sample collection.

TABLE 23
Sample Collection, Processing, Storage and Shipment Details

| | | | | | Sample |
|---|---|---|---|---|---|
| Sample | TGs | Sample Collection Details | No. | SD | Use |
| Pharyngeal | 1 & 2 | B = collected with a double guarded | 1 per | 0, 5, 10 | B = meta- |
| Swab A | (only 4 | culture swab and placed into 1 mL DNA | calf | | genomics |
| | calves/TG | Shield placed into an appropriate | (B) | | |
| | | container. | | | |
| | | Note: On SDs with both pharyngeal and | | | |
| | | nasal swab collection, collect the nasal | | | |
| | | before the pharyngeal swab. | | | |
| | Facility will aliquot 1 mL DNA Shield into an appropriate sized container. Following | | | | |
| | collection in the field the samples will be maintained at room temperature and | | | | |
| | transported to the lab where the samples will be stored at −80° C until shipped as a | | | | |
| | single batch at room temperature or on dry ice. | | | | |
| Nasal | 1-4 | C = collected using hydraflock swab placed | 1 per | 0, 2, 5, 10 | C = IBR & |
| Swab B | | in 2 mL PBS. Note: On SDs with both | calf | | BVDV qPCR |
| | | pharyngeal and nasal swab collection, | (C) | | |
| | | collect the nasal before the pharyngeal | | | |
| | | swab. | | | |
| | Facility will aliquot the PBS into appropriate containers. Following collection in the | | | | |
| | field samples will be transported to the lab on ice/icepacks. At the lab the tubes with | | | | |
| | the nasal swabs and PBS will be briefly vortexed (a few seconds) and then have the | | | | |
| | swab removed and discarded. The PBS will then be equally divided into 2 aliquots. | | | | |
| | Aliquots will be stored at ≤−60° C. until 1 aliquot is transported to ISUVDL on dry ice at | | | | |
| | study completion and the second is held in retention until analysis is complete at | | | | |
| | ISUVDL. | | | | |
| Nasal | 1 & 2 | D = collected using hydraflock swab and placed into a 2 mL Eppendorf tube | 1 per | 0, 2, 5, 10 | D = meta- |
| Swab C | (only 4 | containing 1 mL of RNA Later. Note: On | calf | | Genomics |
| | calves/TG | SDs with both pharyngeal and nasal swab | (D) | | |
| | | collection, collect the nasal before the | | | |
| | | pharyngeal swab | | | |
| | Greenfield will provide 2 mL Eppendorf tubes containing 1 mL of RNA Later. Samples | | | | |
| | will be stored at −80° C and shipped to Greenfield as a single batch on dry ice. | | | | |
| Serum D | All | D = 1 × 10 mL SST | 1 per | 0 & 24 | F = IBR & |
| | | | calf | | BVDV |
| | | | (F) | | serology |
| | Following collection into SSTs the blood tubes will be held at room temperature. The | | | | |
| | SSTs will be allowed to clot, will then be centrifuged and sera will be removed and | | | | |
| | divided into 2 equal aliquots (1 aliquot for IBR and BVDV serology and 1 aliquot for | | | | |
| | retention). Aliquots will be stored at ≤ -60° C. until shipped to ISUVDL on dry ice. One | | | | |
| | submission will occur at study conclusion. | | | | |
| Tracheal | 1 & 2 | G = During necropsy place 1 Carmalt | 1 per | 24 | G = meta- |
| Lavage | (only 4 | hemostat across the trachea just distal to | calf | | genomics |
| E | calves/TG | the larynx and 1 Carmalt hemostat across | (G) | | |
| | | the trachea between the junction of the | | | |
| | | proximal ¾ and distal ¼ of the trachea. | | | |
| | | Following clamping with the Carmalt | | | |
| | | hemostats transect the trachea free from | | | |
| | | the pluck. Remove the distal Carmalt and | | | |
| | | add 50 mL of normal saline to the lumen | | | |
| | | of the trachea. Replace the Carmalt and | | | |
| | | invert the trachea several times followed | | | |
| | | by removing the distal Carmalt and draining | | | |
| | | the normal saline into a sterile | | | |
| | | container. Using a sterile needle and | | | |
| | | syringe remove 1.5 mL of the saline lavage | | | |
| | | fluid and place in a 2 mL Eppendorf tube. | | | |
| | Facility will supply the normal saline lavage fluid as well as the 2 mL Eppendorf tubes. | | | | |
| | Following collection in the field the samples will be maintained at room temperature | | | | |
| | and stored at −80° C at Fort Dodge shipped as a single batch at study completion on dry ice. | | | | |
| Lung | 1 & 2 | I = During necropsy place 1 Carmalt | 1 per | 24 | I = meta- |
| Lavage F | (only 4 | hemostat across the trachea 10-12 inches | calf | | genomics |
| | calves/TG | proximal to the bifurcation prior to | (I) | | |
| | | removing the pluck from the calf. Once | | | |
| | | the pluck is removed from the calf, | | | |
| | | remove the Carmalt hemostat and using | | | |
| | | sterile technique use a hand pipettor to | | | |
| | | place 75 ml of normal saline into the | | | |
| | | trachea and then massage/distribute the | | | |
| | | lavage fluid. Using sterile technique and | | | |
| | | the hand pipettor remove 1.5 mL of the | | | |
| | | saline lavage fluid and place in a 2 mL | | | |
| | | Eppendorf tube. | | | |
| | Facility will supply the normal saline lavage fluid as well as the 2 mL Eppendorf tubes. | | | | |
| | Following collection in the field the samples will be maintained at room temperature | | | | |
| | and stored at −80° C at Fort Dodge shipped as a single batch at study completion on | | | | |
| | dry ice. | | | | |
| Lung | 1 & 2 | K = collect 1-2 grams of lung tissue that is | 1 per | 24 | K = meta- |
| | (only 4 | diseased and place in 1 ml of RNA Later | | | |
| Tissue G | calves/TG | contained in Eppendorf tube. Please | calf | | genomics |
| | | specify lung lobe sample is collected from | (K) | | |
| | | using the lung lobe identifier on the | | | |
| | | scoring form. | | | |
| | Facility will provide 2 mL Eppendorf tubes containing 1 ml of RNA Later. Samples will | | | | |
| | be stored at −80° C and shipped as a single batch at study conclusion on dry ice. | | | | |
| Lung | 1 & 2 | N = Each 1 × 1 cm piece of lung tissue (1 | 2 per | 24 | N = tissue |
| Tissue H | (only 4 | piece of diseased and 1 piece of healthy) | calf | | immune |
| | calves/TG | will be placed individually into a sterile | (N) | | parameters |
| | | Falcon/similar type tube (approximately | | | by qPCR |
| | | 15 mL) with a sufficient volume (5-10 | | | |
| | | volumes worth) of RNAlater ® to | | | |
| | | completely cover the samples. | | | |
| | Facility will prepare and tubes can be prefilled with RNAlater ® or added to the tubes | | | | |
| | when returned from the field. To prepare samples for storage at −20° C., first incubate | | | | |
| | the samples in RNAlater ® solution overnight at 4° C. to allow thorough penetration of | | | | |
| | the tissue, then transfered to −20° C. Ship on dry ice. | | | | |
| Lung | All | P = Each 1 × 1 cm piece of lung tissue (1 | 2 per | 24 | O = tissue |
| Tissue I | | piece of diseased and 1 piece of healthy) | calf | | gene |
| | | will be placed together into a sterile | (O) | | sequencing |
| | | Falcon/similar type tube (approximately | | | |
| | | 15 mL) with a sufficient volume of | | | |
| | | RNAlaterformalin. After formalin fixation | | | |
| | | for 2 days the samples are then moved to | | | |
| | | 90-100% ethanol. | | | |

| | Tubes can be prefilled with RNAlater ® or added to the tubes when returned from the | | | |
|---|---|---|---|---|
| | field. To prepare samples for storage at −20° C., first incubate the samples in RNAlater ® | | | |
| | solution overnight at 4° C. to allow thorough penetration of the tissue, | | | |
| | then transfer to −20° C. Ship on dry ice. | | | |
| Microbiome | 1 & 2 | All samples will be placed into the respective sample | 24 | P = |
| J | (only 4 | Falcon tube, which contains media consisting of BHI | | microbiome |
| | calves/TG | with 0.5% L-cysteine and 20% glycerol. Samples need | | |
| | | to be stored at 4° C. (on ice) for 2 hours after | | |
| | | collection and then stored at ≤−60° C. until shipment | | |
| | | on dry ice as a single submission. Tubes with media | | |
| | | will be provided from facility. | | |

### Necropsy

All Contact calves surviving to study conclusion will be euthanized (sedated with xylazine, rendered unconscious via captive bolt, and exsanguinated/pithed or administered pentobarbital intravascularly). Following euthanasia each calf will be necropsied, lungs scored, and samples collected according to Table 23. All Seeder calves surviving to SD 11 will be euthanized with lungs scored and no postmortem samples will be collected.

Any calf that dies/euthanized prior to the scheduled necropsy (SD 11 for Seeders and SD 24 for Contacts) will have all final SD activities completed. Weights used to calculate anesthesia and euthanasia solution, as applicable, may be by visual observation or using a recent weight.

Clinical Symptoms Associated with BRD:

Mortality, morbidity, coughing, nasal discharge, dyspnea/tachypnea, depression, pneumonia, diarrhea, and keratoconjunctivitis occurring post-challenge are to be expected and will not be considered adverse events. However, these will be documented in the final study report (FSR).

### Animal Management and Housing

### Penning

This study will occur indoors in the LAC facility and penning will consist of portable style paneling arranged in configurations to meet animal welfare standards as well as the design of the study. Contact and Seeder calves will be penned separately without nose-to-nose contact during acclimation. On SD 0 the Seeder calves will be inoculated and will then be commingled with the Contact calves until removal of the Seeder calves on SD 11.

### Bedding

Bedding will be straw or corn stalks or other similar bedding. From calf arrival until SD 0 bedding will be kept clean and dry. Beginning on SD 0 bedding will be allowed to become soiled and wet to mimic field conditions and other conditions as appropriate. Bedding will be replaced as needed as determined by a site veterinarian or trained designee. However, the soiled and wet bedding is an integral part of this model in mimicking field conditions and every effort will be made to mimic the an appropriate bedding regimen while meeting the animal welfare needs of the calves. On SD 13 normal/routine bedding practices will be restored as well as ventilation in the LAC.

### Feed and Water

Calves will have ad libitum access to fresh water and will be fed a grain ration once daily for the entirety of the study.

### Ventilation, Ambient Temperature, and Relative Humidity

From calf arrival until SD 0 the ventilation will be maintained at a minimum of 14 air exchanges/hour and a room temperature of approximately 60-70° F.. This will be adjusted during acclimation if determined that additional ventilation is required to maintain the health of the calves until SD 0.

On SD 0 the facility room temperature and relative humidity will be maintained at levels that provide for a Temperature Humidity index (THI) of 71 or less (see Table 24). Maintenance of an acceptable THI will be accomplished by modulating the air exchange settings of the facility air handler as well as twice daily observations for the room temperature and relative humidity. Environmental room conditions will be recorded using the automated facility room monitoring system throughout the study and will be summarized in the final report. On SD 13 the ventilation and temperature will be returned to standard/acclimation settings for the LAC.

The facility air handler will initially be set to minimize the number of air exchanges per hour. Therefore, adjustments may be made after animal arrival to the end of study as necessary to maintain an acceptable THI. Excursions where the THI is 72 or greater are permissible for up to 2 hours. Otherwise, excursions >2 hours will be documented with date, SD(s), maximum temperature, maximum relative humidity, and duration of the excursion in the study master file. Initially, the room temperature will be set at 68° F. with the plant monitoring system to alarm at ±4° F.. Temperature and humidity data during the study will be compiled from the automated plant monitoring system at the conclusion of the study.

TABLE 24

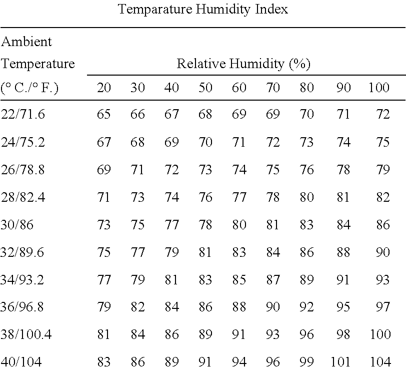

Note:
Shaded boxes represent temperature and humidity combinations that result in a THI of 72 or greater. These combinations should be avoided by adjusting the air-handling unit of the facility.

### Space Allocation Per Animal

Contact and Seeder calf space allocations will be at the minimum to meet the specifications cited in the Guide for the Care and Use of Agricultural Animals in Research and Teaching from arrival until SD 0.

However, following introduction of the Seeder calves (SD 0) into the Contact pen the floor space/area will be reduced by half for 6-8 hours/day, during normal facility hours, until the PM on SD 6. This floor space reduction will encourage nose-to-nose contact between the Seeder and Contact calves.

### Control Products (CP)

The negative CP is saline, TG 1 and will be dosed according to Table 17. Table 25 includes details for the positive control.

TABLE 25
Draxxin (Tulathromycin) Details

| | |
|---|---|
| Name of IVP: | Draxxin (Tulathromycin) |
| Formulation: | Tulathromycin injectable solution |
| TGs Administered: | 2 |
| SD Administered: | 0 |
| Serial/Lot | TBD. Will be included in the study file and FSR |
| Number/ID: | |
| Manufacturer: | Zoetis Inc. |
| Storage Conditions: | Store at or below 25° C. (77° F.). |
| Field Use: | According to the label |
| Transport | Transport at or below 25° C. (77° F.) |
| Conditions to Field | |
| for Use: | |
| Appearance: | Colorless to slightly yellow |
| Concentration: | 100 mg/ml |
| Dose: | 2.5 mg/kg |
| Route: | SC |
| Location: | Neck |

### Outcome Variables and Statistical Plan

### Primary Outcome Variable

Percent Lung Lesion Score at time of removal or study completion. Individual total lung lesion score will be calculated as follows:

Total Lung Lesion Score=(Right Cranial Score×0.06)+(Right Posterior Cranial Score×0.05)+(Right Middle Score×0.07)+(Right Caudal Score×0.35)+(Right Accessory Score×0.04)+(Left Cranial Score×0.05)+(Left Posterior Cranial Score×0.06)+(Left Caudal Score×0.32)

### Secondary Outcome Variables

1. Initial Onset of Clinical BRD during SD 0 to SD 24. Clinical BRD (treatment failure) is defined as: A rectal temperature of ≥104.0° F. in conjunction with a score of ≥2 for at least 1 of the 2 clinical observations (See Table 22) during the same observation period (AM or PM).
2. Mortality including the removal and euthanasia of moribund animals and animals found dead, from SD 0 to SD 24.
3. Gene sequencing on pharyngeal swabs.
4. IBR and BVDV qPCR on nasal swabs.
5. IBR and BVDV serology.
6. Cytokine analysis on tonsil and lung tissue.
7. Bacteriology on consolidated/pneumonic lung.
8. Lung and tonsil gene sequencing.

TABLE 26
Abbreviations and Definitions

| | |
|---|---|
| AE | Adverse Event |
| BRD | Bovine Respiratory Disease |
| BVD/BVDV | Bovine Viral Diarrhea Virus |
| BW | Body Weight |
| CD | Colostrum-Deprived |
| CFR | Code of Federal Regulation |
| CP | Control Product |
| CRF | Case Report Form |
| CRL | Charles River Laboratory |
| EDTA | Ethylenediaminetetraacetic Acid |
| EAH-US | Elanco Animal Health United States |
| FSR | Final Study Report |
| GPV | Global Pharmacovigilance |
| GXP | Good (X = clinical, laboratory, manufacturing, etc.) Practice |
| IACUC | Institute of Animal Care and Use Committee |
| IBR | Infectious Bovine Rhinotracheitis |
| ID | Identification |
| IN | Intranasal |
| ISUVDL | Iowa State University Veterinary Diagnostic Laboratory |
| IVP | Investigational Veterinary Product |
| LAC | Large Animal Challenge |
| Mh | Mannheimia haemolytica |
| MS | Microsoft |
| N | Number |
| NA | Not Applicable |
| PHMB | polyhexamethylene biguanide (also known as polyhexanide) |
| PI | Persistently Infected |
| pk-pd | Pharmacokinetic-Pharmacodynamic |
| PMN | Polymorphonuclear |
| SD | Study Day |
| SG | Seeder Group |
| SST | Serum Separator Tube |
| SQ | Subcutaneous |
| TBD | To Be Determined |
| TCID | Tissue Culture Infection Dose |
| TG | Treatment Group(s) |
| THI | Temperature Humidity Index |

The present disclosure may be embodied in other forms or carried out in other ways without departing from the spirit or essential characteristics thereof. The present disclosure is therefore to be considered as in all aspects illustrated and not restrictive, the scope of the disclosure being Indicated by the appended Claims, and all changes which come within the meaning and range of equivalency are Intended to be embraced therein.

Various references are cited throughout this Specification, each of which Is Incorporated herein by reference in its entirety.

1. An engineered antimicrobial polypeptide variant selected from:
a) a variant of CAP18, said polypeptide comprising:
(SEQ ID NO: 103)
X₁X₂X₃KX₄X₅X₆KX₇X₈NKIKEKLKKIGQKIQGLLPKLAPX₉TDX₁₀,
wherein X1 is G or CWTKSIPPKPC-G (SEQ ID NO: 104),
X2 is C or L,
X3 is R or K,
X4 is P, A, V, I, L, M, F, Y, or W,
X5 is C or L,
X6 is R or K,
X7 is I or K,
X8 is R, I, or K,
X9 is R or K, and
X10 is Y or Y-CWTKSIPPKPC (SEQ ID NO: 105),
wherein the polypeptide does not comprise GLRKRLRKFRNKIKEKLKKIGQKIQGLLPKLAPRTDY (SEQ ID NO: 1);
b) a variant of CAP18, said polypeptide comprising
(SEQ ID NO: 106)
GX₁RKX₂X₃X₄KX₅X₆NKIKEKLKKIGQKIQGLLPKLAPX₇TDY,
wherein X1 is C or L,
X2 is R, K, I, or P.
X3 is L or C,
X4 is L or R,
X5 is I,
X6 is R or K, and
X7 is R or K;
c) a variant of BMAP28, the polypeptide comprising:
(SEQ ID NO: 107)
X₁GX₂X₃SLGX₄KX₅LX₆AX₇KKX₈GPX₉IVPIIX₁₀IG,
wherein X1 is G, CWTKSIPPKPC-G (SEQ ID NO: 104) or CRKP-G (SEQ ID NO: 10⁸),
X2 is L or A,
X3 is R or K,
X4 is R or K,
X5 is I or A,
X6 is R or K,
X7 is W, I or A,
X8 is Y, I or A,
X9 is I or A, and
X10 is R or K,
wherein the polypeptide does not comprise GGLRSLGRKILRAWKKYGPIIVPIIRIG (SEQ ID NO: 50);
d) a truncated variant of BMAP28, the polypeptide comprising:
(SEQ ID NO: 109)
XGLRSLGRKILRAWKKYG,
wherein X is G, CWTKSIPPKPC-G (SEQ ID NO: 104), or CRKP-G (SEQ ID NO: 10⁸);
e) variant of BAC7, the polypeptide comprising:
(SEQ ID NO: 110)
X₁X₂IRPRPPX₃LPRPRPRPLPX₄PRPGPRPIPRPLPX₅PRPGPRPIPRPL
PX₆PRPGPRPIPRPL,
wherein X1 is R, CWTKSIPPKPC-R (SEQ ID NO: 111), CRKP-R (SEQ ID NO: 112) or K,
X2 is R or K
X3 is R or K,
X4 is F or I
X5 is F or I, and
X6 is F or I,
wherein the polypeptide does not comprise
(SEQ ID NO: 63)
RRIRPRPPRLPRPRPRPLPFPRPGPRPIPRPLPFPRPGPRPIPRPLPFP
RPGPRPIPRPL;
f) a variant of BAC7, the polypeptide comprising:
(SEQ ID NO: 110)
X₁X₂IRPRPPX₃LPRPRPRPLPX₄PRPGPRPIPRPLPX₅PRPGPRPIPRPL
PX₆PRPGPRPIPRPL,
wherein X1 is R, CWTKSIPPKPC-R (SEQ ID NO: 111), CRKP-R (SEQ ID NO: 112) or K,
X2 is R or K
X3 is R or K,
X4 is F or I
X5 is F or I, and
X6 is F or I,
wherein the polypeptide does not comprise
(SEQ ID NO: 63)
RRIRPRPPRLPRPRPRPLPFPRPGPRPIPRPLPFPRPGPRPIPRPLPFP
RPGPRPIPRPL;
g) a truncated variant of BAC7, the polypeptide comprising:
(SEQ ID NO: 113)
X₁X₂IRPRPPX₃LPRPRPR,
wherein X1 is R, CWTKSIPPKPC-R (SEQ ID NO: 111) or CRKP-R (SEQ ID NO: 112),
X2 is R or K, and
X3 is R or K;
h) a variant of K9CATH, the polypeptide comprising:
(SEQ ID NO: 114)
X₁LKELITTGGQKIGEKIX₂X₃IGQRIKDX₄X₅KNLQPX₆EEKS,
wherein X1 is R, CWTKSIPPKPC-R (SEQ ID NO: 111), CRKP-R (SEQ ID NO: 112) or K,
X2 is R or K,
X3 is R or K,
X4 is F or I,
X5 is F or I, and
X6 is R or K,
wherein the polypeptide does not comprise
(SEQ ID NO: 77)
RLKELITTGGQKIGEKIRRIGQRIKDFFKNLQPREEKS;
i) a truncated variant of K9CATH, the polypeptide comprising:
(SEQ ID NO: 115)
X₁LKELITTGGQKIGEKIX₂X₃IG,
wherein X1 is R, CWTKSIPPKPC-R (SEQ ID NO: 111) or CRKP-R (SEQ ID NO: 112),
X2 is R or K, and
X3 is R or K;
i) a truncated variant of PMAP36, the polypeptide comprising:
(SEQ ID NO: 116)
X₁X₂X₃RX₄LRKX₅TRX₆X₇LKX₈IGKVLKX₉I,
wherein X1 is G, CWTKSIPPKPC-G (SEQ ID NO:104) or CRKP-G (SEQ ID NO: 10⁸),
X2 is R or V,
X3 is F or L,
X4 is R or V,
X5 is K or V,
X6 is K or V,
X7 is R or V,
X8 is K or V, and
X9 is W or L or
k) a truncated 2 variant of PMAP36, the polypeptide comprising:
(SEQ ID NO: 117)
X₁X₂LRKKTRKRLKKIGKVLKX₃I,
wherein X1 is R or CWTKSIPPKPC-R (SEQ ID NO: 111),
X2 is R or K, and
X3 is W or L.

2. The engineered polypeptide variant of CAP18 of claim 1 subpart a) wherein the polypeptide comprises one of the following:
(SEQ ID NO: 13)
GLRKRLRKIRNKIKEKLKKIGQKIQGLLPKLAPRTDY;
(SEQ ID NO: 49)
GLRKRLRKIRNKIKEKLKKIGQKIQGLLPKLAPRTDYCWTKSIPPKPC;
(SEQ ID NO: 46)
CWTKSIPPKPCGLRKRLRKIRNKIKEKLKKIGQKIQGLLPKLAPRTDY;
(SEQ ID NO: 47)
CWTKSIPPKPCGLRKRLRKIRNKIKEKLKKIGQKIQGLLPKLAPRTDYC
WTKSIPPKPC;
(SEQ ID NO: 18)
CWTKSIPPKPCGLRKRLKKIKNKIKEKLKKIGQKIQGLLPKLAPRTDY;
(SEQ ID NO: 19)
CWTKSIPPKPCGLRKILKKIKNKIKEKLKKIGQKIQGLLPKLAPRTDY;
(SEQ ID NO: 20)
CWTKSIPPKPCGLRKKLKKIRNKIKEKLKKIGQKIQGLLPKLAPRTDY;
(SEQ ID NO: 21)
CWTKSIPPKPCGLRKRLRKIKNKIKEKLKKIGQKIQGLLPKLAPKTDY;
(SEQ ID NO: 22)
CWTKSIPPKPCGCRKPLRKIRNKIKEKLKKIGQKIQGLLPKLAPRTDY;
(SEQ ID NO: 23)
CWTKSIPPKPCGCRKPCRKIRNKIKEKLKKIGQKIQGLLPKLAPRTDY;
(SEQ ID NO: 30)
CWTKSIPPKPCGLRKRLRKIRNKIKEKLKKIGQKIQGLLPKLAPRTDY;
(SEQ ID NO: 31)
GLRKRLKKIKNKIKEKLKKIGQKIQGLLPKLAPRTDY;
(SEQ ID NO: 32)
GLRKILKKIKNKIKEKLKKIGQKIQGLLPKLAPRTDY;
(SEQ ID NO: 33)
GLRKKLKKIRNKIKEKLKKIGQKIQGLLPKLAPRTDY;
(SEQ ID NO: 34)
GLRKRLRKIKNKIKEKLKKIGQKIQGLLPKLAPKTDY;
(SEQ ID NO: 35)
GCRKPLRKIRNKIKEKLKKIGQKIQGLLPKLAPRTDY;
(SEQ ID NO: 36)
GCRKPCRKIRNKIKEKLKKIGQKIQGLLPKLAPRTDY;
(SEQ ID NO: 37)
GLRKKLKKIKNKIKEKLKKIGQKIQGLLPKLAPRTDY;
(SEQ ID NO: 38)
GLKKKLKKIKNKIKEKLKKIGQKIQGLLPKLAPRTDY;
(SEQ ID NO: 39)
GLKKKLKKIKNKIKEKLKKIGQKIQGLLPKLAPKTDY;
(SEQ ID NO: 40)
GLRKILRKIRNKIKEKLKKIGQKIQGLLPKLAPRTDY;
(SEQ ID NO: 41)
GLKKILKKIKNKIKEKLKKIGQKIQGLLPKLAPRTDY;
or
(SEQ ID NO: 42)
GLKKKLRKIRNKIKEKLKKIGQKIQGLLPKLAPRTDY.

3. (canceled)

4. The engineered polypeptide variant of CAP18 according to claim 1 subpart b), wherein the polypeptide comprises one of the following:
(SEQ ID NO: 30)
CWTKSIPPKPCGLRKRLRKIRNKIKEKLKKIGQKIQGLLPKLAPRTDY;
(SEQ ID NO: 31)
GLRKRLKKIKNKIKEKLKKIGQKIQGLLPKLAPRTDY;
(SEQ ID NO: 32)
GLRKILKKIKNKIKEKLKKIGQKIQGLLPKLAPRTDY;
(SEQ ID NO: 33)
GLRKKLKKIRNKIKEKLKKIGQKIQGLLPKLAPRTDY;
(SEQ ID NO: 34)
GLRKRLRKIKNKIKEKLKKIGQKIQGLLPKLAPKTDY;
(SEQ ID NO: 35)
GCRKPLRKIRNKIKEKLKKIGQKIQGLLPKLAPRTDY;
or
(SEQ ID NO: 36)
GCRKPCRKIRNKIKEKLKKIGQKIQGLLPKLAPRTDY.

5. (canceled)

6. The engineered polypeptide variant of BMAP28 of claim 1 subpart c) wherein the polypeptide comprises one of the following:
(SEQ ID NO: 51)
CWTKSIPPKPCGGLRSLGRKILRAWKKYGPIIVPIIRIG;
(SEQ ID NO: 53)
CRKPGGLRSLGRKILRAWKKYGPIIVPIIRIG;
(SEQ ID NO: 55)
GGLKSLGKKILRAWKKYGPIIVPIIRIG;
(SEQ ID NO: 56)
GGLRSLGKKILKAWKKYGPIIVPIIRIG;
(SEQ ID NO: 57)
GGLRSLGRKILKAWKKYGPIIVPIIKIG;
(SEQ ID NO: 58)
GGLKSLGKKILKAWKKYGPIIVPIIKIG;
(SEQ ID NO: 59)
GGLRSLGRKILRAIKKYGPIIVPIIRIG;
(SEQ ID NO: 60)
GGLRSLGRKILRAWKKIGPIIVPIIRIG;
(SEQ ID NO: 61)
GGLRSLGRKILRAIKKIGPIIVPIIRIG;
or
(SEQ ID NO: 62)
GGARSLGRKALRAAKKAGPAIVPIIRIG.

7. (canceled)

8. (canceled)

9. The engineered polypeptide variant of BAC7 of claim 1 subpart f) comprising:
(SEQ ID NO: 64)
CWTKSIPPKPCRRIRPRPPRLPRPRPRPLPFPRPGPRPIPRPLPFPRPG
PRPIPRPLPFPRPGPRPIPRPL;
(SEQ ID NO: 65)
CRKPRRIRPRPPRLPRPRPRPLPFPRPGPRPIPRPLPFPRPGPRPIPRP
LPFPRPGPRPIPRPL;
(SEQ ID NO: 70)
RRIRPRPPRLPRPRPRPLPIPRPGPRPIPRPLPIPRPGPRPIPRPLPFP
RPGPRPIPRPL;
(SEQ ID NO: 71)
RRIRPRPPRLPRPRPRPLPFPRPGPRPIPRPLPIPRPGPRPIPRPLPIP
RPGPRPIPRPL;
(SEQ ID NO: 72)
RRIRPRPPRLPRPRPRPLPIPRPGPRPIPRPLPIPRPGPRPIPRPLPIP
RPGPRPIPRPL;
(SEQ ID NO: 73)
KKIRPRPPRLPRPRPRPLPFPRPGPRPIPRPLPFPRPGPRPIPRPLPFP
RPGPRPIPRPL;
(SEQ ID NO: 74)
RKIRPRPPKLPRPRPRPLPFPRPGPRPIPRPLPFPRPGPRPIPRPLPFP
RPGPRPIPRPL;
or
(SEQ ID NO: 75)
KKIRPRPPKLPRPRPRPLPFPRPGPRPIPRPLPFPRPGPRPIPRPLPFP
RPGPRPIPRPL.

10. (canceled)

11. The engineered polypeptide truncated variant of BAC7 of claim 1 subpart g) comprising one of the following:
(SEQ ID N.: 66)
RRIRPRPPRLPRPRPR;
(SEQ ID NO: 67)
CWTKSIPPKPCRRIRPRPPRLPRPRPR;
(SEQ ID NO: 68)
CRKPRRIRPRPPRLPRPRPR;
or
(SEQ ID NO: 76)
KKIRPRPPKLPRPRPR.

12. (canceled)

13. The engineered polypeptide variant of K9CATH of claim 1 subpart h) comprising:
(SEQ ID NO: 83)
RLKELITTGGQKIGEKIRRIGQRIKDIIKNLQPREEKS;
(SEQ ID NO: 84)
KLKELITTGGQKIGEKIKRIGQRIKDFFKNLQPREEKS;
(SEQ ID NO: 85)
RLKELITTGGQKIGEKIKKIGQRIKDFFKNLQPREEKS;
(SEQ ID NO: 86)
RLKELITTGGQKIGEKIRKIGQRIKDFFKNLQPKEEKS;
or
(SEQ ID NO: 87)
RLKELITTGGQKIGEKIKKIGQRIKDFFKNLQPKEEKS.

14. (canceled)

15. The engineered polypeptide truncated variant of K9CATH of claim 1 subpart i) comprising:
(SEQ ID NO: 88)
RLKELITTGGQKIGEKIKKIG;
or
(SEQ ID NO: 89)
CWTKSIPPKPCRLKELITTGGQKIGEKIKKIG.

16. (canceled)

17. The engineered polypeptide truncated variant of PMAP36 of claim 1 subpart i) comprising one of the following:
(SEQ ID NO: 96)
GRFRRLRKKTRKRLKKIGKVLKLI;
(SEQ ID NO: 97)
GRLRRLRKKTRKRLKKIGKVLKLI;
(SEQ ID NO: 98)
GVFRVLRKVTRVVLKVIGKVLKLI;
or
(SEQ ID NO: 99)
GVLRVLRKVTRVVLKVIGKVLKLI.

18. (canceled)

19. The engineered polypeptide truncated 2 variant of PMAP36 of claim 1 subpart k) comprising:
(SEQ ID NO: 102)
CWTKSIPPKPCRKLRKKTRKRLKKIGKVLKLI.

20. An antimicrobial composition comprising:
one or more said engineered polypeptide according to claim 1; and
a pharmaceutically acceptable carrier.

21. A method of treating a microbial or parasitic infection or for reducing greenhouse gas emissions, said method comprising:
administering to a subject or an animal in need thereof, the antimicrobial composition of claim 20.

22. The method according to claim 21, wherein the microbial infection is caused by Mannheimia haemolytica, Pasteurella multocida, E. coli, Salmonella, C. jejuni, P. salmonis, Giardia or Eimeria.

23. (canceled)

24. (canceled)

25. A method of claim 21 wherein the engineered polypeptide is administered to an animal in an amount effective to inhibit growth of at least one methanogen in the animal.

26. The method of claim 25 wherein said at least one methanogen comprises at least one of Methanobrevibacter ruminantium DSM 1093, Methanosphaera stadtmanae DSM 3091, Methanomicrobium mobile DSM 1539, Methanobacterium bryantii, Methanobrevibacter gottchackii, Methanobrevibacter olleyae, Methanobrevibacter thauerii, Methanomassilicoccus luminyensis or Methanosarcina barkeri, and combinations thereof.

27. The method of claim 25 wherein said engineered polypeptide targets cell membranes of said at least one methanogen, said at least one methanogen being located in the gastrointestinal tract of an animal.

28. The method of claim 214 wherein the animal comprises one or more of: cattle which include cows, bulls and calves; poultry which includes broilers, chickens and turkeys; pigs which include piglets; birds; aquatic animals which include fish, agastric fish, gastric fish, freshwater fish which include salmon, cod, trout and carp, marine fish which include salmon and sea bass, and crustaceans which include shrimps, mussels and scallops; horses which include race horses; and sheep which include lambs.

29. The method of claim 25 wherein the animal is a ruminant comprising at least one of extensive beef cattle, intensive beef cattle and dairy cattle.

30. The method of claim 29 wherein said administration is effective in reducing enteric methane gas emissions from said ruminant in an amount of at least 20%, 30%, 40%, 50% or 60%.

31. The method of claim 21 wherein said engineered polypeptide is conjugated or attached to one or more other molecules or agents comprising at least one of peptides conjugated to a cell or pathogen targeting agent or sequence, toxin, immunomodulator, cytokine, cytotoxic agent, or one or more anti-bacterial, anti-parasitic or anti-viral agent or drug.

32. (canceled)

33. The method of claim 21 comprising administering said engineered polypeptide in combination with or coadministering with one or more prebiotic or with another agent comprising at least one of an anti-bacterial agent, anti-infective agent, or an immunomodulatory agent, and combinations thereof.

34. The method of claim 21 wherein said engineered polypeptide is administered as part of a composition comprising at least one of animal feed, a feed additive, a food ingredient, a water additive, a water-mixed additive, a consumable solution, a consumable spray additive, a consumable solid, a consumable gel, an injectable, or combinations thereof and wherein administration is carried out orally or by injection.

35. (canceled)

36. The method of claim 21 wherein said engineered polypeptide is administered as part of a pharmaceutical composition for oral administration in a tablet, a capsule, a powder or a liquid form, said pharmaceutical composition comprising a pharmaceutically acceptable carrier.

37. The method of claim 21 wherein said engineered polypeptide is administered as part of a composition including one or more biologically active molecule or therapeutic molecule comprising at least one of an ionophore; a vaccine; an antibiotic; an antihelmintic; a virucide; a nematicide; amino acids including methionine, glycine, or arginine; fish oil; krill oil; and enzymes.

38. A method comprising administering to an animal a unicellular host capable of heterologously expressing at least one of said engineered polypeptides of claim 1.

39. The method of claim 38 wherein said unicellular host is transformed by a vector that comprises nucleic acid encoding said engineered polypeptide, or wherein said unicellular host includes a genome into which heterologous nucleic acid encoding said engineered polypeptide has been integrated.

40. (canceled)

41. (canceled)

42. The method of claim 38 wherein said unicellular host is administered to the animal by intranasal spray, by injection, as part of a direct fed microbial, or by oral administration.
